(19) United States
(12) Patent Application Publication
(10) Pub. No.: US 2024/0050529 A1
(43) Pub. Date: Feb. 15, 2024

(54) MODULATING LYMPHATIC VESSELS IN NEUROLOGICAL DISEASE

(71) Applicant: University of Virginia Patent Foundation, Charlottesville, VA (US)

(72) Inventors: Jonathan Kipnis, Charlottesville, VA (US); Antoine Louveau, Charlottesville, VA (US); Sandro Da Mesquita, Charlottesville, VA (US)

(21) Appl. No.: 17/766,479

(22) PCT Filed: Oct. 6, 2020
(86) PCT No.: PCT/US2020/054390

§ 371 Date: Apr. 4, 2022

Related U.S. Application Data
(60) Provisional application No. 62/965,769, filed on Jan. 24, 2020.
(60) Provisional application No. 62/947,869, filed on Dec. 13, 2019.
(60) Provisional application No. 62/911,893, filed on Oct. 7, 2019.

(51) Int. Cl. A61K 38/18; A61P 17/18; A61K 45/06

(52) U.S. Cl. CPC A61K 38/1825; A61K 38/1866; A61P 17/18; A61K 45/06

(57) ABSTRACT

In some embodiments herein, methods, compositions, and uses for modulating lymphatic vessels of the central nervous system are described. In some embodiments, methods, compositions, or uses for treating, preventing, or ameliorating symptoms of a neurological disease comprise increasing flow via meningeal lymphatic vessels are described.

### RELATED APPLICATIONS

This application is related to and claims priority of U.S. Provisional Application No. 62/911,893, filed on Oct. 7, 2019, U.S. Provisional Application No. 62/947,869, filed on Dec. 13, 2019, and U.S. Provisional Application No. 62/965,769, filed on Jan. 24, 2020. The entire contents of each of the foregoing applications are expressly incorporated herein by reference.

### STATEMENT REGARDING FEDERALLY SPONSORED R&D

This invention was made with government support under Grant Nos. AG034113, AG057496 and NS061973 awarded by the National Institutes of Health. The government has certain rights in the invention.

### REFERENCE TO SEQUENCE LISTING

The present application is being filed along with a Sequence Listing in electronic format. The Sequence Listing is provided as a file entitled 131819-01120_SL.txt, created and last modified Oct. 6, 2020, which is 19,864 bytes in size. The information in the electronic format of the Sequence Listing is incorporated herein by reference in its entirety.

### BACKGROUND

Neurological diseases impact millions of people worldwide, and include degenerative and inflammatory neurological diseases. Among degenerative neurological diseases, Alzheimer's Disease (AD) is the most prevalent form of dementia worldwide (Andrieu et al., 2015) and is distinctively characterized by early and marked cognitive impairment (Andrieu et al., 2015; Ballard et al., 2011). The vast majority (>98%) of AD cases are sporadic (Blennow et al., 2006), and in such cases the etiology of the amyloid pathology is poorly understood (Benilova et al., 2012; Blennow et al., 2006). This is in contrast to familial AD, where rare hereditary dominant mutations in amyloid precursor protein (APP) or in presenilins 1 and 2 drive the uncontrolled formation of amyloid beta (Hardy and Selkoe, 2002). The brain's pathological hallmarks of AD are intracellular neurofibrillary tangles and extracellular amyloid plaques, the latter being a product of the amyloidogenic processing of APP and the resulting deposition of amyloid beta in the brain parenchyma (Benilova et al., 2012; Hardy and Selkoe, 2002; Ittner and Gotz, 2011). Increasing aggregation of diffusible amyloid beta peptides from the ISF and the CSF into toxic oligomeric intermediates and their accumulation in the brain parenchyma (Hong et al., 2011; Iliff et al., 2012) are believed to be precipitating factors for different neuroinflammatory abnormalities (Guillot-Sestier et al., 2015; Hong et al., 2016; Matarin et al., 2015), such as the formation of neurofibrillary tangles (Ittner and Gotz, 2011) and the pronounced neuronal dysfunction (Palop et al., 2007; Sun et al., 2009; Walsh et al., 2002) in the AD brain.

Organs generally function less effectively with age. For example, skin becomes less elastic, muscle tone is lost, and heart function declines. Aging is a substantial risk factor for numerous neurological diseases, including neurodegenerative diseases and inflammatory neurological diseases.

### FIELD

Several embodiments herein relate generally to compositions, methods, and uses for modulating lymphatic vessels in the central nervous system. Modulating lymphatic vessels, in accordance with some embodiments, are used to treat, prevent, or ameliorate symptoms of neurological diseases.

### SUMMARY

In one aspect, the present invention provides a method of increasing clearance of a molecule from the central nervous system in a subject in need thereof. The method includes administering an effective amount of a flow modulator to a meningeal space of the subject, wherein the flow modulator increases the fluid flow in the central nervous system of the subject; and administering an effective amount of a neurological therapeutic agent to the central nervous system of the subject, thereby increasing the clearance of the molecule from the central nervous system of the subject. In one embodiment, the clearance of a molecule from the central nervous system in a subject is increased by at least about 5%, about 10%, about 15%, about 20%, about 50%, about 100%, about 2 fold, or more. In another embodiment, the clearance of a molecule is measured by the accumulation of the molecule drained into the deep cervical lymph nodes, or the reduction of the molecule in the central nervous system, or both. In still another embodiment, the clearance of the molecule is the clearance from the brain of the subject.

In another aspect, the present invention provides a method of treating a neurological disease in a subject in need thereof. The method includes administering an effective amount of a flow modulator to a meningeal space of the subject, wherein the flow modulator increases the fluid flow in the central nervous system of the subject; and administering an effective amount of a neurological therapeutic agent for the neurological disease to the subject, thereby treating the neurological disease in the subject.

In still another aspect, the present invention provides a method of reducing an aggregate of a protein or peptide in a subject in need thereof. The method includes administering a flow modulator to a meningeal space of the subject, wherein the flow modulator increases the fluid flow in the central nervous system of the subject; and administering an effective amount of a neurological therapeutic agent to the subject, thereby reducing the aggregate of the protein or peptide in the subject. In one embodiment, the aggregate of the protein or peptide is reduced by at least about 5%, about 10%, about 15%, about 20%, about 50%, about 90%, or about 99%. In one embodiment, the reduction in the aggregate of the protein or peptide is measured by the reduction in the number of the aggregates, the average size of the aggregates, the coverage of the aggregate in a tissue section as the percentage of the area of the aggregate to the area of a tissue section, or any combination thereof. In another embodiment, the coverage of the aggregates is the percentage of the area covered by the aggregates to the area of a brain cross section. In still another embodiment, the reduction in the aggregates of the protein or peptide is observed and measured by in vivo magnetic resonance imaging. In yet another embodiment, the method reduces the protein or peptide aggregate in the brain of the subject.

In yet another aspect, the present invention provides a method of reducing a microglial inflammatory response in the central nervous system of a subject in need thereof. The method includes administering an effective amount of a flow modulator to a meningeal space of the subject, wherein the flow modulator increases the fluid flow in the central nervous system of the subject; and administering an effective amount of a neurological therapeutic agent to the subject, thereby reducing the microglial inflammatory response in the central nervous system of the subject. In one embodiment, the reduction of the microglial inflammatory response includes a reduction of the number of microglia surrounding a protein aggregate, reduction of the percentage of activated microglia to the total number of microglia, or both. In another embodiment, the methods reduces the microglial inflammatory response by at least about 5%, about 10%, about 15%, about 20%, about 50%, about 99%, or more. In still another embodiment, the methods reduce the microglia inflammatory response as measured by the reduction of the quantity of microglia surrounding a protein aggregate, reduction of the percentage of activated microglia to total microglia, or both. In one embodiment, the quantity of microglia is measured by measuring the microglial cell marker IBA. In another embodiment, the level of activated microglia is measured by measuring the activated microglia marker CD68. In still another embodiment, the method reduces the microglial inflammatory response in the brain of the subject.

In still another aspect, the present invention provides a method of reducing a neurite dystrophy in the central nervous system of a subject in need thereof. The method includes administering an effective amount of a flow modulator to a meningeal space of the subject, wherein the flow modulator increases the fluid flow in the central nervous system of the subject; and administering an effective amount of a neurological therapeutic agent for the neurological disease to the subject, thereby reducing the neurite dystrophy treating the neurological disease in the central nervous system of the subject. In one embodiment, the method reduces neurite dystrophy by at least about 5%, about 10%, about 15%, about 20%, about 50%, about 90%, or about 99%. In another embodiment, the neurite dystrophy is measured by the levels of lysosomal-associated membrane protein 1. In still another embodiment, the method reduces the neurite dystrophy in the brain of the subject.

In one embodiment of various aspects of the invention, the flow modulator comprises a lymphangiogenic growth factor or a polynucleotide comprising a sequence encoding the lymphangiogenic growth factor. In another embodiment, the lymphangiogenic growth factor comprises a VEGFR3 agonist or a FGF2, or a variant thereof. In still another embodiment, the VEGFR3 agonist comprises a VEGF-c, a VEGF-d, or a variant thereof. In yet another embodiment, the VEGFR3 agonist comprises a VEGF-c, or a variant thereof. In one embodiment, the VEGF-c variant comprises a VEGFC156S, or a VEGF-165.

In another embodiment, the method of the present invention increases the clearance of a molecule or reduces an aggregate of a protein or peptide, wherein the molecule or the protein or the peptide is selected from the group consisting of Aβ (amyloid beta), alpha synuclein, fibrin, tau, apolipoprotein E (Apoe), TDP43, prion protein, huntingtin, Huntingtin exon 1, ABri peptide, ADan peptide, fragments of immunoglobulin light chains, fragments of immunoglobulin heavy chains, full or N-terminal fragments of serum amyloid A protein (SAA), transthyretin (TTR), β₂-microglobulin, N-terminal fragments of apolipoproteinA-I (ApoAI), C-terminal extended apolipoprotein A-II (ApoAll), N-terminal fragments of apolipoprotein A-IV (ApoAIV), apolipoprotein C-II (ApoCII), apolipoprotein C-III (ApoAIII), fragments of gelsolin, lysozyme, fragments of fibrinogen α-chain, N-terminal truncated cystatin C, islet amyloid polypeptide (IAPP), calcitonin, atrial natriuretic factor (ANF), N-terminal fragments of prolactin (PRL), insulin, medin, lactotransferrin, odontogenic ameloblast-associated protein (ODAM), pulmonary surfactant-associated protein C (SP-C), leukocyte cell-derived chemotaxin-2 (LECT-2), galectin 7 (Gal-7), Corneodesmosin (CDSN), C-terminal fragments of kerato-epthelin (Pih-h3), semenogelin-1 (SGI), proteins S100A8/A9, Enfuvirtide, GSK-33, MARK, CDK5, tyrosine kinase Fyn, protein phosphatase 2A (PP2A), LRRK2, GBA, NF-κB p65. In one embodiment, the molecule or the protein or the peptide is selected from the group consisting of amyloid beta, fibrin, tau, apolipoprotein E (Apoe), alpha synuclein, TDP43, and huntingtin. In some embodiment, the aggregate of the protein or the peptide is located in the meninges of the subject's brain.

In still another embodiment, the neurological therapeutic agent comprises an agent selected from the group consisting of a small molecule, a nucleic acid, a peptide, a protein, an antibody or antigen binding fragment thereof, a recombinant virus, a vaccine, a cell, and any combination thereof.

In one embodiment, the neurological therapeutic agent comprises a small molecule agent. In another embodiment, the small molecule agent comprises any one or more agents in Table 2. In still another embodiment, the small molecule agent is selected from the group consisting of Donepezil, Galantamine, Rivastigmine, Memantine, Lanabecestat, Atabecestat, Verubecestat, Elenbecestat, Semagacestat, Tarenflurbil, and Brexipiprazole.

In another embodiment, the neurological therapeutic agent comprises an antibody, or an antigen binding fragment thereof. In one embodiment, the method increases the clearance of a molecule or reduces an aggregate of a protein or peptide, and the antibody or the antigen binding fragment thereof binds to the molecule or the protein or the peptide. In another embodiment, the molecule or the protein or the peptide is selected from the group consisting of amyloid precursor protein, amyloid beta, fibrin, tau, apolipoprotein E (Apoe), alpha-synuclein, TDP43, and huntingtin. In still another embodiment, the protein is amyloid precursor protein or amyloid beta, and the antibody or the antigen binding fragment thereof is selected from the group consisting of: bapineuzumab, gantenerumab, aducanumab, solanezumab, immunoglobulin, BAN2401, semorinemab, zagotenemab, crenezumab, and the antigen binding fragment thereof. In yet another embodiment, the protein is tau, and the antibody or the antigen binding fragment thereof is selected from the group consisting of Gosuranemab, Armanezumab, ABBV-8E12 (AbbVie), PHF1, MC1, DA31, 4E6G7, 6B2G12, TOMA, PHF6, PHF13, HJ9.3, HJ9.4, HJ8.5, 43D, 77E9, AT8, MAb86, pS404 mAb IgG2, pS409-tau, PHF1, Ta9, Ta4, Ta1505, DC8E8, and the antigen binding fragment thereof. In one embodiment, the protein is alpha-synuclein, and the antibody or the antigen binding fragment thereof is selected from the group consisting of BIIB054 (Biogen), PRX002/RG7935 (Roche), prasinezumab (Roche), PD-1601 (AbbVie), 1H7, 5C1, A1-A6, 9E4, 274, NbSyn87*PEST, NAC32, NAC1, AC14, VH14*PEST, syn303, AB1, Human single-chain Fv D10, D5, syn-O1, syn-O2, syn-O4, mAb47, syn-10H, syn-F1, syn-F2, LS4-2G12, and the antigen binding fragment thereof. In another embodiment, the antibody or the antigen binding fragment thereof is selected from the group consisting of bapineuzumab, gantenerumab, aducanumab, solanezumab, crenezumab, pepinemab, ozanezumab, AT-1501, BIIB054, PRX002, and the antigen binding fragment thereof.

In one embodiment, the flow modulator or the neurological therapeutic agent comprises a polynucleotide comprising a sequence encoding a protein or peptide flow modulator or neurological therapeutic agent. In another embodiment, the polynucleotide is comprised in an expression vector. In still another embodiment, the expression vector is a viral vector. In yet another embodiment, the viral vector is a adeno-associated virus vector.

In another embodiment, the flow modulator comprises a protein or a peptide and the neurological therapeutic agent comprises a protein or a peptide. In still another embodiment, the flow modulator comprises a protein or a peptide and the neurological therapeutic agent comprises a polynucleotide comprising a sequence encoding a protein or a peptide. In yet another embodiment, the flow modulator comprises a polynucleotide comprising a sequence encoding a protein or a peptide and the neurological therapeutic agent comprises a protein or a peptide. In one embodiment, the flow modulator comprises a polynucleotide comprising a sequence encoding a protein or a peptide and the neurological therapeutic agent comprises a sequence encoding a protein or a peptide.

In one embodiment, the flow modulator is different than the neurological therapeutic agent.

In still another embodiment, the flow modulator increases the diameter of the meningeal lymphatic vessel by at least about 5%, about 10%, about 15%, or about 20%. In one embodiment, the increase in the diameter of the meningeal lymphatic vessel is detected or measured by magnetic resonance imaging.

In one embodiment, the method of the present invention treats a neurological disease. The neurological disease is selected from the group consisting of: Alzheimer's disease, Parkinson's disease, cerebral edema, amyotrophic lateral sclerosis (ALS), Pediatric Autoimmune Neuropsychiatric Disorders Associated with Streptococcal Infections (PANDAS), meningitis, hemorrhagic stroke, Autism spectrum disorder (ASD), brain tumor, epilepsy, Down's syndrome, hereditary cerebral hemorrhage with amyloidosis-Dutch type (HCHWA-D), Familial Danish/British dementia, dementia with Lewy bodies (DLB), Lewy body (LB) variant of AD, multiple system atrophy (MSA), familial encephalopathy with neuroserpin inclusion bodies (FENIB), frontotemporal dementia (FTD), Huntington's disease (HD), Kennedy disease/spinobulbar muscular atrophy (SBMA), dentatorubropallidoluysian atrophy (DRPLA); spinocerebellar ataxia (SCA) type I, SCA2, SCA3 (Machado-Joseph disease), SCA6, SCA7, SCA17, Creutzfeldt-Jakob disease (CJD), familial CJD, Kuru, Gerstmann-Straussler-Scheinker syndrome (GSS), fatal familial insomnia (FFI), corticobasal degeneration (CBD), progressive supranuclear palsy (PSP), cerebral amyloid angiopathy (CAA), multiple sclerosis (MS), AIDS-related dementia complex, brain injury, traumatic brain injury, brain hemorrhage, subarachnoid brain hemorrhage, stroke, and any combination thereof.

In another embodiment, wherein the flow modulator is administered to the subject by a route selected from the group consisting of: intrathecal administration, intraventricular administration, intraparenchymal administration, nasal administration, transcranial administration, contact with cerebral spinal fluid (CSF) of the subject, pumping into CSF of the subject, implantation into the skull or brain, contacting a thinned skull or skull portion of the subject with the flow modulator, expression in the subject of a nucleic acid encoding the flow modulator, and any combination thereof.

In still another embodiment, the neurological therapeutic agent is administered to the subject by a route selected from the group consisting of: intrathecal administration, intraventricular administration, intraparenchymal administration, nasal administration, transcranial administration, contact with cerebral spinal fluid (CSF) of the subject, pumping into CSF of the subject, implantation into the skull or brain, contacting a thinned skull or skull portion of the subject with the neurological therapeutic agent, expression in the subject of a nucleic acid encoding the neurological therapeutic agent, intravenous infusion, and any combination thereof.

In one embodiment, the flow modulator and the neurologic therapeutic agent are formulated in a same pharmaceutical composition. In another embodiment, the flow modulator and the neurologic therapeutic agent are formulated in different pharmaceutical compositions.

In another embodiment, the flow modulator is administered to the subject prior to, concurrently with, or subsequent to the administration of the neurologic therapeutic agent.

In still another embodiment, the flow modulator is administered via a same route as the neurological therapeutic agent. In yet another embodiment, the flow modulator is administered via a different route to the neurological therapeutic agent.

In one embodiment, the neurological therapeutic agent is administered to the central nervous system (CNS) of the subject. In another embodiment, the neurological therapeutic agent is administered to the meninges of the subject's brain.

In one embodiment of various aspects of the invention, the fluid flow comprises the flow of cerebral spinal fluid (CSF), interstitial fluid (ISF), or both.

In one aspect, the present invention provides a method of increasing clearance of an amyloid beta, or an amyloid precursor protein, from the central nervous system in a subject in need thereof. The method includes administering an effective amount of a VEGF-c, or a variant thereof, or a polynucleotide encoding the VEGF, or the variant thereof, to a meningeal space of the subject, wherein the VEGF-c, or the variant thereof, increases the fluid flow in the central nervous system of the subject; and administering an effective amount of neurological therapeutic agent to the central nervous system of the subject, thereby increasing the clearance of the amyloid beta or the amyloid precursor protein from the central nervous system of the subject. In one embodiment, the clearance of the amyloid beta or the amyloid precursor protein from the central nervous system in a subject is increased by at least about 5%, about 10%, about 15%, about 20%, about 50%, about 100%, about 2 fold, or more. In another embodiment, the clearance of a molecule is measured by the amount of the molecule drained into the deep cervical lymph nodes. In still another embodiment, the method increases the clearance of the amyloid beta, or the amyloid precursor protein from the brain of the subject.

In another aspect, the present invention provides a method of treating an Alzheimer's disease in a subject in need thereof. The method includes administering an effective amount of a VEGF-c, or a variant thereof, or a polynucleotide encoding the VEGF, or the variant thereof, to a meningeal space of the subject, wherein the VEGF-c, or the variant thereof, increases the fluid flow in the central nervous system of the subject; and administering an effective amount of a neurological therapeutic agent for the neurological disease to the subject, thereby treating the Alzheimer's disease.

In still another aspect, the present invention provides a method of reducing an amyloid beta plaque in a subject in need thereof. The method includes administering a VEGF-c, or a variant thereof, a polynucleotide encoding the VEGF, or the variant thereof, to a meningeal space of the subject, wherein the VEGF-c, or the variant thereof increases the fluid flow in the central nervous system of the subject; and administering an effective amount of neurological therapeutic agent to the subject, thereby reducing the amyloid beta plaque aggregate of the protein or peptide in the subject. In one embodiment, the amyloid beta plaque is located in the in the meninges of the subject's brain. In one embodiment, the amyloid beta plaque is reduced by at least about 5%, about 10%, about 15%, about 20%, about 50%, about 1 fold, about 2 fold, or more. In one embodiment, the reduction in the amyloid beta plaque is measured by the number of the amyloid beta plaques, the average size of the amyloid plaques, the coverage of the amyloid beta plaques in a tissue section as the percentage of the area covered by amyloid beta plaques to the area of a tissue section, or any combination thereof. In another embodiment, the coverage of the amyloid beta plaques is the percentage of the area of the amyloid beta plaques to the area of a brain cross section. In still another embodiment, the reduction in the amyloid beta plaques is observed and measured by in vivo magnetic resonance imaging. In yet another embodiment, the method reduces the amyloid beta plaques in the brain of the subject.

In yet another aspect, the present invention provides a method of reducing a microglial inflammatory response in the central nervous system of a subject having an amyloid beta plaque. The method includes administering an effective amount of a flow modulator to a meningeal space of the subject, wherein the flow modulator increases the fluid flow in the central nervous system of the subject; and administering an effective amount of a neurological therapeutic agent to the subject, thereby reducing the microglial inflammatory response in the central nervous system of the subject having the amyloid beta plaque. In one embodiment, the reduction of the microglial inflammatory response include reduction of the number of the microglia surrounding the amyloid plaque, reduction of the percentage of the activated microglia to the total number of microglia, or both. In another embodiment, the methods reduces the microglial inflammatory response by at least about 5%, about 10%, about 15%, about 20%, about 50%, about 99%, or more. In still another embodiment, the methods reduces the microglia inflammatory response as measured by the reduction of the number of microglia surrounding the amyloid beta plaque, reduction of the percentage of activated microglia to total microglia, or both. In one embodiment, the quantity of microglia is measured by measuring the microglia marker IBA. In another embodiment, the level of activated microglia is measured by measuring the activated microglial cell marker CD68. In still another embodiment, the method reduces the microglial inflammatory response in the brain of the subject having the amyloid beta plaque.

In still another aspect, the present invention provides a method of reducing a neurite dystrophy in the central nervous system of a subject having an amyloid beta plaque. The method includes administering an effective amount of a flow modulator to a meningeal space of the subject, wherein the flow modulator increases the fluid flow in the central nervous system of the subject; and administering an effective amount of a neurological therapeutic agent for the neurological disease to the subject, thereby reducing the neurite dystrophy treating the neurological disease in the central nervous system of the subject having the amyloid beta plaque. In one embodiment, the method reduces neurite dystrophy by at least about 5%, about 10%, about 15%, about 20%, about 50%, about 90%, or about 99%. In another embodiment, the neurite dystrophy is measured by the levels of lysosomal-associated membrane protein 1. In still another embodiment, the method reduces the neurite dystrophy in the brain of the subject having the amyloid beta plaque.

In one embodiment of various aspects of the invention, neurological therapeutic agent is an antibody or an antigen binding fragment thereof that specifically binds to amyloid precursor or amyloid beta. In another embodiment, the antibody or the antigen binding fragment thereof is selected from the group consisting of bapineuzumab, gantenerumab, aducanumab, solanezumab, immunoglobulin, BAN2401, semorinemab, zagotenemab, crenezumab, and the antigen binding fragment thereof.

In one embodiment of various aspects of the invention, the VEGF-c, or a variant thereof comprises a polynucleotide comprising a sequence encoding VEGF-c protein, or a variant protein thereof. In another embodiment, the polynucleotide is comprised in an expression vector. In still another embodiment, the expression vector comprises a viral vector. In yet another embodiment, the viral vector comprises an adeno associated virus vector.

In one embodiment of various aspects of the invention, the method of the present invention further include determining the subject to have the neurological disease, a risk factor therefor, or both. In another embodiment, the neurological disease is an Alzheimer's disease and the risk factor is selected from the group consisting of: diploidy for apolipoprotein-E-epsilon-4 (apo-E-epsilon-4), a variant in apo-J, a variant in phosphatidylinositol-binding clathrin assembly protein (PICALM), a variant in complement receptor 1 (CR3), a variant in CD33 (Siglee-3), or a variant in triggering receptor expressed on myeloid cells 2 (TREM2), age, familial AD, a symptom of dementia, or a combination of any of the listed risk factors.

In one aspect, the present invention provides a composition or product combination for a treating a neurological disease. The composition or product combination includes an effective amount of a flow modulator; and an effective amount of a neurological therapeutic agent.

In one embodiment, the flow modulator comprises a lymphangiogenic growth factor or a polynucleotide comprising a sequence encoding the lymphangiogenic growth factor. In another embodiment, the lymphangiogenic growth factor comprises a VEGFR3 agonist or a FGF2, or a variant thereof. In still another embodiment, the VEGFR3 agonist comprises a VEGF-c, a VEGF-d, or a variant thereof. In yet another embodiment, the VEGFR3 agonist comprises a VEGF-c, or a variant thereof. In one embodiment, the VEGF-c variant comprises a VEGFC156S, or a VEGF-165

In another embodiment, the neurological therapeutic agent is selected from the group consisting of a small molecule, a nucleic acid, a peptide, a protein, an antibody, a recombinant virus, a cell, and any combination thereof.

In still another embodiment, the neurological therapeutic agent comprises a small molecule agent. In yet another embodiment, the small molecule agent comprises any one or more agents in Table 2. In yet another embodiment, the small molecule agent is selected from the group consisting of Donepezil, Galantamine, Rivastigmine, Memantine, Lanabecestat, Atabecestat, Verubecestat, Elenbecestat, Semagacestat, Tarenflurbil, and Brexipiprazole.

In one embodiment, the neurological therapeutic agent comprises an antibody or an antigen binding fragment thereof. In another embodiment, the composition or the product combination increases the clearance of a molecule or reduces an aggregate of a protein or peptide, and wherein the antibody or the antigen binding fragment thereof binds to the molecule or the protein or the peptide. In one embodiment, the clearance of a molecule from the central nervous system in a subject is increased by at least about 5%, about 10%, about 15%, about 20%, about 50%, about 100%, about 2 fold or more. In another embodiment, the clearance of a molecule is measured by the amount of the molecule drained into the deep cervical lymph nodes. In one embodiment, the aggregate of the protein or peptide is reduced by at least about 5%, about 10%, about 15%, about 20%, about 50%, or about 100%. In one embodiment, the reduction in the aggregate of the protein or peptide is measured by the reduction in the number of the aggregates, the average size of the aggregates, the coverage of the aggregate in a tissue section as the percentage of the area of the aggregate to the area of a tissue section, or any combination thereof. In another embodiment, the coverage of the aggregates is the percentage of the area covered by the aggregates to the area of a brain cross section. In still another embodiment, the reduction in the aggregates of the protein or peptide is observed and measured by in vivo magnetic resonance imaging.

In another embodiment, the molecule or the protein or the peptide is selected from the group consisting of amyloid precursor protein, amyloid beta, fibrin, tau, apolipoprotein E (Apoe), alpha-synuclein, TDP43, and huntingtin.

In still another embodiment, the protein is amyloid precursor protein or amyloid beta and the antibody or the antigen binding fragment thereof is selected from the group consisting of: bapineuzumab, gantenerumab, aducanumab, solanezumab, immunoglobulin, BAN2401, semorinemab, zagotenemab, crenezumab, and the antigen binding fragment thereof.

In yet another embodiment, the protein is tau and the antibody or the antigen binding fragment thereof is selected from the group consisting of Gosuranemab, Armanezumab, ABBV-8E12 (AbbVie), PHF1, MC1, DA31, 4E6G7, 6B2G12, TOMA, PHF6, PHF13, HJ9.3, HJ9.4, HJ8.5, 43D, 77E9, AT8, MAb86, pS404 mAb IgG2, pS409-tau, PHF1, Ta9, Ta4, Ta1505, DC8E8, and the antigen binding fragment thereof.

In still another embodiment, the protein is alpha-synuclein and the antibody or the antigen binding fragment thereof is selected from the group consisting of BIIB054 (Biogen), PRX002/RG7935 (Roche), prasinezumab (Roche), PD-1601 (AbbVie), 1H7, 5C1, A1-A6, 9E4, 274, NbSyn87*PEST, NAC32, NAC1, AC14, VH14*PEST, syn303, AB1, Human single-chain Fv D10, D5, syn-O1, syn-O2, syn-O4, mAb47, syn-10H, syn-F1, syn-F2, LS4-2G12, and an antigen binding fragment thereof.

In yet another embodiment, the antibody or the antigen binding fragment thereof is selected from the group consisting of bapineuzumab, gantenerumab, aducanumab, solanezumab, crenezumab, pepinemab, ozanezumab, AT-1501, BIIB054, PRX002, and the antigen binding fragment thereof.

In one embodiment, the flow modulator or the neurological therapeutic agent comprises a polynucleotide comprising a sequence encoding a protein or peptide flow modulator or neurological therapeutic agent. In another embodiment, the polynucleotide is comprised in an expression vector. In still another embodiment, the expression vector is a viral vector. In yet another embodiment, the viral vector is a adeno-associated virus vector.

In another embodiment, the flow modulator comprises a protein or a peptide and the neurological therapeutic agent comprises a protein or a peptide. In still another embodiment, the flow modulator comprises a protein or a peptide and the neurological therapeutic agent comprises a polynucleotide comprising a sequence encoding a protein or a peptide. In yet another embodiment, the flow modulator comprises a polynucleotide comprising a sequence encoding a protein or a peptide and the neurological therapeutic agent comprises a protein or a peptide. In one embodiment, the flow modulator comprises a polynucleotide comprising a sequence encoding a protein or a peptide and the neurological therapeutic agent comprises a sequence encoding a protein or a peptide.

In one embodiment, the flow modulator is different than the neurological therapeutic agent.

In one embodiment, the flow modulator is in an effective amount to increases the diameter of the meningeal lymphatic vessel by at least about 5%, about 10%, about 15%, or about 20%. In one embodiment, the increase in the diameter of the meningeal lymphatic vessel is detected or measured by magnetic resonance imaging.

In another embodiment, the neurological disease is selected from the group consisting of Alzheimer's disease, Parkinson's disease, cerebral edema, amyotrophic lateral sclerosis (ALS), Pediatric Autoimmune Neuropsychiatric Disorders Associated with Streptococcal Infections (PANDAS), meningitis, hemorrhagic stroke, Autism spectrum disorder (ASD), brain tumor, epilepsy, Down's syndrome, hereditary cerebral hemorrhage with amyloidosis-Dutch type (HCHWA-D), Familial Danish/British dementia, dementia with Lewy bodies (DLB), Lewy body (LB) variant of AD, multiple system atrophy (MSA), familial encephalopathy with neuroserpin inclusion bodies (FENIB), frontotemporal dementia (FTD), Huntington's disease (HD), Kennedy disease/spinobulbar muscular atrophy (SBMA), dentatorubropallidoluysian atrophy (DRPLA); spinocerebellar ataxia (SCA) type I, SCA2, SCA3 (Machado-Joseph disease), SCA6, SCA7, SCA17, Creutzfeldt-Jakob disease (CJD), familial CJD, Kuru, Gerstmann-Straussler-Scheinker syndrome (GSS), fatal familial insomnia (FFI), corticobasal degeneration (CBD), progressive supranuclear palsy (PSP), cerebral amyloid angiopathy (CAA), multiple sclerosis (MS), AIDS-related dementia complex, brain injury, traumatic brain injury, brain hemorrhage, subarachnoid brain hemorrhage, stroke, and any combination thereof.

In one embodiment, the flow modulator is formulated for administration to the subject by a route selected from the group consisting of: intrathecal administration, intraventricular administration, intraparenchymal administration, nasal administration, transcranial administration, contact with cerebral spinal fluid (CSF) of the subject, pumping into CSF of the subject, implantation into the skull or brain, contacting a thinned skull or skull portion of the subject with the flow modulator, expression in the subject of a nucleic acid encoding the flow modulator, and any combination thereof.

In another embodiment, the neurological therapeutic agent is formulated for administration to the subject by a route selected from the group consisting of: intrathecal administration, intraventricular administration, intraparenchymal administration, nasal administration, transcranial administration, contact with cerebral spinal fluid (CSF) of the subject, pumping into CSF of the subject, implantation into the skull or brain, contacting a thinned skull or skull portion of the subject with the neurological therapeutic agent, expression in the subject of a nucleic acid encoding the neurological therapeutic agent, intravenous infusion, and any combination thereof.

In still another embodiment, the flow modulator and the neurologic therapeutic agent are formulated in a same pharmaceutic composition. In yet another embodiment, the flow modulator and the neurologic therapeutic agent are formulated in different pharmaceutic composition.

In one aspect, the present invention provides a composition or product combination for a treating an Alzheimer's disease. The composition or product combination includes an effective amount of a VEGF, or a variant thereof, or a polynucleotide encoding the VEGF, or the variant thereof, and an effective amount of a neurological therapeutic agent. In one embodiment, the neurological therapeutic agent is an antibody or an antigen binding fragment thereof that specifically binds to amyloid precursor protein or amyloid beta. In still another embodiment, the antibody or the antigen binding fragment thereof is selected from the group consisting of bapineuzumab, gantenerumab, aducanumab, solanezumab, immunoglobulin, BAN2401, semorinemab, zagotenemab, crenezumab, and the antigen binding fragment thereof.

In one embodiment of various aspects of the invention, the VEGF-c, or a variant thereof comprises a polynucleotide comprising a sequence encoding VEGF-c protein, or a variant protein thereof. In another embodiment, the polynucleotide is comprised in an expression vector. In still another embodiment, the expression vector comprises a viral vector. In yet another embodiment, the viral vector comprises an adeno associated virus vector.

### BRIEF DESCRIPTION OF THE DRAWINGS

FIGS. 1A-1G are a series of microscope images and graphs showing effects of the flow modulator VEGF-c and the neurological therapeutic agent amyloid beta antibody (ABETA Ab) on the meningeal blood and lymphatic vasculature of adult 5×FAD mice in accordance with some embodiments.

FIG. 1C is a graph showing the difference of the transverse sinus diameter between mice that received different treatments. The mice that received ABETA Ab (results pooled from all the mice that received eGFP or VEGF-c) have larger transverse sinus diameter (p value=0.0610) than the mice that receive control mIgG2a (results pooled from all the mice that received eGFP or VEGF-c).

FIG. 1D is a graph showing the percentage coverage of the meningeal blood vasculature (% of field of view) in the different groups.

FIG. 1E is a graph showing the difference of the number of meningeal lymphatic branchings between mice that receive different treatments. The mice that receive ABETA Ab (results pooled from all the mice that receive eGFP or VEGF-c) have larger number of branchings (p value=0.0088) than the mice that receive control mIgG2a (results pooled from all the mice that receive eGFP or VEGF-c).

FIG. 1F is a graph showing the diameter of meningeal lymphatic vasculature in the different groups.

FIG. 1G is a graph showing the coverage by meningeal lymphatic vessels (% of field of view, “FOV”) between mice that receive different treatments. The mice that receive ABETA Ab and VEGF-c have large % FOV than the mice that receive mIgG2 control and eGFP (p value=0.0870). The mice that receive VEGF-c (results pooled from all the mice that receive ABETA Ab or control mIgG2) have larger % FOV than the mice that receive control eGFP mIgG2a (results pooled from all the mice that receive ABETA Ab or mIgG2) (p value=0.0540). The p values in FIGS. 1E and 1G result from the two-way ANOVA with multiple comparisons test.

FIGS. 2A-2M are a series of fluorescence microscopy images and graphs showing effects of the flow modulator VEGF-c and the neurological therapeutic agent amyloid beta antibody on brain amyloid beta plaques in adult 5×FAD mice in accordance with some embodiments.

FIG. 2B is a graph showing the quantifications of amyloid beta plaque density in the brain cortex, striatum and amygdala in the different groups.

FIG. 2C is a graph showing the differences in the average size of Aβ plaques in the cortex, striatum and amygdala between mice that received different treatments. The mice that received ABETA Ab and VEGF-c have smaller average size of Aβ plaque than the mice that receive mIgG2a and eGFP (p value=0.0272). The mice that receive VEGF-c (results pooled from all the mice that receive mIgG2 or ABETA Ab) have smaller average size of Aβ plaque than the mice that receive eGFP (results pooled from all the mice that receive mIgG2 or ABETA Ab) (p value=0.0340).

FIG. 2D is a graph showing the differences in the coverage of Aβ plaques (% of brain section) in the cortex, striatum and amygdale between mice that received different treatments. The mice that receive ABETA Ab and VEGF-c have smaller coverage of Aβ plaque than the mice that receive mIgG2a and eGFP (p value=0.0084). The mice that receive ABETA AB (results pooled from all the mice that receive VEGF-c or eGFP) have smaller coverage of Aβ plaque than the mice that receive mIgG2 control (results pooled from all the mice that receive VEGF-c or eGFP) (p value=0.0520). The mice that receive VEGF-c (results pooled from all the mice that receive mIgG2 or ABETA Ab) have smaller coverage of Aβ plaque than the mice that receive eGFP (results pooled from all the mice that receive mIgG2 or ABETA Ab) (p value=0.0156).

FIG. 2E is a graph showing the quantifications of amyloid beta plaque density in the brain hippocampus in the different groups.

FIG. 2F is a graph showing the differences in the average size of Aβ plaques in the hippocampus between mice that received different treatments. The mice that received ABETA Ab and VEGF-c have smaller average size of Aβ plaque than the mice that receive mIgG2a and eGFP (p value=0.0398). The mice that received ABETA AB (results pooled from all the mice that receive VEGF-c or eGFP) have smaller average size of Aβ plaque than the mice that receive mIgG2 control (results pooled from all the mice that receive VEGF-c or eGFP) (p value=0.0364).

FIG. 2G is a graph showing the differences in the coverage of Aβ plaques (% of brain section) in the hippocampus between mice that received different treatments. The mice that receive ABETA Ab and VEGF-c have smaller coverage of Aβ plaque than the mice that receive mIgG2a and eGFP (p value=0.0282). The mice that receive ABETA AB (results pooled from all the mice that receive VEGF-c or eGFP) have smaller coverage of Aβ plaque than the mice that receive mIgG2 control (results pooled from all the mice that receive VEGF-c or eGFP) (p value=0.0583). The mice that receive VEGF-c (results pooled from all the mice that receive mIgG2 or ABETA Ab) have smaller coverage of Aβ plaque than the mice that receive eGFP (results pooled from all the mice that receive mIgG2 or ABETA Ab) (p value=0.0635).

FIG. 2H is a graph showing the quantifications of amyloid beta plaque density in the brain thalamus and hypothalamus in the different groups.

FIG. 2I is a graph showing the differences in the average size of Aβ plaques in the thalamus and hypothalamus between mice that received different treatments. The mice that received ABETA Ab and VEGF-c have smaller average size of Aβ plaque than the mice that receive mIgG2a and eGFP (p value=0.0214). The mice that received ABETA AB (results pooled from all the mice that receive VEGF-c or eGFP) have smaller average size of Aβ plaque than the mice that receive mIgG2 control (results pooled from all the mice that receive VEGF-c or eGFP) (p value=0.0667). The mice that received VEGF-c (results pooled from all the mice that receive mIgG2 or ABETA Ab) have smaller average size of Aβ plaque than the mice that received eGFP (results pooled from all the mice that receive mIgG2 or ABETA Ab) (p value=0.0392).

FIG. 2J is a graph showing the differences in the coverage of Aβ plaques (% of brain section) in the thalamus and hypthalamus between mice that received different treatments. The mice that receive ABETA Ab and VEGF-c have smaller coverage of Aβ plaque than the mice that receive mIgG2a and eGFP (p value=0.0267). The mice that receive ABETA AB (results pooled from all the mice that receive VEGF-c or eGFP) have smaller coverage of Aβ plaque than the mice that receive mIgG2 control (results pooled from all the mice that receive VEGF-c or eGFP) (p value=0.0713). The mice that receive VEGF-c (results pooled from all the mice that receive mIgG2 or ABETA Ab) have smaller coverage of Aβ plaque than the mice that receive eGFP (results pooled from all the mice that receive mIgG2 or ABETA Ab) (p value=0.0483).

FIG. 2K is a graph showing the quantifications of amyloid beta plaque density in the whole brain tissue section.

FIG. 2L is a graph showing the differences in the average size of Aβ plaques in the whole brain between mice that receive different treatments. The mice that receive ABETA Ab and VEGF-c have smaller average size of Aβ plaque than the mice that receive mIgG2a and eGFP (p value=0.0226). The mice that receive ABETA AB (results pooled from all the mice that receive VEGF-c or eGFP) have smaller average size of Aβ plaque than the mice that receive mIgG2 control (results pooled from all the mice that receive VEGF-c or eGFP) (p value=0.0529). The mice that receive VEGF-c (results pooled from all the mice that receive mIgG2 or ABETA Ab) have smaller average size of Aβ plaque than the mice that receive eGFP (results pooled from all the mice that receive mIgG2 or ABETA Ab) (p value=0.0535).

FIG. 2M is a graph showing the differences in the coverage of Aβ plaques (% of brain section) in the whole brain between mice that received different treatments. The mice that receive ABETA Ab and VEGF-c have smaller coverage of Aβ plaque than the mice that receive mIgG2a and eGFP (p value=0.0152). The mice that receive ABETA AB (results pooled from all the mice that receive VEGF-c or eGFP) have smaller coverage of Aβ plaque than the mice that receive mIgG2 control (results pooled from all the mice that receive VEGF-c or eGFP) (p value=0.0523). The mice that receive VEGF-c (results pooled from all the mice that receive mIgG2 or ABETA Ab) have smaller coverage of Aβ plaque than the mice that receive eGFP (results pooled from all the mice that receive mIgG2 or ABETA Ab) (p value=0.0331). The p values in FIGS. 2C, 2D, 2F, 2G, 2I, 2J, 2L, and 2M results from the two-way ANOVA with multiple comparisons test.

FIGS. 3A-3G are a series of graphs and fluorescence microscopy images showing effects of the flow modulator VEGF-c and the neurological therapeutic agent amyloid beta antibody on behavior and amyloid beta plaques of aged APPswe mice in accordance with some embodiments. Behaviors assessed include open field, novel location recognition, and contextual fear conditioning.

FIGS. 4A-4D are a series of graphs and microscope images showing effects of the flow modulator VEGF-c and the neurological therapeutic agent amyloid beta antibody on amyloid beta plaques of J20 mice in accordance with some embodiments.

FIGS. 5A-5Q are flow cytometry dot plots and histograms showing the quantification of number or percentage of different meningeal immune cell populations in 3 months-old 5×FAD mice and WT age-matched littermate controls.

FIG. 5A is representative flow cytometry dot plots showing gating strategy for CD64⁺ macrophages (pre-gated in singlets CD45⁺ live cells), CD19⁺ B cells (pre-gated in CD64^{neg} cells) and CD11c⁺MHC-II^{high} dendritic cells (DCs, pre-gated in CD19^{neg} cells).

FIG. 5B is representative flow cytometry dot plots showing gating strategy for γδT cells (pre-gated in singlets CD45⁺ live cells), TCRβ^{neg}NK1.1⁺ cells (NK cells, pre-gated in TCR^{neg} cells), TCRβ⁺NK1.1⁺ cells (NKT cells, pre-gated in TCRβ⁺ cells) and CD4⁺ and CD8⁺ T cells (pre-gated in TCRβ⁺NK1.1^{neg} cells).

FIGS. 5C-5K show number of total CD45⁺ live (FIG. 5C), CD64⁺ macrophages (FIG. 5D), B cells (FIG. 5E), DCs (FIG. 5F), γδT cells (FIG. 5G), NK cells (FIG. 5H), NKT cells (FIG. 5I), CD4⁺ T cells (FIG. 5J) and CD8⁺ T cells (FIG. 5K) isolated from the meninges of 3 months-old wild type (WT, blue) and 5×FAD (green) mice. FIG. 5L is histograms showing the expression levels of PD-1 in the fluorescence minus one (FMO) sample (grey) or in concatenated meningeal immune cell populations from WT (blue) and 5×FAD (green) groups. Cells considered to be positive for PD-1 are demarcated in the different histograms.

FIGS. 5M-5Q show frequencies of PD-1-expressing γδT cells (FIG. 5M), NK cells (FIG. 5N), NKT cells (FIG. 5O), CD4⁺ T cells (FIG. 5P) and CD8⁺ T cells (FIG. 5Q) in each group. Results in FIGS. 5C-5K and FIGS. 5M-5Q are presented as mean±s.e.m.; n=5 per group; two-tailed unpaired Student's T test; data is representative of 2 independent experiments.

FIGS. 6A-6Q show compromised meningeal lymphatic function is observed in middle-aged 5×FAD mice and that meningeal lymphatic ablation (using a pharmacological method) in 5×FAD mice limits brain Aβ clearance by anti-Abeta antibody.

FIG. 6A is representative images of meningeal whole mounts from 3-, 6- and 14-month-old 5×FAD mice, stained for CD31 (blue), LYVE-1 (green) and Aβ (red; scale bar, 2 mm; inset scale bar, 400 μm).

FIGS. 6B-6E are graphs showing the coverage by LYVE-1+CD31⁺ vessels (in mm²) and the number of branching points in lymphatics at the superior sagittal sinus (SSS) (FIGS. 6B and 6C) and at the confluence of sinuses (COS), transverse (TS), sigmoid (SS) and petrosquamosal (PSS) sinuses (FIGS. 6D and 6E).

FIG. 6F shows total coverage by Aβ (% of meningeal whole mount). Results in FIGS. 6B-6F are presented as mean±s.e.m.; n=4 per group; One-way ANOVA with Sidak's multiple comparisons test; data in FIGS. 6B-6F results from 2 independent experiments.

FIG. 6G shows adult 2 months-old WT mice were injected (i.c.m.) with Visudyne (5 L) followed by a transcranial photoconversion step (Vis./photo.) to ablate meningeal lymphatic vessels. Control mice were injected with Visudyne without photoconversion (Vis.). One week later, mice were injected with 5 μL of a suspension of fluorescent microspheres (1 μm in diameter) into the CSF and 15 minutes later the lymphatic vessel afferent to the deep cervical lymph node (dCLN) was imaged by in vivo stereomicroscopy. Representative images of skull cap showing microspheres (blue) and lymphatic vessels stained for LYVE-1 (green) around the confluence of sinuses (COS) and transverse sinus (TS) at the dorsal brain meninges or around the sigmoid (SS) and petrosquamosal (PSS) sinuses at the basal brain meninges (scale bars, 500 μm).

FIGS. 6H and 6I are graphs showing LYVE-1⁺ vessel total length (in mm) and branching points in dorsal meninges (FIG. 6H) and basal meninges (FIG. 6I). Results in FIGS. 6H and 6I are presented as mean±s.e.m.; n=10 per group; two-tailed unpaired Student's T test; data is representative of 2 independent experiments.

FIG. 6J is representative frames showing microspheres flowing through the lymphatic vessel afferent to the dCLN, or cumulative sphere tracking (for 20 sec), in mice with intact or ablated meningeal lymphatic vessels.

FIG. 6K is graph with quantifications of microsphere flow (number microspheres per minute) in mice from different groups. Results in FIG. 6K are presented as mean±s.e.m.; n=11 in Vis. group and n=14 in Vis./photo. group; two-tailed unpaired Student's T test; data results from 2 independent experiments.

FIG. 6L shows that adult 4-5 months-old female 5×FAD mice were injected (i.c.m.) with Visudyne (5 μL) plus photoconversion (Vis./photo.) or Visudyne without photoconversion (Vis.). One week later, mice were injected (i.c.m.) with ABETA Ab (murine antibody against Aβ protofibrils) or murine IgG2a (mIgG2a) as a control (both at 1 μg/L). Injection of anti-Abeta antibody or mIgG2a was repeated two weeks later. Another lymphatic vessel ablation step was performed, followed by two injections with ABETA Ab or mIgG2a.

FIG. 6M is representative images of brain sections from 5×FAD mice stained for Aβ (red) and with DAPI (blue; scale bar, 1 mm).

FIGS. 6N-6Q are graphs showing number of Aβ plaques per mm², average size of Aβ plaques (μm²) and coverage of Aβ (% of region) in the hippocampus (FIG. 6N), the cortex/striatum/amygdala (FIG. 6O), the thalamus/hypothalamus (FIG. 6P) and the whole brain sections (FIG. 6Q). Results in FIGS. 6N-6Q are presented as mean±s.e.m.; n=5 in mIgG2a groups and n=6 in anti-Abeta antibody groups; Two-way ANOVA with Sidak's multiple comparisons test.

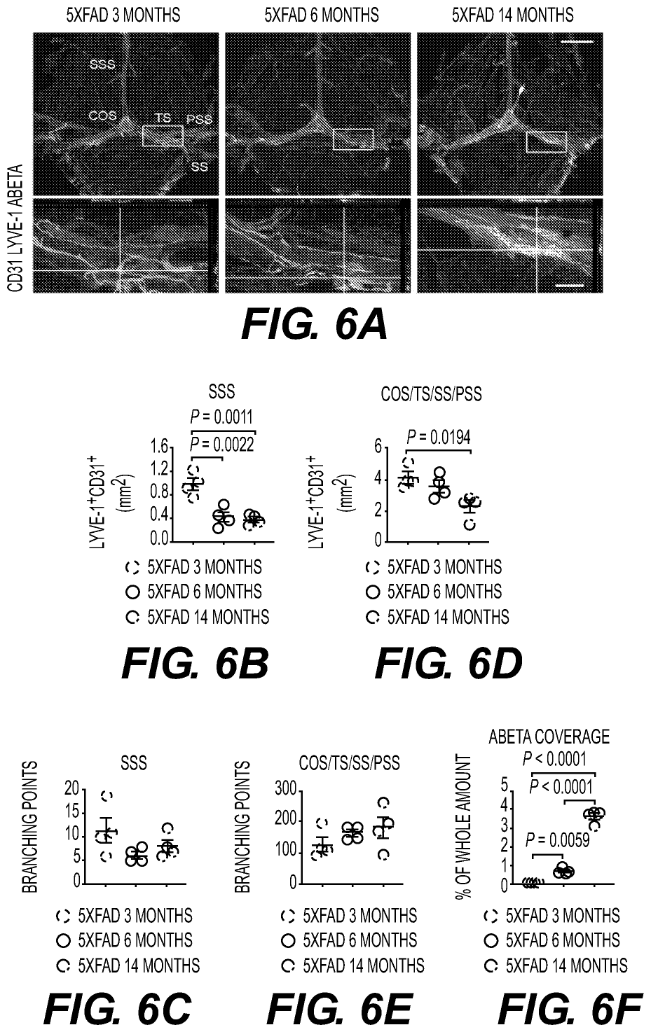

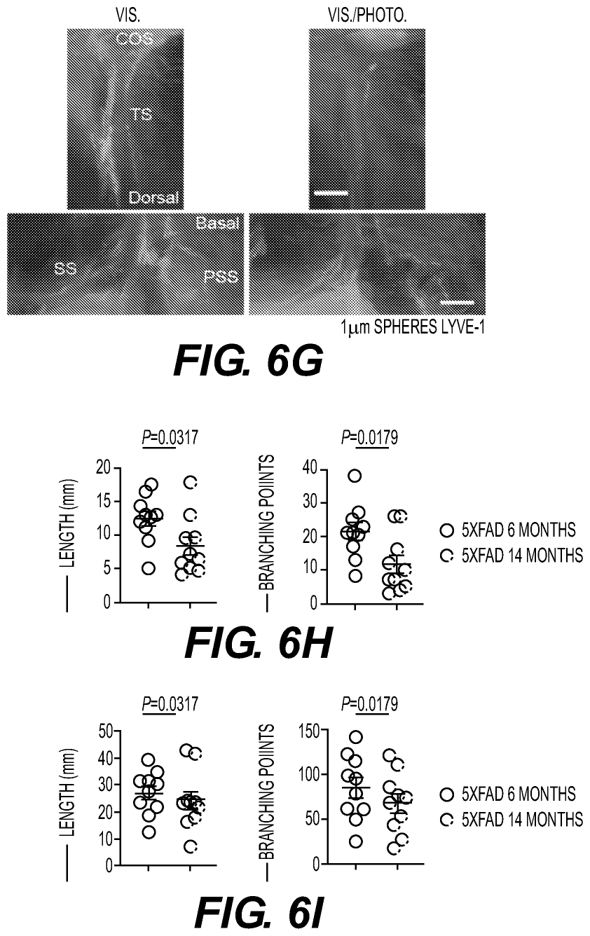

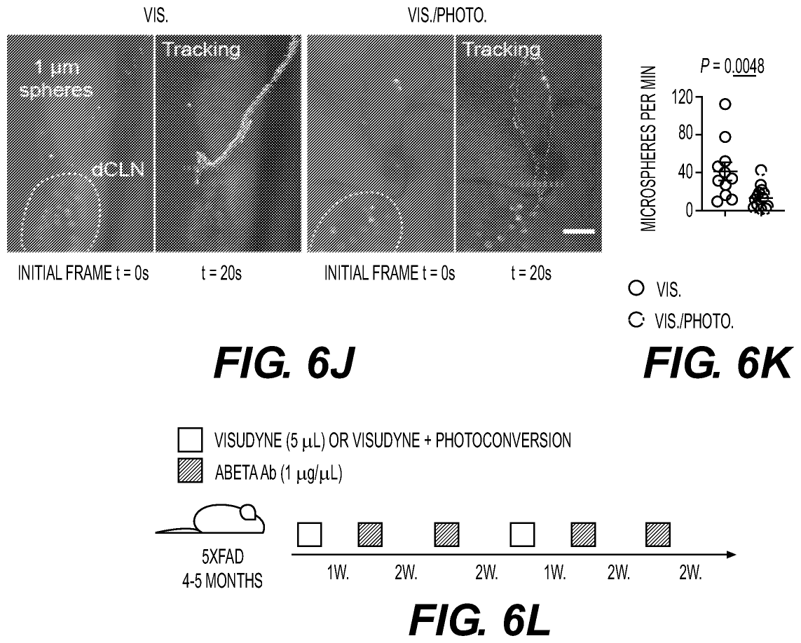

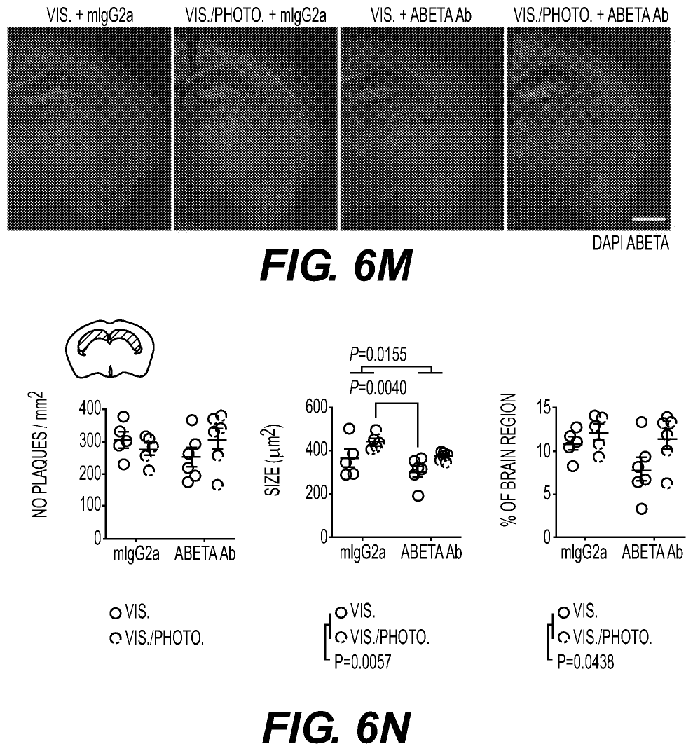

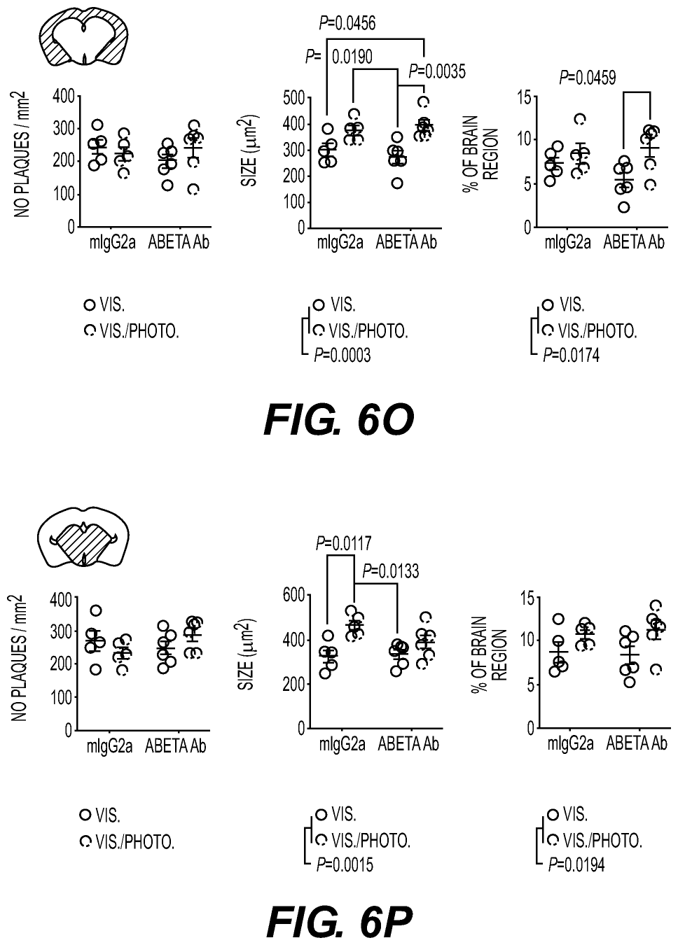

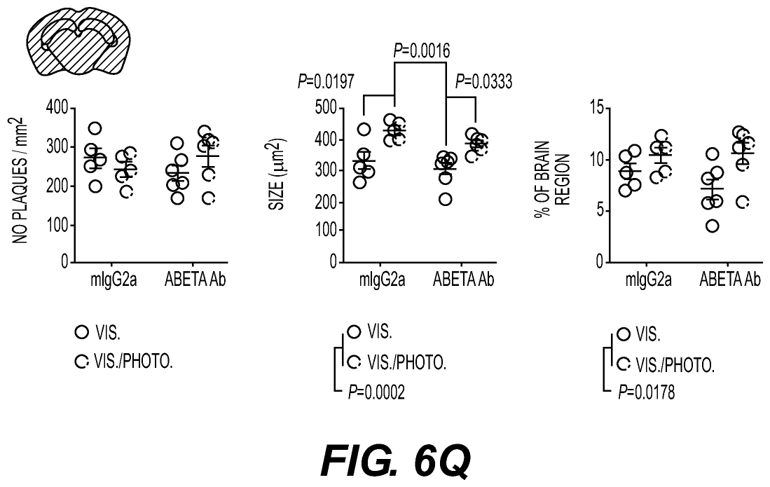

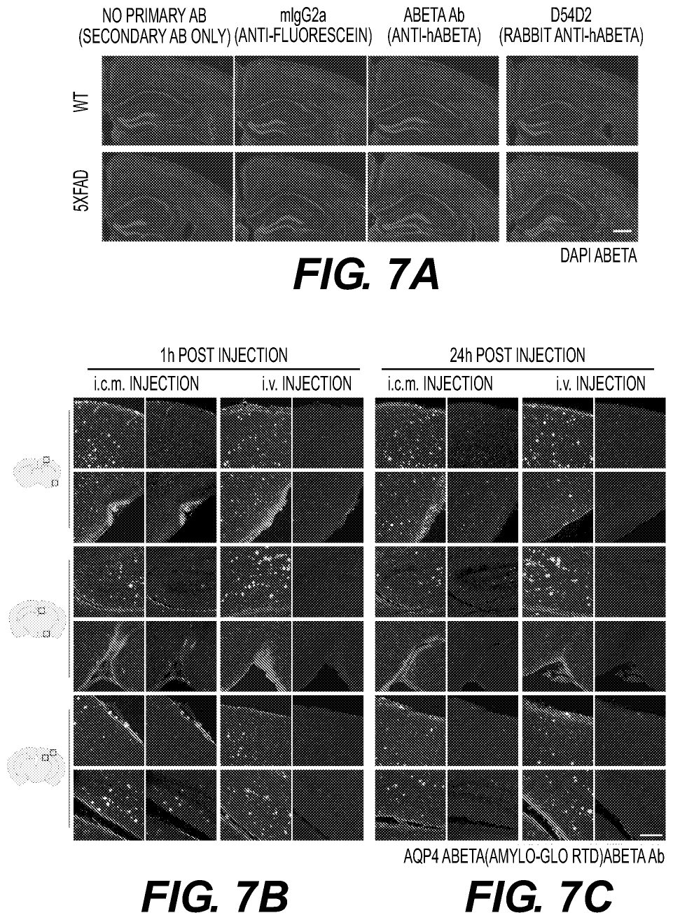

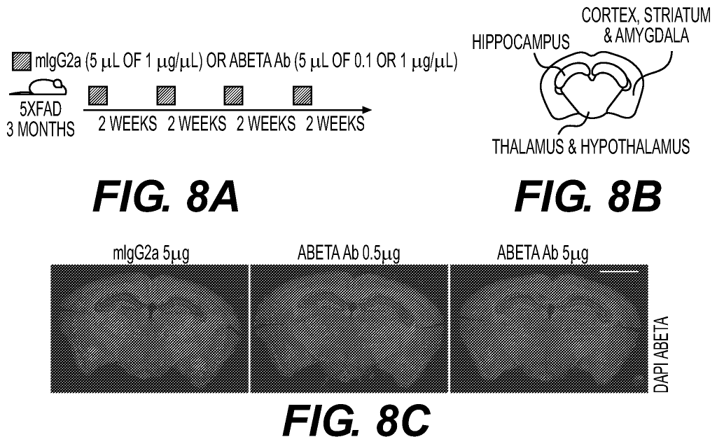

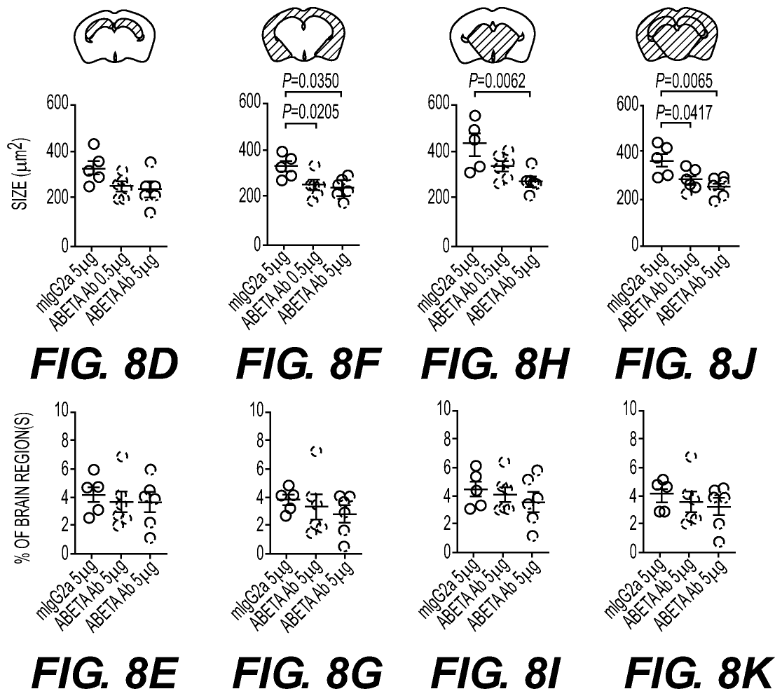

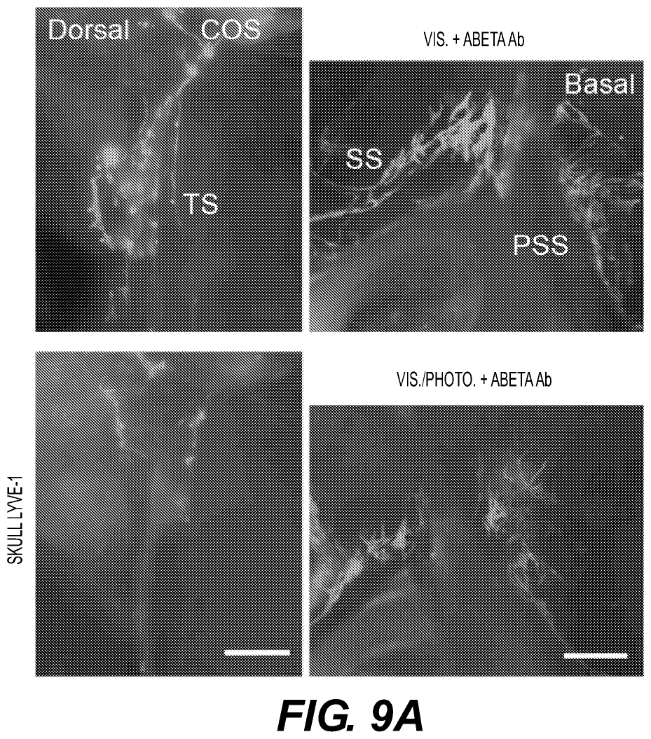

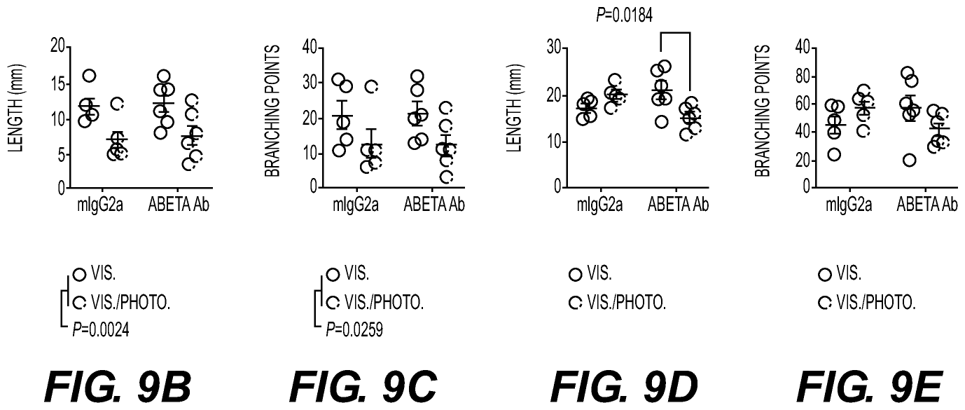

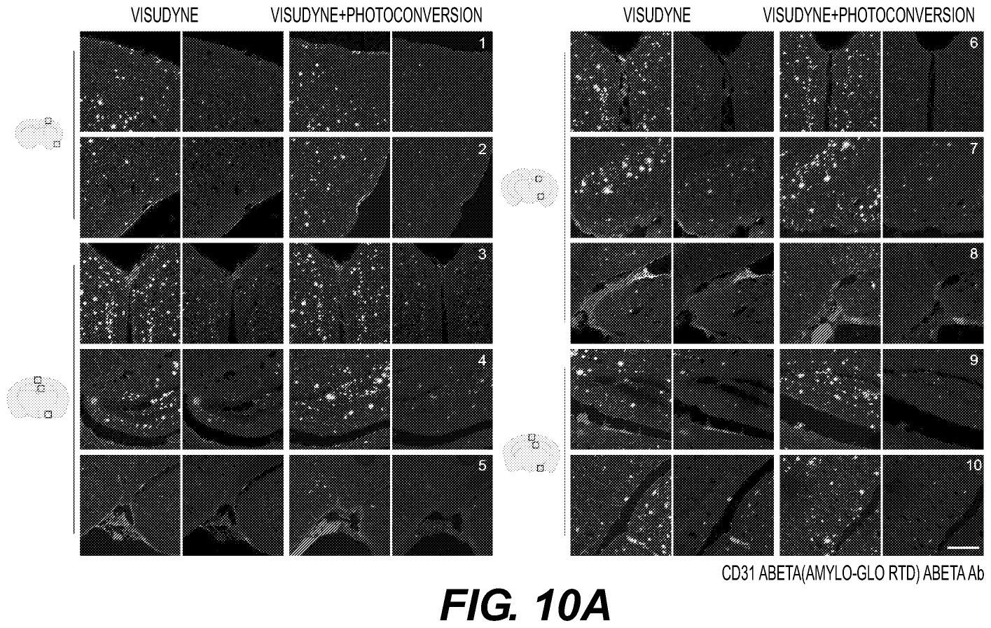

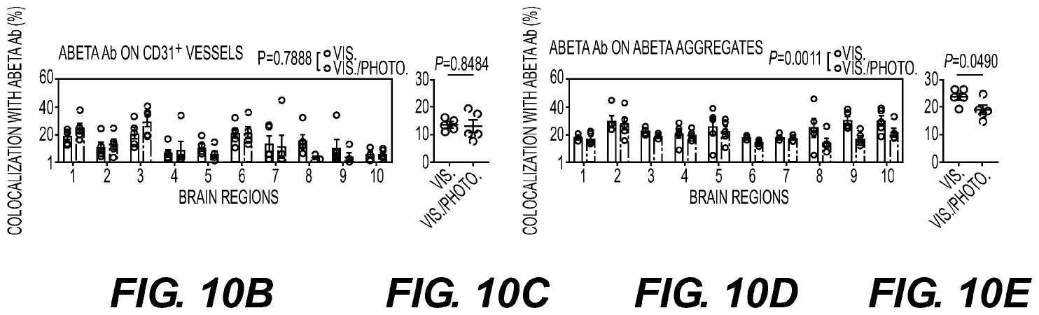

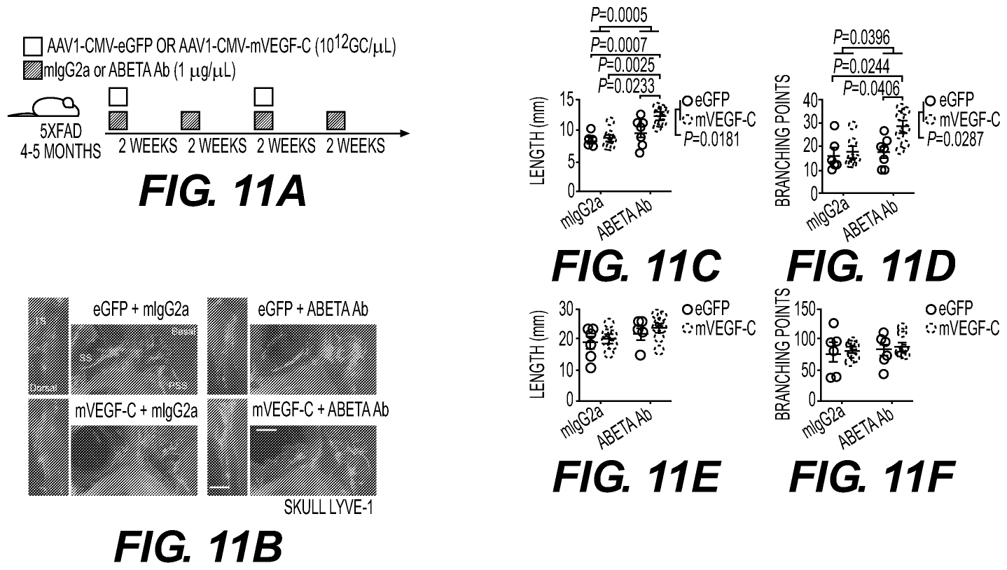

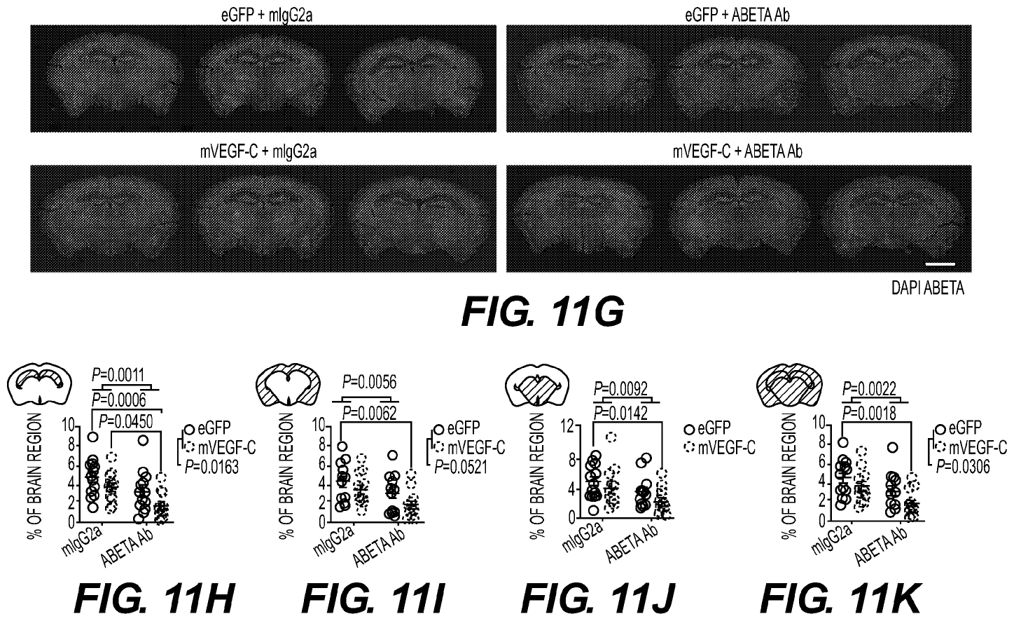

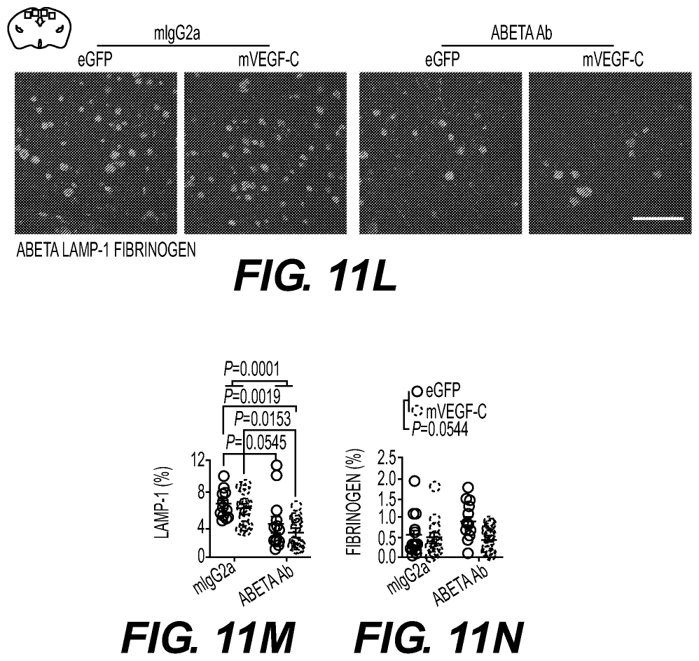

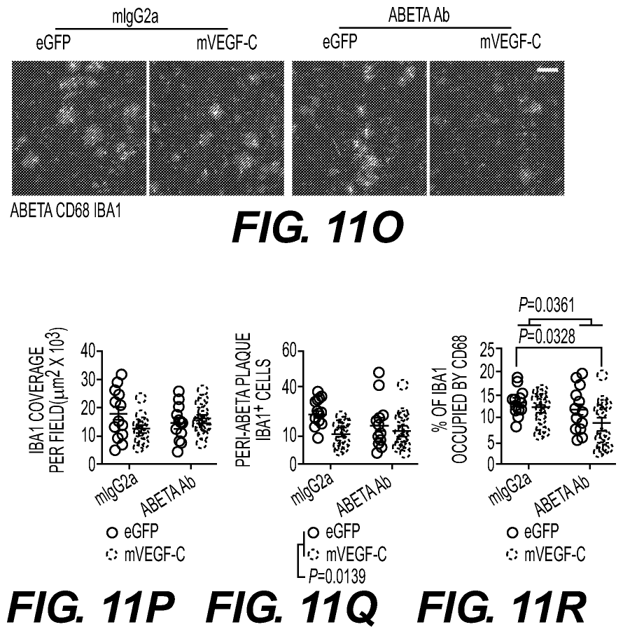

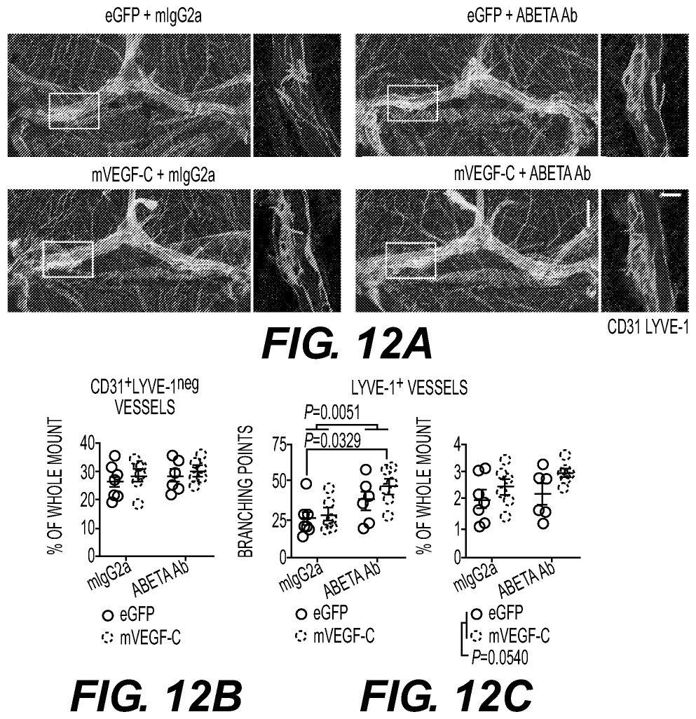

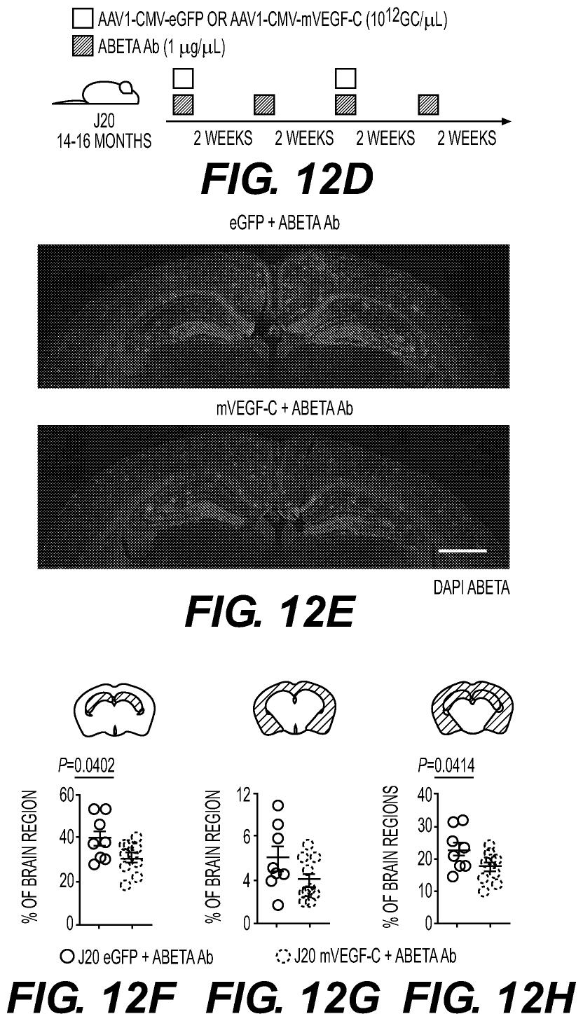

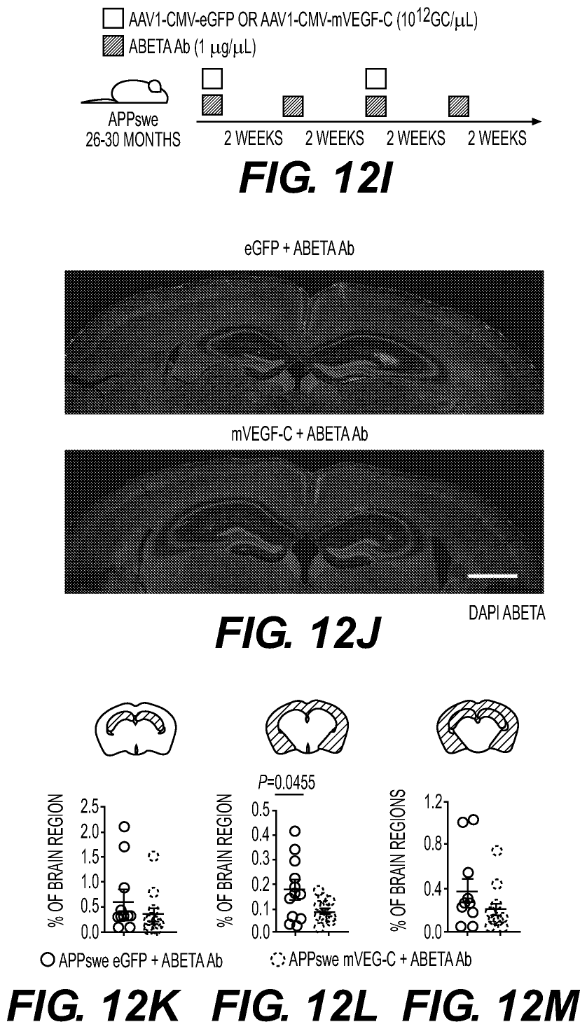

The middle panel of FIG. 6N is a graph showing the differences in the average size of Aβ plaques (μm²) in the hippocampus between mice that received different treatments. The mice that were treated with Visudyne and ABETA Ab have smaller average size of Aβ plaques (μm²) than the mice that were treated with Visudyne and photoconversion and mIgG2a (p value=0.0040). The mice that were treated with ABETA Ab (results pooled from all the mice that were treated with Visudyne or Visudyne and photoconversion) have smaller average size of Aβ plaques (μm²) than the mice that were treated with mIgG2 (results pooled from all the mice that were treated with Visudyne or Visudyne and photoconversion) (p value=0.0155). The mice that were treated with Visudyne (results pooled from all the mice that were treated with ABETA Ab or mIgG2) have smaller average size of Aβ plaques (μm²) than the mice that were treated with Visudyne and photoconversion (results pooled from all the mice that were treated with ABETA Ab or mIgG2) (p value=0.0057). In a comparison between two groups, the mice that were treated with Visudyne and ABETA Ab have smaller average size of Aβ plaques (μm²) than the mice that were treated with Visudyne and photoconversion and ABETA (p value=0.0227, obtained by T test and not shown in FIG. 6N).

The right panel of FIG. 6N is a graph showing the differences in the coverage of Aβ plaques (% of brain region) in the hippocampus between mice that received different treatments. The mice that were treated with Visudyne (results pooled from all the mice that were treated with ABETA Ab or mIgG2) have smaller average size of Aβ plaques (μm²) than the mice that were treated with Visudyne and photoconversion (results pooled from all the mice that were treated with ABETA Ab or mIgG2) (p value=0.0438).

The middle panel of FIG. 6O is a graph showing the differences in the average size of Aβ plaques (μm²) in the cortex, striatum and amygdala between mice that received different treatments. The mice that were treated with Visudyne and ABETA Ab have smaller average size of Aβ plaques (μm²) than the mice that were treated with Visudyne and photoconversion and mIgG2a (p value=0.0190). The mice that were treated with Visudyne and ABETA Ab have smaller average size of Aβ plaques (μm²) than the mice that were treated with Visudyne and photoconversion and ABETA Ab (p value=0.0035). The mice that were treated with Visudyne and mIgG2a have smaller average size of Aβ plaques (μm²) than the mice that were treated with Visudyne and photoconversion and ABETA Ab (p value=0.0456). The mice that were treated with Visudyne (results pooled from all the mice that were treated with ABETA Ab or mIgG2) have smaller average size of Aβ plaques (μm²) than the mice that were treated with Visudyne and photoconversion (results pooled from all the mice that were treated with ABETA Ab or mIgG2) (p value=0.0003).

The right panel of FIG. 6O is a graph showing the differences in the coverage of Aβ plaques (% of brain region) in the cortex, striatum and amygdala between mice received different treatments. The mice that were treated with Visudyne and ABETA Ab have smaller coverage of Aβ plaques than the mice that were treated with Visudyne and photoconversion and ABETA Ab (p value=0.0459). The mice that were treated with Visudyne (results pooled from all the mice that were treated with ABETA Ab or mIgG2) have smaller coverage of Aβ plaques than the mice that were treated with Visudyne and photoconversion (results pooled from all the mice that were treated with ABETA Ab or mIgG2) (p value=0.0174).

The middle panel of FIG. 6P is a graph showing the differences in the average size of Aβ plaques (μm²) in the thalamus and hypothalamus between mice received different treatments. The mice that were treated with Visudyne and ABETA Ab have smaller average size of Aβ plaques (μm²) than the mice that were treated with Visudyne and photoconversion and mIgG2a (p value=0.0133). The mice that were treated with Visudyne and mIgG2 have smaller average size of Aβ plaques (μm²) than the mice that were treated with Visudyne and photoconversion and mIgG2 (p value=0.0117). The mice that were treated with Visudyne (results pooled from all the mice that were treated with ABETA Ab or mIgG2) have smaller average size of Aβ plaques (μm²) than the mice that were treated with Visudyne and photoconversion (results pooled from all the mice that were treated with ABETA Ab or mIgG2) (p value=0.0015).

The right panel of FIG. 6P is a graph showing the differences in the coverage of Aβ plaques (% of brain region) in the thalamus and hypothalamus between mice received different treatments. The mice that were treated with Visudyne (results pooled from all the mice that were treated with ABETA Ab or mIgG2) have smaller coverage of Aβ plaques than the mice that were treated with Visudyne and photoconversion (results pooled from all the mice that were treated with ABETA Ab or mIgG2) (p value=0.0194).

The middle panel of FIG. 6Q is a graph showing the differences in the average size of Aβ plaques (μm²) in the whole brain between mice received different treatments. The mice that were treated with Visudyne and ABETA Ab have smaller average size of Aβ plaques (μm²) than the mice that were treated with Visudyne and photoconversion and mIgG2a (p value=0.0016). The mice that were treated with Visudyne and mIgG2 have smaller average size of Aβ plaques (μm²) than the mice that were treated with Visudyne and photoconversion and mIgG2 (p value=0.0197). The mice that were treated with Visudyne and ABETA have smaller average size of Aβ plaques (μm²) than the mice that were treated with Visudyne and photoconversion and ABETA Ab (p value=0.0333). The mice that were treated with Visudyne (results pooled from all the mice that were treated with ABETA Ab or mIgG2) have smaller average size of Aβ plaques (μm²) than the mice that were treated with Visudyne and photoconversion (results pooled from all the mice that were treated with ABETA Ab or mIgG2) (p value=0.0002).

The right panel of FIG. 6Q is a graph showing the differences in the coverage of Aβ plaques (% of brain region) in the whole brain between mice received different treatments. The mice that were treated with Visudyne (results pooled from all the mice that were treated with ABETA Ab or mIgG2) have smaller coverage of Aβ plaques than the mice that were treated with Visudyne and photoconversion (results pooled from all the mice that were treated with ABETA Ab or mIgG2) (p value=0.0178). In a comparison between two groups, the mice that were treated with Visudyne and ABETA Ab have smaller coverage of Aβ plaques than the mice that were treated with Visudyne and photoconversion and ABETA Ab (p value=0.0057, obtained by T test and not shown in FIG. 6Q). The p values in FIGS. 6N-6Q result from the two-way ANOVA with multiple comparisons test unless otherwise indicated as a comparison between two groups using t test.

FIGS. 7A-7C show specificity of anti-Abeta antibody and kinetics of brain Aβ recognition upon i.c.m. or i.v. injections.

FIG. 7A is representative images of brain sections from 4 months-old 5×FAD mice and WT littermate controls that were incubated without primary antibody (ab), with murine IgG2a isotype control (anti-fluorescein), anti-Abeta antibody murine IgG2a monoclonal antibody (against human Aβ protofibrils) or a commercially available rabbit anti-human Aβ (D54D2 clone, optimal for immunofluorescence staining). Images show Aβ (red) and DAPI (blue) staining (scale bar, 1 mm).

FIG. 7B is images of different regions of the brain of 5×FAD mice, 1-hour post injection of anti-Abeta antibody (1 μg/L) into the CSF (5 μL, i.c.m.) or into the blood (100 μL, i.v.).

FIG. 7C is images of different regions of the brain of 5×FAD mice 24 hours post injection of anti-Abeta antibody into the CSF (i.c.m.) or into the blood (i.v.). Images in FIG. 7B and FIG. 7C panels show astrocyte endfeet and glia limitans stained for Aquaporin 4 (AQP4, in blue), Aβ stained with Amylo-Glo RTD (green) and anti-Abeta antibody staining (red; scale bar, 200 μm). Data is representative of 2 independent experiments.

FIGS. 8A-8K show repeated delivery of anti-Abeta antibody into the CSF (by cisterna magna injection) of 5×FAD mice reduces brain Aβ plaque load.

FIG. 8A shows that adult 5×FAD mice (3 months-old) were injected (i.c.m.) with either mIgG2a (5 μL at 1 μg/L) or anti-Abeta antibody (5 μL at 0.1 or 1 μg/L). Injections were repeated another three times, every two weeks, as shown in the scheme.

FIG. 8B is representation of different brain regions considered for the quantification of Aβ, namely hippocampus, cortex/striatum/amygdala and thalamus/hypothalamus.

FIG. 8C is representative images of brain sections from the different groups stained for Aβ (red) and with DAPI (blue; scale bar, 2 mm).

FIGS. 8D-8K are quantification of average size of Aβ plaques (μm²) and coverage of Aβ (% of section) in the hippocampus (FIGS. 8D and 8E), cortex/striatum/amygdala (FIGS. 8F and 8G), thalamus/hypothalamus (FIGS. 8H and 8I) and in the whole brain section (FIGS. 8J and 8K). Results in FIGS. 8D-8K are presented as mean±s.e.m.; n=5 in mIgG2a group and n=6 in anti-Abeta antibody groups; One-way ANOVA with Bonferroni's multiple comparisons test.

FIGS. 9A-9G show pharmacological ablation of the dorsal meningeal lymphatic vessels in 5×FAD mice dampens Aβ plaque clearance by anti-Abeta antibody.

FIG. 9A is representative stereomicroscopy images of lymphatic vessels stained for LYVE-1 (green) around the confluence of sinuses (COS) and transverse sinus (TS) at the dorsal brain meninges or around the sigmoid (SS) and petrosquamosal (PSS) sinuses at the basal brain meninges still attached to the skull cap (in blue; scale bars, 500 μm).

FIGS. 9B-9E are graphs showing LYVE-1⁺ vessel total length (in mm) (FIG. 9B) and branching points (FIG. 9C) in the dorsal meninges and total length (in mm) (FIG. 9D) and branching points (FIG. 9E) in the basal meninges. The mice that were treated with Visudyne (results pooled from all the mice that were treated with ABETA Ab and mIgG2) have longer length (p value=0.0024, FIG. 9B) and more branching points (p value=0.0259, FIG. 9C) in the dorsal meninges than the mice that were treated with Visudyne and photoconversion (results pooled from all the mice that were treated with ABETA Ab and mIgG2). The mice that were treated with Visudyne and ABETA Ab have longer length (p value=0.0184, FIG. 9D) than the mice that were treated with Visudyne and photoconversion and ABETA Ab in the basal meninges. In comparisons between two groups, the mice that were treated with Visudyne and ABETA Ab have longer length (p value=0.0392, obtained by T test and not shown in FIG. 9B) and more branching points (p value=0.0596, obtained by T test and not shown in FIG. 9C) in the dorsal meninges than the mice that were treated with Visudyne and photoconversion and ABETA Ab. The p values in FIGS. 9B-9E result from the two-way ANOVA with multiple comparisons test unless otherwise indicated as a comparison between two groups using t test.

FIGS. 10A-10E show impairing meningeal lymphatic drainage affects the access of anti-Abeta antibody to Aβ plaques in the brain parenchyma. 5×FAD mice (5 months-old) with intact or ablated meningeal lymphatic vasculature were injected (i.c.m.) with 5 μL of anti-Abeta antibody (at 1 μg/L). One hour later, mice were transcardially perfused and the brain was collected for analysis.

FIG. 10A is images of ten different regions of the brain of 5×FAD mice from the Visudyne (Vis.) or Visudyne plus photoconversion (Vis./photo.) groups showing blood vessels stained for CD31 (blue), Aβ stained with Amylo-Glo RTD (green) and anti-Abeta antibody staining (red; scale bar, 200 μm).

FIGS. 10B and 10C are graphs with colocalization between CD31 and anti-Abeta antibody (% of CD31 signal occupied by anti-Abeta antibody) in each brain region (1 to 10) (FIG. 10B) or presented as the average of all regions (FIG. 10C).

FIGS. 10D and 10E are graphs with quantifications of colocalization between Aβ aggregates and anti-Abeta antibody (% of Aβ signal occupied by anti-Abeta antibody) in each brain region (1 to 10) (FIG. 10D) or presented as the average of all regions (FIG. 10E). Results in FIGS. 10B-10E are presented as mean±s.e.m.; n=5 per group; Two-way ANOVA with Sidak's multiple comparisons test in FIGS. 10B and 10D; two-tailed unpaired Student's T test in FIGS. 10C and 10E.

FIGS. 11A-11R show combination therapy with mVEGF-C and anti-Abeta antibody induces meningeal lymphangiogenesis and boosts brain Aβ plaque clearance.

FIG. 11A shows that adult 5×FAD mice were injected with 5 μL (i.c.m.) of AAV1 expressing enhanced green fluorescent protein (eGFP) or murine VEGF-C (mVEGF-C), under the cytomegalovirus (CMV) promoter (each at 10¹² GC/μL), in combination with either mIgG2a or anti-Abeta antibody (each at 1 μg/μL) as indicated in the scheme.

FIG. 11B is representative stereomicroscopy images of lymphatic vessels stained for LYVE-1 (green) around the transverse sinus (TS) at the dorsal brain meninges or around the sigmoid (SS) and petrosquamosal (PSS) sinuses at the basal brain meninges still attached to the skull cap (in blue; scale bars, 500 μm).

FIGS. 11C-11F are graphs showing LYVE-1⁺ vessel total length (in mm) (FIG. 11C) and branching points (FIG. 11D) in the dorsal meninges and total length (in mm) (FIG. 11E) and branching points (FIG. 11F) in the basal meninges. Results in FIGS. 11C-11F are presented as mean±s.e.m.; n=6 in mIgG2a groups and n=7 in anti-Abeta antibody groups; Two-way ANOVA with Sidak's multiple comparisons test; data in FIGS. 11B-11F is representative of 2 independent experiments.

FIG. 11C shows the differences in the total length in the dorsal meninges between mice that received different treatment. The comparisons between different groups and the corresponding p values are as follows: mIgG2 vs. ABETA Ab (results pooled from all the mice that were treated with eGFP or VEGF-c), p value=0.0005; mIgG2 and eGFP vs. ABETA Ab and VEGF-c, p value=0.0007; mIgG2 and VEGF-c vs. ABETA Ab and VEGF-c, p value=0.0025; ABETA Ab and eGFP vs. ABETA Ab and VEGF-c, p value=0.0233; eGFP vs. VEGF-c (results pooled from all the mice that were treated with mIgG2 or ABETA Ab), p value=0.0181.

FIG. 11D shows the differences in the number of branching point in the dorsal meninges between mice that received different treatments. The comparisons between different groups and the corresponding p values are as follows: mIgG2 vs. ABETA Ab (results pooled from all the mice that were treated with eGFP or VEGF-c), p value=0.0396; mIgG2 and eGFP vs. ABETA Ab and VEGF-c, p value=0.0244; ABETA Ab and eGFP vs. ABETA Ab and VEGF-c, p value=0.0406; eGFP vs. VEGF-c (results pooled from all the mice that were treated with mIgG2 or ABETA Ab), p value=0.0287.

FIG. 11G is representative images of brain sections from 5×FAD mice stained for Aβ (red) and with DAPI (blue; scale bar, 2 mm).

FIGS. 11H-11K are graphs showing the coverage of Aβ (% of brain region/section) in the hippocampus (FIG. 11H), cortex/striatum/amygdala (FIG. 11I), thalamus/hypothalamus (FIG. 11J) and the whole brain section (FIG. 11K).

FIG. 11H shows the differences in the coverage of Aβ in the hippocampus between mice that received different treatment. The comparisons between different groups and the corresponding p values are as follows: mIgG2 vs. ABETA Ab (results pooled from all the mice that were treated with eGFP or VEGF-c), p value=0.0011; mIgG2 and eGFP vs. ABETA Ab and VEGF-c, p value=0.0006; mIgG2 and VEGF-c vs. ABETA Ab and VEGF-c, p value=0.0450; eGFP vs. VEGF-c (results pooled from all the mice that were treated with mIgG2 or ABETA Ab), p value=0.0163.

FIG. 11I shows the differences in the coverage of Aβ in the cortex/striatum/amygdala between mice that received different treatment. The comparisons between different groups and the corresponding p values are as follows: mIgG2 vs. ABETA Ab (results pooled from all the mice that were treated with eGFP or VEGF-c), p value=0.0056; mIgG2 and eGFP vs. ABETA Ab and VEGF-c, p value=0.0062; eGFP vs. VEGF-c (results pooled from all the mice that were treated with mIgG2 or ABETA Ab), p value=0.0521.

FIG. 11J shows the differences in the coverage of Aβ in the thalamus and hypothalamus between mice that received different treatment. The comparisons between different groups and the corresponding p values are as follows: mIgG2 vs. ABETA Ab (results pooled from all the mice that were treated with eGFP or VEGF-c), p value=0.0092; mIgG2 and eGFP vs. ABETA Ab and VEGF-c, p value=0.0142.

FIG. 11K shows the differences in the coverage of Aβ in the whole between mice that received different treatment. The comparisons between different groups and the corresponding p values are as follows: mIgG2 vs. ABETA Ab (results pooled from all the mice that were treated with eGFP or VEGF-c), p value=0.0022; mIgG2 and eGFP vs. ABETA Ab and VEGF-c, p value=0.0142; eGFP vs. VEGF-c (results pooled from all the mice that were treated with mIgG2 or ABETA Ab), p value=0.0306.

FIG. 11L is representative images from the brain cortex stained for Aβ (blue), LAMP-1 (green) and Fibrinogen (red; scale bar, 200 μm).

FIGS. 11M and 11N are graphs showing the coverage (% of field) by LAMP-1⁺ dystrophic neurites (FIG. 11M) and Fibrinogen (FIG. 11N) in cortical vasculature.

FIG. 11M shows the differences in the coverage by LAMP-I dystropic neurites in cortical vasculature between mice that received different treatment. The comparisons between different groups and the corresponding p values are as follows: mIgG2 vs. ABETA Ab (results pooled from all the mice that were treated with eGFP or VEGF-c), p value=0.0001; mIgG2 and eGFP vs. ABETA Ab and VEGF-c, p value=0.0019; mIgG2 and VEGF-c vs. ABETA and VEGF-c, p value=0.0513; mIgG2 and eGFP vs. ABETA Ab and eGFP, p value=0.0545.

FIG. 11N shows the differences in the coverage by Fibrinogen in cortical vasculature between mice that received different treatment. The comparisons between different groups and the corresponding p values are as follows: eGFP vs. VEGF-c (results pooled from all the mice that were treated with mIgG2 or ABETA Ab), p value=0.0544.

FIG. 11O is representative images from the brain cortex stained for Aβ (blue), CD68 (green) and IBA1 (red; scale bar, 50 μm).

FIGS. 11P-11R are graphs showing the coverage by IBA1⁺ cells (in μm²) (FIG. 11P), number of peri-Aβ plaque IBA1⁺ cells (FIG. 11Q) and percentage (%) of IBA1 occupied by CD68 (FIG. 11R). Results in FIGS. 11H-11K, 11M, 11N, and 11P-11R are presented as mean s.e.m.; n=12 in mVEGF-C+mIgG2a and n=13 in eGFP+mIgG2a, eGFP+anti-Abeta antibody and mVEGF-C+anti-Abeta antibody; Two-way ANOVA with Sidak's multiple comparisons test, data in FIGS. 11G-11R results from 2 independent experiments.

FIG. 11Q shows the differences in the number of peri-Aβ plaque IBA1⁺ cells between mice that received different treatment. The comparisons between different groups and the corresponding p values are as follows: eGFP vs. VEGF-c (results pooled from all the mice that were treated with mIgG2 or ABETA Ab), p value=0.0139.

FIG. 11R shows the differences in the percentage (%) of IBA1 occupied by CD68 between mice that received different treatment. The comparisons between different groups and the corresponding p values are as follows: mIgG2 vs. ABETA Ab (pooled from all the mice that were treated with eGFP or VEGF-c), p value=0.0361; mIgG2 and eGFG vs. ABETA Ab and VEGF-c, p value=0.0328.

FIGS. 12A-12M show viral-mediated expression of mVEGF-C induces transcriptomic changes in aged meningeal LECs and improves the efficacy of anti-Abeta antibody treatment in aged AD transgenic mice.

FIG. 12A is representative images of meningeal whole mounts stained for CD31 (green) and LYVE-1 (red; scale bar, 1 mm; inset scale bar, 300 μm).

FIGS. 12B and 12C are graphs showing coverage of CD31⁺LYVE-1^{neg} vessels (% of meningeal whole mount) (FIG. 12B) and branching points and coverage of LYVE-1⁺ vessels (% of meningeal whole mount) (FIG. 12C). Results in FIGS. 12B and 12C are presented as mean±s.e.m.; n=7 in eGFP+mIgG2a and n=6 in eGFP+anti-Abeta antibody, mVEGF-C+mIgG2a and mVEGF-C+anti-Abeta antibody; Two-way ANOVA with Sidak's multiple comparisons test; data in FIGS. 12A-12C is representative of 2 independent experiments.

The left panel of FIG. 12C shows the differences in the coverage of branching points between mice that received different treatment. The comparisons between different groups and the corresponding p values are as follows: mIgG2 vs. ABETA Ab (pooled from all the mice that were treated with eGFP or VEGF-c), p value=0.0051; mIgG2 and eGFG vs. ABETA Ab and VEGF-c, p value=0.0329.

The right panel of FIG. 12C shows the differences in the coverage of LYVE-1⁺ vessels (% of meningeal whole mount) between mice that received different treatment. The comparisons between different groups and the corresponding p values are as follows: eGFP vs. VEGF-c (results pooled from all the mice that were treated with mIgG2 or ABETA Ab), p value=0.0540.

FIG. 12D shows that aged J20 mice (14-16 months-old) were injected with 5 μL (i.c.m.) of AAV1 expressing eGFP or mVEGF-C (each at 10¹² GC/L) in combination with either mIgG2a or anti-Abeta antibody (each at 1 μg/μL) as indicated in the scheme.

FIG. 12E is representative images of brain sections from J20 mice stained for Aβ (red) and with DAPI (blue; scale bar, 1 mm).

FIGS. 12F-12H are graphs showing coverage of Aβ (% of region) in the hippocampus (FIG. 12F), cortex/striatum/amygdala (FIG. 12G) and combined regions (FIG. 12H). Results in FIGS. 12F-12H are presented as mean±s.e.m.; n=8 in eGFP+anti-Abeta antibody and n=10 in mVEGF-C+anti-Abeta antibody; two-tailed unpaired Student's T test.

FIG. 12I shows aged APPswe mice (26-30 months-old) were injected with 5 μL (i.c.m.) of AAV1 expressing eGFP or mVEGF-C (each at 10¹² GC/L) in combination with either mIgG2a or anti-Abeta antibody (each at 1 μg/L) as indicated in the scheme.

FIG. 12J is representative images of brain sections from APPswe mice stained for Aβ (red) and with DAPI (blue; scale bar, 1 mm).

FIGS. 12K-12M are graphs showing coverage of Aβ (% of region) in the hippocampus (FIG. 12K), cortex/striatum/amygdala (FIG. 12L) and combined regions (FIG. 12M). Results in FIGS. 12K-12M are presented as mean±s.e.m.; n=11 per group; two-tailed unpaired Student's T test.

### DETAILED DESCRIPTION

This invention is based upon, at least partially, the unexpected discovery that the treatment with flow modulators, e.g., VEGF-c, in combination with a neurological therapeutic agent, e.g., an amyloid-β antibody, can synergize to reduce protein aggregates, e.g., amyloid-β plaques, in the central nervous system, e.g., brain. Flow modulators can increase flow for example, by increasing the diameter of a meningeal lymphatic vessel of the subject, by increasing the quantity of meningeal lymphatic vessels of the subject, and/or by increasing drainage through meningeal lymphatic vessels of the subject. Thus, fluid flow in the central nervous system of the subject can be increased. Neurological therapeutic agents interact with a target, e.g., protein aggregate, to reduce the contribution of the target to the pathogenesis of a neurologic disease. Without wishing to be bound by any theory, the flow modulators may facilitate the removal of the “end product” of the interaction between neurological therapeutic agents and the target, e.g., the complex formed between a pathological protein and an antibody, by various mechanisms as described in detail herein, e.g., increasing the drainage of the “end product” to certain specific sites, e.g., deep cervical lymph nodes, thereby improving the treatment of neurodegenerative disease. Alternatively, or in addition, the flow modulator may facilitate access of the neurological therapeutic agent to its target. Accordingly, a treatment combining a flow modulator described herein with a therapeutic agent described herein may improve the extent of the desired effect on the pathology of the neurological disease, e.g., reduction of pathological protein aggregates, reduction of inflammation at the site of aggregation, etc., as compared to a treatment with the therapeutic agent alone or as compared to a treatment with the flow modulator alone. Provided herein are compositions and methods using one or more flow modulators, e.g., VEGF-c, in combination with a neurological therapeutic agents to increase the therapeutic effect over the single agent alone.

As used herein, the term “reduce,” “reducing,” “reduction,” and the like, when it is used in the context of reducing a value or a range of a parameter, is defined as the reduction deduction in the value or range of the parameter as compared to a reference value or range. Similarly, the term “increase,” “increasing,” and the like, refers to the increase in the value or range of the parameter as compared to a reference value or range. The reference value or range is a baseline value or range that can be used to evaluate the change of a parameter. For example, a baseline value or range of a parameter of protein aggregate, such as average size of the protein aggregate, may refer to value or range of protein aggregate of a subject before receiving a treatment. In some embodiments, a baseline value or range may refer to the average value or range of a group of subjects who receive placebo treatment. In certain embodiments, a baseline value or range may refer to the average value or range of a population, such as a population of subjects that are diagnosed with Alzheimer's disease under certain criteria. For example, the baseline of amyloid beta plaque coverage may be the coverage of certain subject population diagnosed with Alzheimer's disease under certain criteria. Thus, a method reduce the amyloid beta plaque of a subject who is diagnosed with Alzheimer's disease under the same criteria when the average amyloid beta plaque coverage of the subject is lower than the average of population after the treatment. One of ordinary skill in the art can choose the baseline value or range of a parameter based on the treatment, diagnosis, or other criteria.

As used herein, the term “treat,” “treating,” “treatment,” and the like, when it is used in the context of treating a disease in a subject, is defined as the application or administration of a therapeutic agent to a patient, or application or administration of a therapeutic agent to an isolated tissue or cell line from a patient, said patient having a disease, a symptom of disease or a predisposition toward a disease, with the purpose to cure, heal, alleviate, relieve, alter, remedy, ameliorate, improve or affect the disease, the symptoms of disease or the predisposition toward disease. Thus, treating can include suppressing, inhibiting, preventing, treating, or a combination thereof. Treating refers, inter alia, to increasing time to disease progression, expediting remission, inducing remission, augmenting remission, speeding recovery, increasing efficacy of or decreasing resistance to alternative therapeutics, or a combination thereof “Suppressing” or “inhibiting”, refers, inter alia, to delaying the onset of symptoms, preventing relapse to a disease, decreasing the number or frequency of relapse episodes, increasing latency between symptomatic episodes, reducing the severity of symptoms, reducing the severity of an acute episode, reducing the number of symptoms, reducing the incidence of disease-related symptoms, reducing the latency of symptoms, ameliorating symptoms, reducing secondary symptoms, reducing secondary infections, prolonging patient survival, or a combination thereof. In one embodiment the symptoms are primary, while in another embodiment, symptoms are secondary. “Primary” refers to a symptom that is a direct result of a disorder, e.g., diabetes, while, secondary refers to a symptom that is derived from or consequent to a primary cause. Symptoms may be any manifestation of a disease or pathological condition.

By “treatment”, “prevention” or “amelioration” of a disease or disorder is meant delaying or preventing the onset of such a disease or disorder, reversing, alleviating, ameliorating, inhibiting, slowing down or stopping the progression, aggravation or deterioration the progression or severity of a condition associated with such a disease or disorder. In one embodiment, the symptoms of a disease or disorder are alleviated by at least 5%, at least 10%, at least 20%, at least 30%, at least 40%, or at least 50%. Accordingly, as used herein, the term “treatment” or “treating” includes any administration of a compound described herein and includes: (i) preventing the disease from occurring in a subject which may be predisposed to the disease but does not yet experience or display the pathology or symptomatology of the disease; (ii) inhibiting the disease in an subject that is experiencing or displaying the pathology or symptomatology of the diseased (i.e., arresting further development of the pathology and/or symptomatology); or (iii) ameliorating the disease in a subject that is experiencing or displaying the pathology or symptomatology of the diseased (i.e., reversing the pathology and/or symptomatology).

Efficacy of treatment is determined in association with any known method for diagnosing the disorder. Alleviation of one or more symptoms of the disorder indicates that the compound confers a clinical benefit. Any of the therapeutic methods described to above can be applied to any suitable subject including, for example, mammals such as dogs, cats, cows, horses, rabbits, monkeys, and most preferably, humans.

Traditionally, the central nervous system was viewed as being immune privileged, and was believed to lack a classical lymphatic drainage system. As described herein, a lymphatic system is present in meningeal spaces, and functions in draining macromolecules, immune cells, and debris from the central nervous system (CNS). Moreover, it has been discovered herein that combinations of agents can modulate drainage by the meningeal lymphatic drainage can affect certain diseases of the brain and central nervous system. In particular, as described in several embodiments herein, modulating lymphatic vessels to increase flow in accordance with some embodiments herein can synergize with neurological therapeutic agents to alleviate symptoms of neurological diseases, for example proteinopathies such as tauopathies and/or amyloidoses (e.g., AD), including cognitive symptoms, and accumulation of amyloid beta plaques. Accordingly, in some embodiments, methods, compositions, and uses for treating, preventing, inhibiting, or ameliorating symptoms of neurological diseases, for example proteinopathies such as tauopathies and/or amyloidoses (e.g., AD) are described. The neurological diseases can be associated with increased concentration and/or the accumulation of macromolecules, cells, and debris in the CNS (for example, AD, which is associated with the accumulation of amyloid beta plaques). The methods, compositions, and uses can increase drainage by lymphatic vessel, and thus increase flow in CSF and ISF. Several embodiments herein are particularly advantageous because they include one, several or all of the following benefits: (i) increased flow in the CNS; (ii) decreased accumulation of macromolecules, cells, or debris in the CNS (for example, decreased accumulation of amyloid beta); and (iii) maintenance of or improvement in motor and/or cognitive function (for example memory function) in a subject suffering from, suspected of having, and/or at risk for a neurological disease (such as dementia in a neurological disease such as AD).

It has been shown that meningeal lymphatic vessels mediate drainage in the CNS, and that impaired meningeal lymphatic function impacts brain homeostasis. See, e.g., PCT Pub. No. WO2017/210343 and US Patent Publication US20190269758A1, which is incorporated by reference herein in its entirety. Characteristics of meningeal lymphatic vessels are described for example, in PCT Pub. No. WO2017/210343 and US Patent Publication US20190269758A1 at Example 13. Immune cells such as T cells and dendritic cells accumulate in the meningeal lymphatic vessels (See, e.g., PCT Pub. No. WO2017/210343 and US Patent Publication US20190269758A1 at Examples 14-21). Impairing meningeal vessels significantly decreases drainage into deep cervical lymph nodes, and impact immune cell size and coverage, and inhibits immune cell migration (PCT Pub. No. WO2017/210343 and US Patent Publication US20190269758A1 at Examples 2 and 24-25).

Flow and Flow Modulators

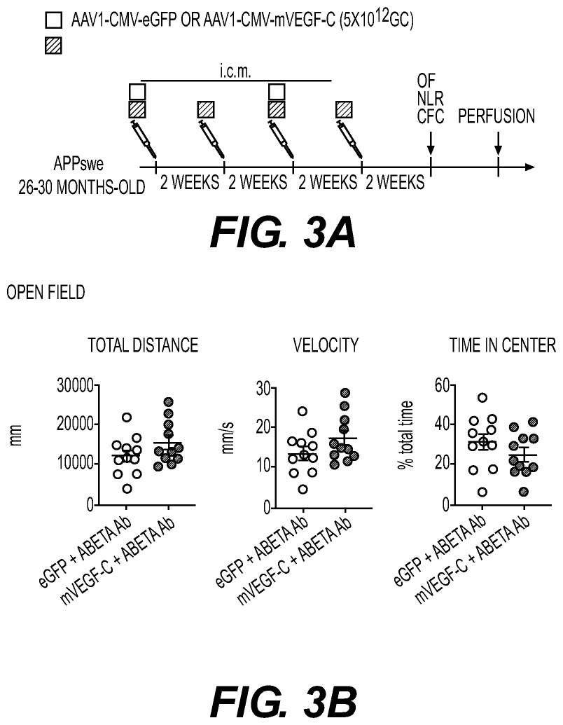

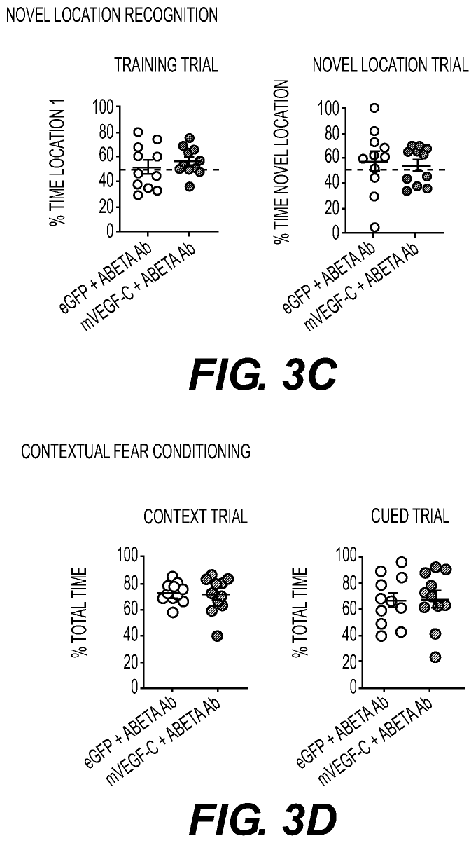

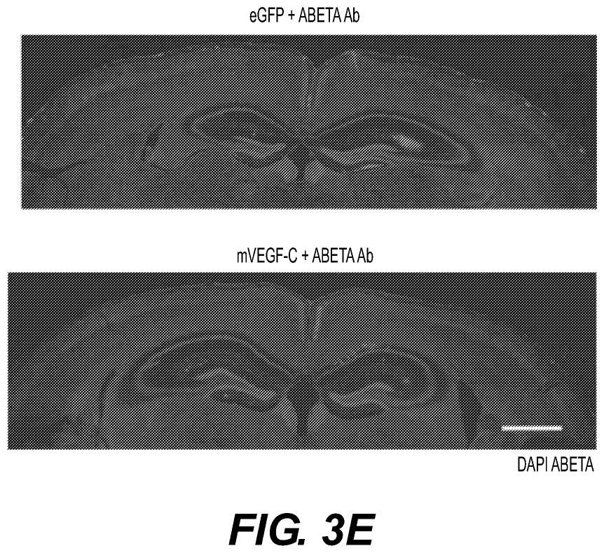

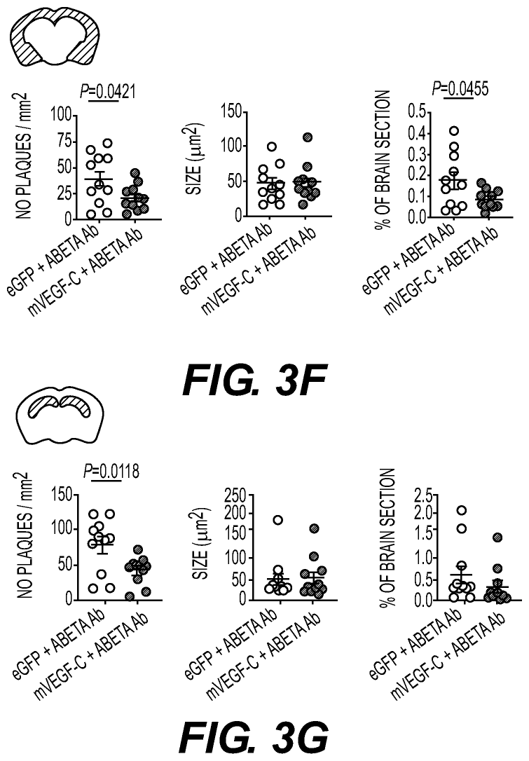

As used herein “flow” shall be given its ordinary meaning and shall also refer to a rate of perfusion through an area of the central nervous system of a subject. Flow in some embodiments, can be measured as a rate at which a label or tracer in CSF perfuses through a particular area of the central nervous system (see, e.g., FIGS. 3A-3J of WO2017/210343 and US Patent Publication US20190269758A1). As such, flow can be compared between two subjects or two sets of conditions by ascertaining how quickly an injected label or tracer perfuses throughout a particular area or volume of the brain and/or other portion of the CNS.

As used herein, “flow modulators” shall be given its ordinary meaning and shall also broadly refer to classes of compositions that can increase or decrease the passage of substances into and out of meningeal lymphatic vessels, and thus can modulate flow in CSF and ISF, and/or, can modulate immune cell migration within, into, and out of the meningeal lymphatic vessels. In one example, the lymphatic flow modulator modulates the status of lymphatic endothelial cells, i.e., is a lymphatic endothelial cell modulator.

It was shown that, increasing the passage and substances into and out of meningeal lymphatic vessels can increase flow in CSF and ISF (see Examples 4-6 and FIGS. 26-29 of WO 2017/210343 and US Patent Publication US20190269758A1). Without being limited by theory, it is contemplated, according to several embodiments herein, that removal of macromolecules through meningeal lymphatic vessels can keep their concentrations low in the CSF, allowing a gradient to clear macromolecules from the parenchyma. As such, the higher the rate of drainage of molecules by meningeal lymphatic vessels, the higher the rate of flow of molecules in the CNS (e.g., in CSF and ISF). Furthermore, the higher the rate of fluid flow and drainage in the CNS, the higher the rate of clearance and/or the lower the concentration of cells, macromolecules, waste, and debris form the CNS. In some embodiments, flow modulators increase the diameter of meningeal lymphatic vessels, which increases drainage, resulting in increased flow in the CSF and ISF. In some embodiments, flow modulators increase the number of meningeal lymphatic vessels, thus increasing net drainage, resulting in increased flow in the CSF and ISF. Examples of suitable flow modulators for increasing flow (for example by increasing meningeal lymphatic vessel diameter) in accordance with various embodiments herein include, but are not limited to, VEGFR3 agonists, for example VEGF-c and VEGF-d, and Fibroblast Growth Factor 2 (FGF2), and functional fragments, variants, analogs, and mimetics of these molecules.

In some embodiments, the flow modulators are proteins or peptides or the polynucleotides encoding the proteins or peptides. In certain embodiments, the flow modulator comprises lymphangiogenic growth factors or the polynucleotides encoding the lymphangiogenic growth factors. Lymphangiogenic growth factors are peptide growth factors capable of inducing the growth of new lymphatic vessels in vivo in lymphangiogenesis and/or increasing the diameter of the existing lymphatic vessels. Exemplary lymphangiogenic growth factors include, but are not limited to VEGFR3 agonist, e.g., VEGF-c, VEGF-d, FGF2, or a variant thereof. In one embodiment, the flow modulator is a lymphatic endothelial cell modulator.

The term “variant,” as used herein, refers to a polypeptide that is derived by incorporation of one or more amino acid insertions, substitutions, or deletions in a precursor polypeptide or polynucleotide (e.g., “parent” polypeptide or polynucleotide). In certain embodiments, a variant polypeptide or polynucleotide has at least about 85% amino acid or nucleotide sequence identity, e.g., about 90%, about 91%, about 92%, about 93%, about 94%, about 95%, about 96%, about 97%, about 98%, about 99%, or about 100%, amino acid or nucleotide sequence identity to the entire amino acid or nucleotide sequence of a parent polypeptide or polynucleotide. A variant of a protein or peptide maintains the functions or activities of the protein. For example, a variant of a lymphangiogenic growth factor maintains the function or activities of inducing the growth of new lymphatic vessels and/or increasing the diameter of existing lymphatic vessels. In the case of a polynucleotide, a variant thereof maintain its function or activities of the parent polynucleotide. For example, a variant polynucleotide may encode a protein or peptide that has similar functions or activities of the polypeptide encoded by the parent polynucleotide. The term “sequence identity,” as used herein, refers to a comparison between pairs of nucleic acid or amino acid molecules, i.e., the relatedness between two amino acid sequences or between two nucleotide sequences. In general, the sequences are aligned so that the highest order match is obtained. Methods for determining sequence identity are known and can be determined by commercially available computer programs that can calculate the percentage of identity between two or more sequences. A typical example of such a computer program is CLUSTAL.

In certain embodiments, the flow modulator comprises a nucleic acid or polynucleotide. The nucleic acids or polynucleotides of the invention may include deoxynucleotides, ribonucleotides, modified deoxynucleotides, modified ribonucleotides (e.g., chemical modifications, such as modifications that alter the backbone linkages, sugar molecules, and/or nucleic acid bases), and artificial nucleic acids. In some embodiments, the polynucleotide includes, but is not limited to, genomic DNA, cDNA, oligonucleotides, peptide nucleic acids (PNA) or peptide oligonucleotide conjugates, locked nucleic acids (LNA), bridged nucleic acids (BNA), polyamides, triplex forming oligonucleotides, modified DNA, antisense DNA oligonucleotides, tRNA, mPvNA, rPvNA, modified RNA, miRNA, gRNA, and siRNA or other RNA or DNA molecules.

In certain embodiments, a polynucleotide comprises a sequence that encodes a peptide or a protein, e.g., the lymphangiogenic growth factors, disclosed herein. The polynucleotide may be comprised in an expression vectors described herein. The polynucleotide may be an mRNA encoding the lymphangiogenic growth factor as described herein.

In methods, uses, or compositions of some embodiments, a flow modulator (e.g., VEGFR3 agonists, or FGF2) comprises or consists essentially of a polypeptide or protein that comprises a modification, for example a glycosylation, PEGylation, or the like.

In some embodiments, a composition or composition for use in accordance with methods and uses described herein comprises or consists essentially of one or more flow modulators (e.g., VEGFR3 agonists, or FGF2), one or more neurological therapeutic agents (e.g., amyloid beta antibody), and a pharmaceutically acceptable diluent or carrier. Examples of suitable pharmaceutically acceptable carriers and formulations are described in “Remington: The Science and Practice of Pharmacy” 22nd Revised Edition, Pharmaceutical Press, Philadelphia, 2012, which is hereby incorporated by reference in its entirety. In some embodiments, the composition comprises or consists essentially of a unit dose of a flow modulator effective for increasing flow of CNS fluids, increasing clearance of molecules in the CNS, or reducing an aggregate of a protein or peptide, e.g., amyloid beta, in the central nervous system. As used herein, reducing an aggregate of a protein or peptide includes the reduction of the number of the aggregates, reduction in the average area of the aggregates, reduction in the total coverage of the aggregates (the ratio of the area covered by the aggregates to the total area of a selected tissue area, such as a cross section of brain). In some embodiments, the composition comprises, or consists essentially of a single unit dose of flow modulator effective for increasing flow, increasing clearance reducing accumulated amyloid beta plaques, reducing immune cell migration, or reducing inflammation. In some embodiments, the effective amount of flow modulator is about 0.00015 mg/kg to about 1.5 mg/kg (including any other amount or range contemplated as a therapeutically effective amount of a compound as disclosed herein), is less than about 1.5 mg/kg (including any other range contemplated as a therapeutically effective amount of a compound as disclosed herein), or is greater than 0.00015 mg/kg (including any other range contemplated as a therapeutically effective amount of a compound as disclosed herein).

VEGFR3 Agonists

VEGFR3, also known as FLT4, is a receptor tyrosine kinase, and its signaling pathway has been implicated in embryonic vascular development, and adult lymphangiogenesis. Upon binding of ligand, VEGFR3 dimerizes, and is activated through autophosphorylation. It is shown herein that VEGFR3 agonists are a class of flow modulators that increase the diameter of meningeal lymphatic vessels, and which increase drainage and the flow of CSF and ISF in accordance with some embodiments herein (see Examples 4-6, FIGS. 26, 27A-27D, 28A, and 28C of WO2017/210343 and US Patent Publication US20190269758A1). As such, VEGFR3 agonists are suitable for methods, compositions, and uses for treating, ameliorating, reducing the symptoms of, or preventing neurological diseases associated with accumulation of molecules in the brain, for example proteinopathies as described herein (e.g., tauopathies and/or amyloidoses such as AD), in accordance with some embodiments herein. Accordingly, in some embodiments, such as methods or compositions for which increased drainage and flow are desired, a flow modulator comprises, consists of, or consists essentially of a VEGFR3 agonist. In certain embodiments, the VEGFR3 agonist is a protein or polypeptide.

An effective amount of VEGFR3 agonist in accordance with methods, compositions, and uses herein can be understood in terms of its ability to increase meningeal vessel diameter, by its ability to increase flow of CSF or ISF, or by its ability to treat, ameliorate, or prevent (by its ability to increase clearance of substances such as proteins from the CNS, for example amyloid beta), symptoms of a neurological disease such as proteinopathies as described herein (e.g., tauopathies and/or amyloidoses such as AD), for example quantities of beta-amyloid plaques or measurements of cognitive function. Accordingly, in compositions, methods, and uses of some embodiments, an effective amount of VEGFR3 agonist increases meningeal vessel diameter by at least about 2%, for example, at least about 2%, 3%, 4%, 5%, 6%, 7%, 8%, 9%, 10%, 15%, 20%, 25%, 30%, 35%, 40%, 45%, 50%, 55%, 60%, 65%, 70%, or 75%, including ranges between any two of the listed values. In compositions, methods, and uses of some embodiments, an effective amount of VEGFR3 agonist increases flow of the CSF or ISF by at least about 2%, for example, at least about 2%, 3%, 4%, 5%, 6%, 7%, 8%, 9%, 10%, 15%, 20%, 25%, 30%, 35%, 40%, 45%, 50%, 55%, 60%, 65%, 70%, or 75%, including ranges between any two of the listed values.

Example VEGFR3 agonists suitable for methods, uses, and compositions in accordance with some embodiments herein include the polypeptides VEGF-c and VEGF-d, the amino acid sequences of which are shown in Table 1, below, as well as variants and analogs of VEGF-c and/or VEGF-d. By way of example, VEGF-c, in accordance with some embodiments herein has been demonstrated to increase the diameters of meningeal lymphatic vessels, and to increase drainage, CSF and ISF flow, and clearance in the CNS. See Example 4 of WO2017/210343 and US Patent Publication US20190269758A1. In some embodiments, a VEGFR3 agonist comprises, consists of, or consists essentially of VEGF-c. In some embodiments, a VEGFR3 agonist comprises, consists of, or consists essentially of VEGF-d. In some embodiments, VEGF-c and VEGF-d together agonize VEGFR3, and can be provided in a single composition, or in separate compositions. In some embodiments, a VEGFR3 agonist comprises, consists or, or consists essentially of an analog, variant, or functional fragment, such as a mutant, ortholog, fragment, or truncation of VEGF-c or VEGF-d, for example a polypeptide comprising, or consisting essentially of an amino acid sequence having at least about 80% identity to SEQ ID NO: 1 or 2 or 3, for example at least about 80%, 85%, 90%, 95%, 97%, 98%, or 99% identity, including ranges between any two of the listed values.

As shown in Examples 5, 6, and 11 of WO2017/210343 and US Patent Publication US20190269758A1, exogenous nucleotides encoding a VEGFR3 agonist, such as VEGF-c, can also be suitable for methods, uses, and compositions in accordance with some embodiments herein. Accordingly, in some embodiments, a nucleotide encoding VEGF-c or VEGF-d as describe herein is expressed in a subject in order to administer the VEGFR3 agonist to a subject. For example, an exogenous vector such as a retroviral, lentiviral, adenoviral, or adeno-associated viral vector comprising or consisting essentially of a nucleic acid encoding a VEGFR agonist as described here can be inserted into a host nucleic acid of the subject (for example in the genome of a somatic cell of the subject). In some embodiments, the vector further comprises transcriptional machinery to facilitate the transcription of the nucleic acid encoding the VEGFR agonist, for example, a core promoter, transcriptional enhancer elements, insulator elements (to insulate from repressive chromatin environments), and the like.

TABLE 1
Example VEGFR3 agonists

| | UniProt | |
|---|---|---|
| Agonist | Accession | SEQ ID NO: |
| VEGF-c | P49769 | 1(MHLLGFFSVACSLLAAALLPGPREAPAAAAAFESGLDLSDAEP |
| | | DAGEATAYASKDLEEQLRSVSSVDELMTVLYPEYWKMYKCQLRKG |
| | | GWQHNREQANLNSRTEETIKFAAAHYNTEILKSIDNEWRKTQCMP |
| | | REVCIDVGKEFGVATNTFFKPPCVSVYRCGGCCNSEGLQCMNTST |
| | | SYLSKTLFEITVPLSQGPKPVTISFANHTSCRCMSKLDVYRQVHS |
| | | IIRRSLPATLPQCQAANKTCPTNYMWNNHICRCLAQEDEMESSDA |
| | | GDDSTDGFHDICGPNKELDEETCQCVCRAGLRPASCGPHKELDRN |
| | | SCQCVCKNKLFPSQCGANREFDENTCQCVCKRTCPRNQPLNPGKC |
| | | ACECTESPQKCLLKGKKFHHQTCSCYRRPCTNRQKACEPGESYSE |
| | | EVCRCVPSYWKRPQMS) |
| VEGF-d | O43915 | 2 |
| | | (MYREWVVVNVFMMLYVQLVQGSSNEHGPVKRSSQSTLERSEQQI |
| | | RAASSLEELLRITHSEDWKLWRCRLRLKSFTSMDSRSASHRSTRE |
| | | AATFYDIETLKVIDEEWQRTQCSPRETCVEVASELGKSTNTFFKP |
| | | PCVNVFRCGGCCNEESLICMNTSTSYISKQLFEISVPLTSVPELV |
| | | PVKVANHTGCKCLPTAPRHPYSIIRRSIQIPEEDRCSHSKKLCPI |
| | | DMLWDSNKCKCVLQEENPLAGTEDHSHLQEPALCGPHMMEDEDRC |
| | | ECVCKTPCPKDLIQHPKNCSCFECKESLETCCQKHKLFHPDTCSC |
| | | EDRCPFHTRPCASGKTACAKHCRFPKEKRAAQGPHSRKNP) |
| VEGF-C156S | Q6FH59 | 3(MHLLGFFSVACSLLAAALLPGPREAPAAAAAFESGLDLSDAEP |
| | | DAGEATAYASKDLEEQLRSVSSVDELMTVLYPEYWKMYKCQLRKG |
| | | GWQHNREQANLNSRTEETIKFAAAHYNTEILKSIDNEWRKTQCMP |
| | | REVCIDVGKEFGVATNTFFKPPCVSVYRCGGCCNSEGLQCMNTST |
| | | SYLSKTLFEITVPLSQGPKPVTISFANHTSCRCMSKLDVYRQVHS |
| | | IIRRSLPATLPQCQAANKTCPTNYMWNNHICRCLAQEDEMESSDA |
| | | GDDSTDGFHDICGPNKELDEETCQCVCRAGLRPASCGPHKELDRN |
| | | SCQCVCKNKLFPSQCGANREFDENTCQCVCKRTCPRNQPLNPGKC |
| | | AYECTESPQKCLLKGKKFHHQTCSCYRRPCTNRQKACEPGESYSE |
| | | EVCRCVPSYWKRPQMS) |

In methods or compositions of some embodiments, the VEGFR3 agonist comprises a modification, for example a glycosylation, PEGylation, or the like. In some embodiments, a composition for use in accordance with the methods described herein comprises the VEGFR3 agonist (e.g., VEGF-c and/or VEGF-d), and a pharmaceutically acceptable diluent or carrier.

FGF2

In some embodiments, the flow modulator comprises or consists essentially of Fibroblast Growth Factor 2 (FGF2), or a variant thereof. Without being limited by theory, it is contemplated that FGF2 can increase drainage (and flow) of CSF or ISF in meningeal lymphatic vessel, for example by increasing the diameter of meningeal lymphatic vessel. An example of a suitable FGF2 amino acid sequence in accordance with some embodiments is provided as Uniprot Accession No. P09038 (human FGF2) (SEQ ID NO: 4—

MVGVGGGDVEDVTPRPGGCQISGRGARGCNGIPGAAAWEAALPRRRPRR
HPSVNPRSRAAGSPRTRGRRTEERPSGSRLGDRGRGRALPGGRLGGRGR
GRAPERVGGRGRGRGTAAPRAAPAARGSRPGPAGTMAAGSITTLPALPE
DGGSGAFPPGHFKDPKRLYCKNGGFFLRIHPDGRVDGVREKSDPHIKLQ
LQAEERGVVSIKGVCANRYLAMKEDGRLLASKCVTDECFFFERLESNNY
NTYRSRKYTSWYVALKRTGQYKLGSKTGPGQKAILFLPMSAKS).

Routes of Administration

Flow modulators (e.g., FGF2 or VEGFR3 agonists such as VEGF-c) and/or neurological therapeutic agents (e.g., amyloid beta antibodies) in accordance with methods, compositions for use, or uses of embodiments herein can be administered to a subject using any of a number of suitable routes of administration, provided that the route of administration administers the flow modulator to the CNS (such as the meningeal space) of a subject. It is noted that many compounds do not readily cross the blood-brain barrier, and as such, some routes of administration such as intravenous will not necessarily deliver the flow modulator and/or neurological therapeutic agent to the CNS (unless the flow modulator can readily cross the blood-brain barrier). By “administering to the CNS of a subject,” as used herein (including variations of this root term), it is not necessarily required that a flow modulator and/or neurological therapeutic agent be administered directly to the CNS (such as meningeal space), but rather, this term encompasses administering a flow modulator and/or neurological therapeutic agent directly and/or indirectly to the CNS. It is contemplated that administering the flow modulator and/or neurological therapeutic agent so that it is in fluid communication with the CNS (e.g., meningeal space) of the subject in accordance with some embodiments herein (typically by administering the flow modulator and/or neurological therapeutic agent on the “brain” side of the blood-brain barrier), the flow modulator and/or neurological therapeutic agent will be administered to the meningeal space. Accordingly, in some embodiments, the flow modulator and/or neurological therapeutic agent is not administered systemically. In some embodiments, the flow modulator and/or neurological therapeutic agent is not administered systemically, but rather is administered to a fluid, tissue, or organ in fluid communication with the CNS (such as the meningeal space), and on the brain side of the blood-brain barrier. In some embodiments, the flow modulator and/or neurological therapeutic agent is not administered systemically, but rather is administered to the CNS. In some embodiments, the flow modulator and/or neurological therapeutic agent is administered to the CNS, but is not administered to any organ or tissue outside of the CNS. In some embodiments, the flow modulator and/or neurological therapeutic agent is not administered to the blood. In some embodiments, the flow modulator and/or neurological therapeutic agent is not administered to a tumor, or to the vasculature of a tumor. In some embodiments, the flow modulator and/or neurological therapeutic agent is administered systemically. It is contemplated that a flow modulator and neurological therapeutic agent can be administered together (in a single composition), or separately (e.g., in separate compositions, which can be administered to the same location at the same or different times, or can be administered to different locations at the same or different times). Accordingly, in some embodiments, a flow modulator and neurological therapeutic agent can be administered together (in a single composition). In some embodiments, a flow modulator and neurological therapeutic agent are administered in separate compositions. In some embodiments, a flow modulator and neurological therapeutic agent are administered in separate compositions to different sites of administration on a subject at the same time. In some embodiments, a flow modulator and neurological therapeutic agent are administered in separate compositions to different sites of administration on a subject at different times (for example, the flow modulator can be administered prior to the neurological therapeutic agent, or the flow modulator can be administered after the neurological therapeutic agent). In some embodiments, a flow modulator and neurological therapeutic agent are administered in separate compositions to the same site of administration on a subject at different times (for example, the flow modulator can be administered prior to the neurological therapeutic agent, or the flow modulator can be administered after the neurological therapeutic agent).

The flow modulator and/or neurologic therapeutic agent may be in a “biologically compatible form suitable for administration in vivo,” which is a form of the flow modulator and/or neurological therapeutic agent to be administered in which any toxic effects are outweighed by the therapeutic effects of the flow modulator and/or neurological therapeutic agent. The term “subject” is intended to include living organisms in which a neurological disease or disorder can be identified, e.g., mammals. Examples of subjects include humans, dogs, cats, mice, rats, and transgenic species thereof.

Administration of a flow modulator (e.g., FGF2 or VEGFR3 agonist such as VEGF-c) and/or neurological therapeutic agent (e.g., amyloid beta antibody) as described herein can be in any pharmacological form including a therapeutically active amount of an agent alone or in combination with a pharmaceutically acceptable carrier.

Administration of a therapeutically active amount of the therapeutic composition (e.g., flow modulator and/or neurological therapeutic agent) of the present disclosure is defined as an amount effective, at dosages and for periods of time necessary, to achieve the desired result. For example, a therapeutically active amount of a flow modulator and/or neurological therapeutic agent may vary according to factors such as the disease state, age, sex, and weight of the individual, and the ability of peptide to elicit a desired response in the individual. Dosage regimens can be adjusted to provide the optimum therapeutic response. For example, several divided doses can be administered daily or the dose can be proportionally reduced as indicated by the exigencies of the therapeutic situation. The flow modulator (e.g., FGF2 or VEGFR3 agonist such as VEGF-c) and/or neurological therapeutic agent (e.g., amyloid beta antibody) of the disclosure described herein can be administered in a convenient manner such as by transcranial administration, intrathecal administration, intraventricular administration, and/or intraparenchymal administration by contact with cerebral spinal fluid (CSF) of the subject, administration by pumping into CSF of the subject, administration by implantation into the skull or brain, administration by contacting a thinned skull or skull portion of the subject with the agent, injection (subcutaneous, intravenous, etc.), oral administration, inhalation, transdermal application, or rectal administration. In some embodiments, the flow modulator and/or neurological therapeutic agent are administered to the subject (or formulated for administration) by a route selected from the group consisting of intrathecal administration, intraventricular administration, intraparenchymal administration, nasal administration, transcranial administration, contact with cerebral spinal fluid (CSF) of the subject, pumping into CSF of the subject, implantation into the skull or brain, contacting a thinned skull or skull portion of the subject with the neurological therapeutic agent, expression in the subject of a nucleic acid encoding the neurological therapeutic agent, intravenous infusion, or a combination of any of the listed routes. For example, the flow modulator and/or neurological therapeutic agent can be administered to the subject (or formulated for administration) by a route selected from the group consisting of: intrathecal administration, intraventricular administration, intraparenchymal administration, nasal administration, transcranial administration, contact with cerebral spinal fluid (CSF) of the subject, pumping into CSF of the subject, implantation into the skull or brain, contacting a thinned skull or skull portion of the subject with the neurological therapeutic agent, expression in the subject of a nucleic acid encoding the neurological therapeutic agent, or a combination of any of the listed routes. The flow modulator and the neurological therapeutic agent may be administered by the same route, or by different routes. In some embodiments, the neurological therapeutic agent is administered by intravenous infusion, and the flow modulator is administered by any route of administration described herein. In some embodiments, the neurological therapeutic agent and the flow modulator are both administered by intravenous infusion. Depending on the route of administration, the flow modulator and/or neurological therapeutic agent can be coated in a material to protect the compound from the action of enzymes, acids and other natural conditions which may inactivate the compound. For example, for administration of flow modulator and/or neurological therapeutic agent, by other than parenteral administration, it may be desirable to coat the flow modulator and/or neurological therapeutic agent with, or co-administer the flow modulator and/or neurological therapeutic agent with, a material to prevent its inactivation. For example, the neurological therapeutic agent alone, or with the flow modulator can be administered via intravenous infusion. The intravenous infusion may be repeated, for example, once every 1 week, 2 weeks, 3 weeks, 4 weeks, month, or two months including ranges between any two of the listed values, for example, once every 1-2 weeks, 1-4 weeks, 1 week-1 month, 2-4 weeks, or 2 weeks-1 month. By way of example, the neurological therapeutic agent may be a monoclonal antibody specific for amyloid beta, for example bapineuzumab, gantenerumab, aducanumab, solanezumab, and/or crenezumab. In some embodiments, the monoclonal antibody specific for amyloid beta, such as bapineuzumab, gantenerumab, aducanumab, solanezumab, and/or crenezumab, is administered monthly via intravenous infusion.

In some embodiments, the flow modulator (e.g., FGF2 or VEGFR3 agonist such as VEGF-c) and/or neurological therapeutic agent (e.g., amyloid beta antibody) is administered nasally. For example, the flow modulator and/or neurological therapeutic agent can be provided in a nasal spray, or can be contacted directly with a nasal mucous membrane.

In some embodiments, the flow modulator (e.g., FGF2 or VEGFR3 agonist such as VEGF-c) and/or neurological therapeutic agent (e.g., amyloid beta antibody) is administered through contacting with CSF of the subject. For example, the flow modulator and/or neurological therapeutic agent can be directly injected into CSF of a patient (for example into a ventricle of the brain). Suitable apparatuses for injection can include a syringe, or a pump that is inserted or implanted in the subject and in fluid communication with CSF. In some embodiments, a composition comprising or consisting essentially of the flow modulator and/or neurological therapeutic agent, for example a slow-release gel, is implanted in a subject so that it is in fluid communication with CSF of the subject, and thus contacts the CSF.

In some embodiments, the flow modulator (e.g., FGF2 or VEGFR3 agonist such as VEGF-c) and/or neurological therapeutic agent (amyloid beta antibody) is administered transcranially. For example, a composition comprising or consisting essentially of the flow modulator and/or neurological therapeutic agent such as a gel can be placed on an outer portion of the subject's skull, and can pass through the subject's skull. In some embodiments, the flow modulator and/or neurological therapeutic agent is contacted with a thinned portion of the subject's skull to facilitate transcranial delivery.

In some embodiments, the flow modulator (e.g., FGF2 or VEGFR3 agonist such as VEGF-c) and/or neurological therapeutic agent (e.g., amyloid beta antibody) is administered by expressing a nucleic acid encoding the flow modulator and/or neurological therapeutic agent in the subject. A vector comprising or consisting essentially of the nucleic acid, for example a viral vector such as a retroviral vector, lentiviral vector, or adenoviral vector, or adeno-associated viral vector (AAV) can be administered to a subject as described herein, for example via injection or inhalation. In some embodiments, the nucleic acid is administered as an mRNA as described herein, for example as a chemically modified messenger RNA (mRNA). In some embodiments, expression of the nucleic acid is induced in the subject, for example via a drug or optical regulator of transcription.

In some embodiments, the flow modulator (e.g. the VEGFR3 antagonist such as VEGF-c or FGF2) and/or neurological therapeutic agent (e.g., amyloid beta antibody) is administered selectively to the meningeal space of the subject, or is for use in administration selectively to the meningeal space of the subject. As used herein administered “selectively” and variations of the root term indicate that the flow modulator is administered preferentially to the indicated target (e.g. meningeal space) compared to other tissues or organs on the same side of the blood brain barrier. As such, direct injection to meningeal spaces of the brain would represent “selective” administration, whereas administration to CSF in general via a spinal injection would not. In some embodiments, the flow modulator and/or neurological therapeutic agent is administered selectively to the meningeal space, and not to portions of the CNS outside of the meningeal space, nor to any tissues or organs outside of the CNS. In some embodiments, the flow modulator is administered selectively to the CNS, and not to tissue or organs outside of the CNS such as the peripheral nervous system, muscles, the gastrointestinal system, musculature, or vasculature.

For any of the routes of administration listed herein in accordance with methods, uses, and compositions herein, it is contemplated that a flow modulator (e.g., FGF2 or VEGFR3 agonist such as VEGF-c) and/or neurological therapeutic agent (e.g., amyloid beta antibody) can be administered in a single administration, or in two or more administrations, which can be separated by a period of time. For example, in some embodiments, the flow modulator and/or neurological therapeutic agent as described herein can be administered via a route of administration as described herein hourly, daily, every other day, every three days, every four days, every five days, every six days, weekly, biweekly, monthly, bimonthly, and the like. In some embodiments, the flow modulator and/or neurological therapeutic agent is administered in a single administration, but not in any additional administrations.

Some embodiments include methods of making a composition or medicament comprising or consisting essentially of a flow modulator (e.g., FGF2 or VEGFR3 agonist such as VEGF-c) and/or neurological therapeutic agent (e.g., amyloid beta antibody) as described herein suitable for administration according to a route of administration as described herein. For example, in some embodiments, a composition comprising or consisting essentially of a VEGFR3 agonist (such as VEGF-c) and an amyloid beta antibody is prepared for nasal administration, administration to the CSF, or transcranial administration. For example, in some embodiments, a composition comprising or consisting essentially of a VEGFR3 antagonist (such as VEGF-c) and amyloid beta antibody is prepared for nasal administration, administration by contacting with CSF, or transcranial administration.

Conventional methods, known to those of ordinary skill in the art of medicine, can be used to administer a pharmaceutical composition comprising a flow modulator (e.g., the VEGFR3 antagonist or FGF2) and/or neurological therapeutic agent (e.g., amyloid beta antibody) to the subject, depending upon the type of disease to be treated or the site of the disease. This composition can also be administered via other conventional routes, e.g., administered orally, parenterally, by inhalation spray (e.g., aerosol), topically, rectally, nasally, buccally, sublingually, vaginally, via local injection, or via an implanted reservoir. The term “parenteral” as used herein includes subcutaneous, intracutaneous, intravenous, intramuscular, intranasal, intraocular, intratracheal, intraarticular, intraarterial, intrasynovial, intrasternal, intrathecal, intraventricular, intraparenchymal, intralesional, intracranial injection or infusion techniques, transdermal. The compositions may be administered orally, intraperitoneally or intravenously. In some embodiments, the administration route is oral. In some embodiments, the administration is via injection. In some embodiments, the administration is via local injection. In some embodiments, the administration of the compound is into the cerebrospinal fluid (CSF) of said subject. In some embodiments, the administration of the compound is via intracerebroventricular injection. In some embodiments, the administration is transdermal, e.g., via application of an ointment containing the therapeutic to the head (scalp skin) of said subject.

In addition, it can be administered to the subject via injectable depot routes of administration such as using 1-, 3-, or 6-month depot injectable or biodegradable materials and methods. In some examples, the pharmaceutical composition is administered intraocularly or intravitreally.

In some examples, the pharmaceutical composition comprising the flow modulator (e.g. the VEGFR3 antagonist or FGF2) and/or neurological therapeutic agent (e.g., amyloid beta antibody) is administered intrathecally, intraventricularly, and/or intraparenchymally, e.g., via an injection into the spinal canal, or into the subarachnoid space.

In some embodiments, the flow modulator (e.g., FGF2 or VEGFR3 agonist such as VEGF-c) and/or neurological therapeutic agent (e.g., amyloid beta antibody) is administered intraventricularly, i.e., into the lateral ventricle of the brain. In some embodiments, the flow modulator and/or neurological therapeutic agent is administered intraparenchymally, i.e., into the brain parenchyma. In some embodiments, the flow modulator and/or neurological therapeutic agent is administered and delivered through an implanted catheter connected to a pump, which contains a reservoir of the composition and controls the rate of delivery. In some embodiments, the flow modulator and/or neurological therapeutic agent is released into the cerebrospinal fluid (CSF) of the cisterna magna. In any of these embodiments, the catheter can either be introduced between the first and second cervical vertebrae (C1-C2 interspace) or into the intracranial ventricles. In some embodiments, an Ommaya reservoir (consisting of a catheter in one lateral ventricle attached to a reservoir implanted under the scalp) is used as an intraventricular catheter system for the administration and delivery of the flow modulator and/or neurological therapeutic agent into the cerebrospinal fluid.

In some embodiments, the flow modulator (e.g., FGF2 or VEGFR3 agonist such as VEGF-c) and/or neurological therapeutic agent (e.g., amyloid beta antibody) is administered at the site of an amyloid beta plaque.

In one embodiment, a flow modulator (e.g., FGF2 or VEGFR3 agonist such as VEGF-c) and/or neurological therapeutic agent (e.g., amyloid beta antibody) is administered via site-specific or targeted local delivery techniques. Examples of site-specific or targeted local delivery techniques include various implantable depot sources of the flow modulator or local delivery catheters, such as infusion catheters, an indwelling catheter, or a needle catheter, synthetic grafts, adventitial wraps, shunts and stents or other implantable devices, site specific carriers, direct injection, or direct application. See, e.g., PCT Publication No. WO 00/53211 and U.S. Pat. No. 5,981,568. The flow modulator (e.g., FGF2 or VEGFR3 agonist such as VEGF-c) and/or neurological therapeutic agent (e.g., amyloid beta antibody) can be administered in a pharmaceutical composition as described herein.

Targeted delivery of therapeutic compositions (comprising a flow modulator and/or neurological therapeutic agent as described herein) containing an antisense polynucleotide, expression vector (viral or non-viral), or subgenomic polynucleotides, or mRNA is also contemplated within the disclosure. Receptor-mediated DNA delivery techniques are described in, for example, Findeis et al., Trends Biotechnol. (1993) 11:202; Chiou et al., Gene Therapeutics: Methods and Applications of Direct Gene Transfer (J. A. Wolff, ed.) (1994); Wu et al., J. Biol. Chem. (1988) 263:621; Wu et al., J. Biol. Chem. (1994) 269:542; Zenke et al., Proc. Natl. Acad. Sci. USA (1990) 87:3655; Wu et al., J. Biol. Chem. (1991) 266:338. The nucleic acid can be delivered using any of the administration methods described herein. In one embodiment, the administration of a nucleic acid of the invention is parenteral, e.g., intravenous (e.g., as a bolus or as a diffusible infusion), intradermal, intraperitoneal, intramuscular, intrathecal, intraventricular, intracranial, intrathecal, subcutaneous, transmucosal, buccal, sublingual, endoscopic, rectal, oral, vaginal, topical, pulmonary, intranasal, urethral, or ocular. The route and site of administration may be chosen to enhance delivery or targeting of the nucleic acid of the invention to a particular location. For example, to target brain cells, intravenous, i.c.v, or ICM injection may be used.

The particular dosage regimen for any of the flow modulators and/or neurological therapeutic agents described herein, i.e., dose, timing and repetition, used in the methods of some embodiments herein depend on the particular subject and that subject's medical history.

In some embodiments, more than one flow modulator and/or neurological therapeutic agent, or a combination of a flow modulator and/or neurological therapeutic agent and another suitable therapeutic agent, may be administered to a subject in need of the treatment. The flow modulator (e.g., FGF2 and/or VEGFR3 agonist such as VEGF-c) and/or neurological therapeutic agent (e.g., amyloid beta antibody) can also be used in conjunction with other agents that serve to enhance and/or complement the effectiveness of the flow modulator and/or neurological therapeutic agent. In some embodiments, the flow modulator and/or neurological therapeutic agent is administered.

Treatment efficacy for a target neurological disease/disorder, for example a proteinopathy (e.g., a tauopathy and/or amyloidosis such as AD), for example in the head, skull, meninges, central nervous system, and/or brain as described herein can be assessed by methods well-known in the art. The target neurological disease/disorder can comprise amyloid beta plaques.

Pharmaceutical Compositions

A flow modulator (e.g., FGF2 or VEGFR3 agonist such as VEGF-c) and/or neurological therapeutic agent (e.g., amyloid beta antibody), as well as the encoding nucleic acids or nucleic acid sets, vectors comprising such, or host cells comprising the vectors, as described herein can be mixed with a pharmaceutically acceptable carrier (excipient) to form a pharmaceutical composition for use in treating a target disease, e.g., as described herein. “Acceptable” means that the carrier must be compatible with the active ingredient of the composition (and preferably, capable of stabilizing the active ingredient) and not deleterious to the subject to be treated. Pharmaceutically acceptable excipients (carriers) including buffers, which are well known in the art. See, e.g., Remington: The Science and Practice of Pharmacy 20th Ed. (2000) Lippincott Williams and Wilkins, Ed. K. E. Hoover.

“Therapeutically effective amount,” as used herein, is intended to include the amount of an agent or composition that is sufficient to effect treatment of the disease (e.g., by diminishing, ameliorating, or maintaining the existing disease or one or more symptoms of disease or its related comorbidities). The “therapeutically effective amount” may vary depending on the agent or composition, how it is administered, the disease and its severity and the history, age, weight, family history, genetic makeup, stage of pathological processes, the types of preceding or concomitant treatments, if any, and other individual characteristics of the patient to be treated.

“Prophylactically effective amount,” as used herein, is intended to include the amount of an agent or composition that, when administered to a subject who does not yet experience or display symptoms of a neurological disease, but who may be predisposed to a neurological disease, is sufficient to prevent or delay the development or progression of the disease or one or more symptoms of the disease for a clinically significant period of time. The “prophylactically effective amount” may vary depending on the agent or composition, how it is administered, the degree of risk of disease, and the history, age, weight, family history, genetic makeup, the types of preceding or concomitant treatments, if any, and other individual characteristics of the patient to be treated.

As used herein, “prevention” or “preventing,” when used in reference to a neurological disease, disorder or condition thereof, refers to a reduction in the likelihood that a subject will develop a symptom associated with such a neurological disease, disorder, or condition, e.g., a sign or symptom of a neurological disease.

An “effective amount” is intended to include a “therapeutically-effective amount” or “prophylactically effective amount.” An effective amount also includes an amount of an agent or composition that produces some desired local or systemic effect at a reasonable benefit/risk ratio applicable to any treatment. Agents and compositions employed in the methods of the present invention may be administered in a sufficient amount to produce a reasonable benefit/risk ratio applicable to such treatment. In some embodiments, a therapeutically effective amount or prophylactically effect amount tis administered in a single dose; in some embodiments, multiple unit doses are required to deliver a therapeutically or prophylactically effective amount.

As used herein “pharmaceutically acceptable carrier” has its ordinary meaning as understood in the art in view of the specification, and includes any and all solvents, dispersion media, coatings, antibacterial and antifungal agents, isotonic and absorption delaying agents, and the like. The use of such media and agents for pharmaceutically active substances is well known in the art. Except insofar as any conventional media or agent is incompatible with the active flow modulator and/or neurological therapeutic agent, use thereof in the therapeutic compositions is contemplated. Supplementary active compounds can also be incorporated into the compositions

The pharmaceutical compositions to be used in the present methods can comprise pharmaceutically acceptable carriers, excipients, or stabilizers in the form of lyophilized formulations or aqueous solutions. (Remington: The Science and Practice of Pharmacy 20th Ed. (2000) Lippincott Williams and Wilkins, Ed. K. E. Hoover). Acceptable carriers, excipients, or stabilizers are nontoxic to recipients at the dosages and concentrations used, and may comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG). Some additional examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations. The term “pharmaceutically-acceptable salts” refers to the relatively nontoxic, inorganic and organic acid addition salts of the agents encompassed by the disclosure. These salts can be prepared in situ during the final isolation and purification of the respiration uncoupling agents, or by separately reacting a purified respiration uncoupling agent in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like (See, for example, Berge et al. (1977) “Pharmaceutical Salts”, J. Pharm. Sci. 66: 1-19).

In other cases, the agents useful in the methods of the present disclosure may contain one or more acidic functional groups and, thus, are capable of forming pharmaceutically-acceptable salts with pharmaceutically-acceptable bases. The term “pharmaceutically-acceptable salts” in these instances refers to the relatively non-toxic, inorganic and organic base addition salts of such agents. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like (see, for example, Berge et al, supra).

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

The pharmaceutical compositions described herein can be in unit dosage forms such as tablets, pills, capsules, powders, granules, solutions or suspensions, or suppositories, for oral, parenteral or rectal administration, or administration by inhalation or insufflation.

Administration may be topical (including ophthalmic, vaginal, rectal, intranasal, transdermal), oral, or parenteral. Parenteral administration includes intravenous drip, subcutaneous, intraperitoneal or intramuscular injection, or intrathecal or intraventricular administration.

The route and site of administration may be chosen to enhance delivery or targeting of the flow modulator/and or neurological therapeutic agent of the invention to a particular location. For example, to target brain cells, intravenous, i.c.v, or ICM injection may be used.

Ophthalmic formulations, eye ointments, drops, powders, solutions and the like, are also contemplated as being within the scope of this disclosure.

Formulations for topical administration may include transdermal patches, ointments, lotions, creams, gels, drops, suppositories, sprays, liquids, and powders. Conventional pharmaceutical carriers, aqueous, powder or oily bases, thickeners and the like may be necessary or desirable. Coated condoms, gloves and the like may also be useful.

For topical applications, provided compositions or pharmaceutical compositions of some embodiments may be formulated in a suitable ointment containing the active component suspended or dissolved in one or more carriers. Carriers for topical administration of a therapeutic include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water. Compositions for oral administration include powders or granules, suspensions or solutions in water, syrups, elixirs or non-aqueous media, tablets, capsules, lozenges, or troches. In the case of tablets, carriers that can be used include lactose, sodium citrate and salts of phosphoric acid. Various disintegrants such as starch, and lubricating agents such as magnesium stearate, sodium lauryl sulfate and talc, are commonly used in tablets. For oral administration in capsule form, useful diluents are lactose and high molecular weight polyethylene glycols. When aqueous suspensions are required for oral use, the flow modulators and/or therapeutic agents described herein can be combined with emulsifying and suspending agents. If desired, certain sweetening or flavoring agents can be added.

When the flow modulator (e.g., FGF2 or VEGFR3 agonist such as VEGF-c) and/or neurological therapeutic agent (e.g., amyloid beta antibody) is suitably protected, as described herein, the flow modulator and/or neurological therapeutic agent can be orally administered, for example, with an inert diluent or an assimilable edible carrier.

In solid dosage forms for oral administration (capsules, tablets, pills, dragees, powders, granules and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof, 107 and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like. A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered peptide or peptidomimetic moistened with an inert liquid diluent.

Tablets, and other solid dosage forms, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and or microspheres. They may be sterilized by, for example, filtration through a bacteria-retaining filter, or by incorporating sterilizing agents in the form of sterile solid compositions, which can be dissolved in sterile water, or some other sterile injectable medium immediately before use. These compositions may also optionally contain opacifying agents and may be of a composition that they release the active ingredient(s) only, or in some embodiments, in a certain portion of the gastrointestinal tract, optionally, in a delayed manner. Examples of embedding compositions, which can be used include polymeric substances and waxes. The active ingredient can also be in micro-encapsulated form, if appropriate, with one or more of the above-described excipients.

For preparing solid compositions such as tablets, the principal active ingredient can be mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a compound of the present invention, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing from 0.1 to about 500 mg of the active ingredient of the present invention. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer that serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

Liquid dosage forms for oral administration include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof. Compositions for administration may include sterile aqueous solutions which may also contain buffers, diluents, and other suitable additives. Formulations for may include sterile aqueous solutions which may also contain buffers, diluents, and other suitable additives. For intravenous use, the total concentration of solutes may be controlled to render the preparation isotonic.

The flow modulator (e.g., FGF2 or VEGFR3 agonist such as VEGF-c) and/or neurological therapeutic agent (e.g., amyloid beta antibody) may also be administered parenterally or intraperitoneally. Dispersions can also be prepared in glycerol, liquid polyethylene glycols, and mixtures thereof, and in oils. Under ordinary conditions of storage and use, these preparations may contain a preservative to prevent the growth of microorganisms.

Pharmaceutical compositions of a flow modulator (e.g., FGF2 or VEGFR3 agonist such as VEGF-c) and/or neurological therapeutic agent (e.g., amyloid beta antibody) suitable for injectable use include sterile aqueous solutions (where water soluble) or dispersions and sterile powders for the extemporaneous preparation of sterile injectable solutions or dispersion. In all cases the composition will in some embodiments be sterile and must be fluid to the extent that the composition has easy syringeability (such as the composition easily passing from a container through an injection needle into a syringe prior to injection) and injectability (such as the composition easily passes from a syringe through an injection needle into an administration site of the subject)(See, e.g., Cilurzo et al. AAPS PharmSciTech. 12: 604-609 (2011) for a review of syringeability and injectability). It will in some embodiments be stable under the conditions of manufacture and storage and preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In some embodiments, include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Injectable compositions (such as pharmaceutical compositions comprising a flow modulator and/or neurological therapeutic agent) of some embodiments may contain various carriers such as vegetable oils, dimethylactamide, dimethyformamide, ethyl lactate, ethyl carbonate, isopropyl myristate, ethanol, and polyols (glycerol, propylene glycol, liquid polyethylene glycol, and the like). For intravenous injection, water soluble compositions comprising flow modulator (e.g., FGF2 or VEGFR3 agonist such as VEGF-c) and/or neurological therapeutic agent (e.g., amyloid beta antibody) can be administered by the drip method, whereby a pharmaceutical formulation containing the flow modulator and/or neurological therapeutic agent and a physiologically acceptable excipient is infused. Physiologically acceptable excipients may include, for example, 5% dextrose, 0.9% saline, Ringer's solution or other suitable excipients. Intramuscular preparations, e.g., a sterile formulation of a suitable soluble salt form of the flow modulator and/or neurological therapeutic agent, can be dissolved and administered in a pharmaceutical excipient such as Water-for-Injection, 0.9% saline, or 5% glucose solution. In some embodiments, the composition comprises VEGFR3 agonist and/or FGF2.

Sterile injectable solutions can be prepared by incorporating a flow modulator (e.g., FGF2 or VEGFR3 agonist such as VEGF-c) and/or neurological therapeutic agent (e.g., amyloid beta antibody) of the disclosure in the required amount in an appropriate solvent with one or a combination of ingredients enumerated above, as required, followed by filtered sterilization. Generally, dispersions are prepared by incorporating the active flow modulator and/or neurological therapeutic agent compound into a sterile vehicle which contains a basic dispersion medium and the required other ingredients from those enumerated above. In the case of sterile powders for the preparation of sterile injectable solutions, and, in some embodiments methods of preparation are vacuum drying and freeze-drying which yields a powder of the flow modulator and/or neurological therapeutic agent plus any additional desired ingredient from a previously sterile-filtered solution thereof

Suitable surface-active agents include, in particular, non-ionic agents, such as polyoxyethylenesorbitans (e.g., Tween™ 20, 40, 60, 80 or 85) and other sorbitans (e.g., Span™ 20, 40, 60, 80 or 85). Compositions with a surface-active agent will conveniently comprise between 0.05 and 5% surface-active agent, and can be between 0.1 and 2.5%. It will be appreciated that other ingredients may be added, for example mannitol or other pharmaceutically acceptable vehicles, if necessary.

Suitable emulsions may be prepared using commercially available fat emulsions, such as Intralipid™, Liposyn™, Infonutrol™, Lipofundin™ and Lipiphysan™. The active ingredient may be either dissolved in a pre-mixed emulsion composition or alternatively it may be dissolved in an oil (e.g., soybean oil, safflower oil, cottonseed oil, sesame oil, corn oil or almond oil) and an emulsion formed upon mixing with a phospholipid (e.g. egg phospholipids, soybean phospholipids or soybean lecithin) and water. It will be appreciated that other ingredients may be added, for example glycerol or glucose, to adjust the tonicity of the emulsion. Suitable emulsions will typically contain up to 20% oil, for example, between 5 and 20%.

The emulsion compositions can be those prepared by mixing an VEGFR3 agonist or VEGFR3 antagonist with Intralipid™ or the components thereof (soybean oil, egg phospholipids, glycerol and water).

Pharmaceutical compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as set out above. In some embodiments, the compositions are administered by the oral or nasal respiratory route for local or systemic effect.

Compositions in preferably sterile pharmaceutically acceptable solvents may be nebulized by use of gases. Nebulized solutions may be breathed directly from the nebulizing device or the nebulizing device may be attached to a face mask, tent or intermittent positive pressure breathing machine. Solution, suspension or powder compositions may be administered, preferably orally or nasally, from devices which deliver the formulation in an appropriate manner.

It is especially advantageous to formulate parenterally any of the flow modulators and/or neurological therapeutic agent compositions described herein in dosage unit form for ease of administration and uniformity of dosage. “Dosage unit form”, as used herein, has its ordinary meaning as understood in the art in view of the specification, and includes physically discrete units suited as unitary dosages for the mammalian subjects (such as humans) to be treated; each unit containing a predetermined quantity of active compound (e.g., flow modulator and/or neurological therapeutic agent) calculated to produce the desired therapeutic effect in association with the required pharmaceutical carrier. The specification for the dosage unit forms of the disclosure are dictated by, and directly dependent on, (a) the unique characteristics of the active flow modulator and/or neurological therapeutic agent and the particular therapeutic effect to be achieved, and (b) the limitations inherent in the art of compounding such an active flow modulator and/or neurological therapeutic agent for the treatment of sensitivity in individuals. In some embodiments, a flow modulator and/or neurological therapeutic agent of the disclosure is an antibody. As defined herein, a therapeutically effective amount of antibody (e.g., an effective dosage) ranges from about 0.001 to 30 mg kg body weight, in some embodiments about 0.01 to 25 mg kg body weight, in some embodiments about 0.1 to 20 mg kg body weight, and in some embodiments about 1 to 10 mg/kg, 2 to 9 mg/kg, 3 to 8 mg kg, 4 to 7 mg/kg, or 5 to 6 mg kg, or a range defined by any two of the preceding values, body weight. The skilled artisan will appreciate that certain factors may influence the dosage required to effectively treat a subject, including but not limited to the severity of the disease or disorder, previous treatments, the general health and/or age of the subject, and other diseases present.

Moreover, treatment of a subject with a therapeutically effective amount of a flow modulator (e.g., FGF2 or VEGFR3 agonist such as VEGF-c) and/or neurological therapeutic agent (such as an amyloid beta antibody) can include a single treatment or, in some embodiments, can include a series of treatments. In some embodiments, a subject is treated with antibody (such as a neurological therapeutic agent comprising, consisting essentially of, or consisting of an amyloid beta antibody) in the range of between about 0.1 to 20 mg kg body weight, one time per week for between about 1 to 10 weeks, in some embodiments between 2 to 8 weeks, in some embodiments between about 3 to 7 weeks, and in some embodiments for about 4, 5, or 6 weeks, or a range defined by any two of the preceding values. It will also be appreciated that the effective dosage of antibody (e.g., amyloid beta antibody) used for treatment may increase or decrease over the course of a particular treatment. Changes in dosage may result from the results of diagnostic assays. In addition, an antibody of the disclosure can also be administered in combination therapy with, e.g., chemotherapeutic agents, hormones, antiangiogens, radiolabeled, compounds, or with surgery, cryotherapy, and/or radiotherapy. An antibody of the disclosure (e.g., amyloid beta antibody) can also be administered in conjunction with additional forms of therapy (such as one or more conventional therapies, which may include, for example, an antibody, peptide, a fusion protein and/or small molecule), either consecutively with, pre- or post- the additional therapy. For example, the antibody can be administered with a therapeutically effective dose of chemotherapeutic agent. In some embodiment, the antibody can be administered in conjunction with chemotherapy to enhance the activity and efficacy of the chemotherapeutic agent. The Physicians' Desk Reference (PDR) discloses dosages of chemotherapeutic agents that have been used in the treatment of various neurological disease and disorders. The dosing regimen and dosages of these aforementioned chemotherapeutic drugs that are therapeutically effective will depend on the particular neurological disease or disorder, being treated, the extent of the disease and other factors familiar to the physician of skill in the art and can be determined by the physician.

In addition, the flow modulators (e.g., FGF2 or VEGFR3 agonists such as VEGF-c) and/or neurological therapeutic agents (e.g., amyloid beta antibodies) of the disclosure described herein can be administered using nanoparticle-based composition and delivery methods well known to the skilled artisan. For example, nanoparticle-based delivery for improved nucleic acid (e.g., small RNAs) therapeutics are well known in the art (Expert Opinion on Biological Therapy 7: 1811-1822).

A flow modulator (e.g., FGF2 or VEGFR3 agonist such as VEGF-c) and/or neurological therapeutic agent (e.g., amyloid beta antibody) can be administered to an individual in an appropriate carrier, diluent or adjuvant, co-administered with enzyme inhibitors or in an appropriate carrier such as liposomes. Pharmaceutically acceptable diluents include saline and aqueous buffer solutions. Adjuvant is used in its broadest sense and includes any immune stimulating compound such as interferon. Adjuvants contemplated herein include resorcinols, non-ionic surfactants such as polyoxyethylene oleyl ether and n-hexadecyl polyethylene ether. Enzyme inhibitors include pancreatic trypsin inhibitor, diisopropylfluorophosphate (DEEP) and trasylol. Liposomes include water-in-oil-in-water emulsions as well as conventional liposomes (Sterna et al. (1984) J. Neuroimmunol. 7:27).

In certain embodiments, the flow modulator and/or neurological therapeutic agent comprises a nucleic acid or polynucleotide. The nucleic acids or polynucleotides of the invention may include deoxynucleotides, ribonucleotides, modified deoxynucleotides, modified ribonucleotides (e.g., chemical modifications, such as modifications that alter the backbone linkages, sugar molecules, and/or nucleic acid bases), and artificial nucleic acids. In some embodiments, the polynucleotide includes, but is not limited to, genomic DNA, cDNA, peptide nucleic acids (PNA) or peptide oligonucleotide conjugates, locked nucleic acids (LNA), bridged nucleic acids (BNA), polyamides, triplex forming oligonucleotides, modified DNA, antisense DNA oligonucleotides, tRNA, mPvNA, rPvNA, modified RNA, miRNA, gRNA, and siRNA or other RNA or DNA molecules.

Therapeutic compositions containing a polynucleotide (e.g., a polynucleotide encoding a flow modulator and/or neurological therapeutic agent as described herein) may be administered in a range of about 100 ng to about 200 mg of DNA for local administration in a gene therapy protocol. In some embodiments, ranges of about 500 ng to about 50 mg, about 1 μg to about 2 mg, about 5 μg to about 500 μg, and about 20 μg to about 100 μg of DNA or more can also be used during a gene therapy protocol. In some embodiments, DNA is administered at a concentration of about 100 ng/ml to about 200 mg/ml.

The flow modulator (e.g., FGF2 and/or VEGFR3 agonists such as VEGF-c, described herein) and/or neurological therapeutic agent (e.g., amyloid beta antibody) can comprise, consist essentially of, or consist of one or more therapeutic polynucleotides and polypeptides, and can be delivered using gene delivery vehicles. The gene delivery vehicle can be of viral or non-viral origin (see generally, Jolly, Cancer Gene Therapy (1994) 1:51; Kimura, Human Gene Therapy (1994) 5:845; Connelly, Human Gene Therapy (1995) 1:185; and Kaplitt, Nature Genetics (1994) 6:148). Expression of such coding sequences can be induced using endogenous mammalian or heterologous promoters and/or enhancers. Expression of the coding sequence can be either constitutive or regulated.

In certain embodiments, a polynucleotide of the disclosure comprises a sequence that encodes a peptide or a protein, e.g., the lymphangiogenic growth factors and/or neurological therapeutic agent(s) disclosed herein, wherein the polynucleotide may be comprised in an expression vector. The polynucleotides of the invention, e.g., protein coding nucleic acid, may be expressed from transcription units inserted into DNA or RNA vectors (see, e.g., Couture, A, et al., TIG. (1996), 12:5-10; WO 00/22113, WO 00/22114, and U.S. Pat. No. 6,054,299). In some embodiments, expression is sustained (months or longer), depending upon the specific construct used and the target tissue or cell type. These transgenes can be introduced as a linear construct, a circular plasmid, or a viral vector, which can be an integrating or non-integrating vector. The transgene can also be constructed to permit it to be inherited as an extrachromosomal plasmid (Gassmann, et al., (1995) Proc. Natl. Acad. Sci. USA 92:1292).

The individual strand or strands of a polynucleotide encoding a flow modulator and/or a neurological therapeutic agent comprising a nucleic acid molecule can be transcribed from a promoter in an expression vector. Expression vectors are generally DNA plasmids or viral vectors. Expression vectors compatible with eukaryotic cells, preferably those compatible with vertebrate cells, can be used to produce recombinant constructs for the expression of a neurological therapeutic agent as described herein. Delivery of a flow modulater and/or a neurological therapeutic agent expressing vector can be systemic, such as by intravenous or intramuscular administration, by administration to target cells ex-planted from the patient followed by reintroduction into the patient, or by any other means that allows for introduction into a desired target cell, e.g., intracerebroventricularly (i.c.v.) or intra-cisterna magna (ICM), or any other method described herein.

Viral-based vectors for delivery of a desired polynucleotide (for example, encoding a flow modulator and/or neurological therapeutic agent as described herein) and expression in a desired cell are well known in the art. Exemplary viral-based vehicles include, but are not limited to, recombinant retroviruses (see, e.g., PCT Publication Nos. WO 90/07936; WO 94/03622; WO 93/25698; WO 93/25234; WO 93/11230; WO 93/10218; WO 91/02805; U.S. Pat. Nos. 5,219,740 and 4,777,127; GB Patent No. 2,200,651; and EP Patent No. 0 345 242), alphavirus-based vectors (e.g., Sindbis virus vectors, Semliki forest virus (ATCC VR-67; ATCC VR-1247), Ross River virus (ATCC VR-373; ATCC VR-1246) and Venezuelan equine encephalitis virus (ATCC VR-923; ATCC VR-1250; ATCC VR 1249; ATCC VR-532)), and adeno-associated virus (AAV) vectors (see, e.g., PCT Publication Nos. WO 94/12649, WO 93/03769; WO 93/19191; WO 94/28938; WO 95/11984 and WO 95/00655). AAV vectors are particularly suitable for delivery of a payload into the central nervous system. Over 100 AAV serotypes have been identified that differ in the binding capacity of capsid proteins to specific cell surface receptors that can transduce different cell types and brain regions in the CNS. Non-limiting examples of AAV serotypes include AAV1, AAV2/1, AAVDJ, AAV8, AAVDJ8, AAV9, and AAVDJ9.

Viral vector systems which can be utilized with the methods and compositions described herein include, but are not limited to, (a) adenovirus vectors; (b) retrovirus vectors, including but not limited to lentiviral vectors, moloney murine leukemia virus, etc.; (c) adeno-associated virus vectors; (d) herpes simplex virus vectors; (e) SV 40 vectors; (f) polyoma virus vectors; (g) papilloma virus vectors; (h) picornavirus vectors; (i) pox virus vectors such as an orthopox, e.g., vaccinia virus vectors or avipox, e.g. canary pox or fowl pox; and (j) a helper-dependent or gutless adenovirus. Replication-defective viruses can also be advantageous. Different vectors will or will not become incorporated into the cells' genome. The constructs can include viral sequences for transfection, if desired. Alternatively, the construct can be incorporated into vectors capable of episomal replication, e.g. EPV and EBV vectors. Constructs for the recombinant expression of a protein of the invention will generally require regulatory elements, e.g., promoters, enhancers, etc., to ensure the expression of the protein in target cells. Other aspects to consider for vectors and constructs are known in the art.

Vectors, including those derived from retroviruses such as lentivirus, are suitable tools to achieve long-term gene transfer since they allow long-term, stable integration of a transgene and its propagation in daughter cells. Examples of vectors include expression vectors, replication vectors, probe generation vectors, and sequencing vectors. The expression vector may be provided to a cell in the form of a viral vector. Viral vector technology is well known in the art, and described in a variety of virology and molecular biology manuals. Viruses, which are useful as vectors include, but are not limited to, retroviruses, adenoviruses, adeno-associated viruses, herpes viruses, and lenti viruses. In general, a suitable vector contains an origin of replication functional in at least one organism, a promoter sequence, convenient restriction endonuclease sites, and one or more selectable markers.

Administration of DNA linked to killed adenovirus as described in Curiel, Hum. Gene Ther. (1992) 3:147 can also be employed.

In some embodiments, non-viral delivery vehicles and methods are employed, including, but not limited to, polycationic condensed DNA linked or unlinked to killed adenovirus alone (see, e.g., Curiel, Hum. Gene Ther. (1992) 3:147); ligand-linked DNA (see, e.g., Wu, J. Biol. Chem. (1989) 264:16985); eukaryotic cell delivery vehicles cells (see, e.g., U.S. Pat. No. 5,814,482; PCT Publication Nos. WO 95/07994; WO 96/17072; WO 95/30763; and WO 97/42338) and nucleic charge neutralization or fusion with cell membranes. Naked DNA can also be employed. Exemplary naked DNA introduction methods are described in PCT Publication No. WO 90/11092 and U.S. Pat. No. 5,580,859. Liposomes that can act as gene delivery vehicles are described in U.S. Pat. No. 5,422,120; PCT Publication Nos. WO 95/13796; WO 94/23697; WO 91/14445; and EP Patent No. 0524968. Additional approaches are described in Philip, Mol. Cell. Biol. (1994) 14:2411, and in Woffendin, Proc. Natl. Acad. Sci. (1994) 91:1581.

The expression vector to be introduced can also contain either a selectable marker gene or a reporter gene or both to facilitate identification and selection of expressing cells from the population of cells sought to be infected or transfected through viral or non-viral vectors. In other aspects, the selectable marker may be carried on a separate piece of DNA and used in a co-transfection procedure. Both selectable markers and reporter genes may be flanked with appropriate transcriptional control sequences to enable expression in the host cells. Useful selectable markers include, for example, antibiotic-resistance genes, such as neo and the like.

Reporter genes may be used for identifying potentially transfected cells and for evaluating the functionality of transcriptional control sequences. In general, a reporter gene is a gene that is not present in or expressed by the recipient source and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription

Expression of natural or synthetic nucleic acids is typically achieved by operably linking a nucleic acid encoding the gene of interest to a promoter, and incorporating the construct into an expression vector. The vectors can be suitable for replication and integration in eukaryotes. Typical cloning vectors contain transcription and translation terminators, initiation sequences, and promoters useful for expression of the desired nucleic acid sequence.

Additional promoter elements, e.g., enhancing sequences, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

Promoters to drive expression in the brain can be constitutive, such as beta-actin, phosphoglycerate kinase 1 or CMV promoters, or tissue specific. Examples of tissue specific promoters which can be used to drive expression in brain tissues include the synapsin, Glial fibrillary acidic protein (GFAP), glutamic acid decarboxylase (GAD67), homeobox Dlx5/6, glutamate receptor 1 (GluR1), and preprotachykinin 1 (Tac1), and Musashi1 promoters. These promoters show diversity of transcriptional activity and cell-type specificity of expression. Accordingly, in some embodiments, a promoter is selected based on the desired expression in a cell type, tissue or brain region. Accordingly, in some embodiments, the expression of the nucleic acid of the interest, e.g., encoding a flow modulator and/or neurological therapeutic agent as described herein, is under control of a brain tissue specific promoter, including but not limited to, synapsin, GFAP, GAD67, homeobox Dlx5/6, GluR1, and Tac1, and Musashi1 promoters or others known in the art.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-la (EF-la). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter.

Further, the present invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

In some embodiments, the flow modulator (e.g., FGF2 or VEGFR3 agonist such as VEGF-c) and/or neurological therapeutic agent (e.g., amyloid beta antibody) is administered as an mRNA (e.g., a mRNA encoding the VEGFR3 agonist). In some embodiments, chemically modified messenger RNA (mRNA) is employed. Modified mRNA evades recognition by the innate immune system and is less immunostimulating than dsDNA or regular mRNA. Additionally, cytoplasmic delivery of mRNA circumvents the nuclear envelope, which can result in a higher expression level. Exemplary mRNA introduction methods are described in Rhoads et al. (Methods in Molecular Biology, vol. 1428, DOI 10.1007/978-1-4939-3625-01), and references therein.

In some embodiments, nucleic acids (e.g., comprising or encoding a flow modulator and/or neurological therapeutic agent) are delivered to a cell naked, i.e., free from complexing agents, for example, lipid agents and polymer agents, etc. In some embodiments, naked mRNA is delivered by injection (intradermal, intrathecal, intraventricular, intraparenchymal, etc).

The flow modulator and/or neurological therapeutic agent nucleic acids or polypeptides of compositions and methods of some embodiments may be formulated according to methods known in the art, and the formulations may further include, but are not limited to, cell penetration agents, a pharmaceutically acceptable carrier, delivery agents, a bioerodible or biocompatible polymers, solvents, and sustained-release delivery depots.

In some embodiments, the nucleic acid comprising or encoding the flow modulator and/or neurological therapeutic agent (e.g., RNAi agent, siRNA, ASO, LNA, or mRNA, or DNA) comprises, consists essentially of, or consists of modified nucleic acids or nucleobases. Example flow modulators that can be encoded include VEGFR3 agonist or antagonist, or FGF2. Example neurological therapeutic agents include amyloid beta antibodies. In some embodiments, the nucleic acid encoding the flow modulator and/or neurological therapeutic agent comprises, consists essentially of, or consists of an antisense oligonucleotide (ASO). By way of example, the ASO can hybridize to a complementary mRNA and mediate silencing of expression from the mRNA, such as by blocking ribosome binding to the mRNA and/or recruiting RNase H to mediate degradation of the mRNA. For example, the nucleic acid molecule can be a mimetic, can include a modified sugar backbone, one or more modified internucleoside linkages (e.g., one or more phosphorothioate and/or heteroatom internucleoside linkages), one or more modified bases, and the like. In some embodiments, the nucleic acid has a morpholino backbone structure. In some embodiments, the nucleic acid has one or more locked nucleic acids (LNAs). Suitable sugar substituent groups include methoxy (—O—CH3), aminopropoxy (—O CH2 CH2 CH2NH2), allyl (—CH2-CH═CH2), —O-allyl (—O—CH2-CH═CH2) and fluoro (F). 2′-sugar substituent groups may be in the arabino (up) position or ribo (down) position. In some embodiments, the nucleic acid has base modifications. Base modifications include synthetic and natural nucleobases. Suitable base modifications include pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methylpseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, 4-methoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, 2-aminopurine, 2,6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, 2-methoxy-adenine, inosine, 1-methylinosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine. In some embodiments, the flow modulator and/or neurological therapeutic agent can comprise naturally occurring and/or artificial nucleic acid, for example a mimetic, one or more modified internucleoside linkages, and/or one or more modified bases, such as base modifications as described herein.

In some embodiments, the nucleic acid (e.g., of the flow modulator and/or neurological therapeutic agent) is an mRNA which has at least one modification in one of the bases A, G, U and/or C. In some embodiments, the mRNA has at least one 5′ terminal cap is selected from the group consisting of Cap0, Cap 1, ARCA, inosine, N1-methyl-guanosine, 2′fluoro-guanosine, 7-deaza-guanosine, 8-oxo-guanosine, 2-amino-guanosine, LNA-guanosine and 2-azido-guanosine. In some embodiments the mRNA has a polyA or a polyT tail, for example, of 100-200 nucleotides. In some embodiments, the comprises a 5′ untranslated region and/or a 3′ untranslated region. In some examples, the UTR(s) are not derived from the native untranslated region corresponding to the polypeptide of interest, e.g., are beta-globin UTRs. In some embodiments, the sequence composition of the mRNA is altered by incorporating the most GC-rich codon for each amino acid. For example, expression of proteins from synthetic mRNA can be diminished if the mRNA contains rare codons or rate-limiting regulatory sequences. Redesign of the mRNA by using synonymous but more frequently used codons can increase the rate of translation and hence, translational yield (Gustafsson et al., Trends Biotechnol 22:346-353). In some embodiments, the nucleic acids of the disclosure are codon optimized, e.g., to optimize translation and reduce immunogenicity. Codon optimization tools known in the art employ algorithms for codon optimization, many of which take codon usage tables, codon adaptability, mRNA structure, and various cis-elements in transcription and translation into consideration. A non-limiting example of a useful platform is ptimumGene™ algorithm from GenScript.

Structural modifications to mRNA (such as flow modulator and/or neurological therapeutic agent mRNA) increase stability and translational efficiency can be divided into the various domains of mRNA: cap, UTRs, coding region, poly(A) tract, and 3′-end. mRNA is produced according to methods known in the art (see e.g., Rhoads (ed.), Synthetic mRNA: Production, Introduction Into Cells, and Physiological Consequences, Methods in Molecular Biology, vol. 1428, DOI 10.1007/978-1-4939-3625-0_1). mRNA is synthesized in vitro, e.g., using T7 polymerase-mediated transcription from a linearized DNA template containing an open reading frame, flanking 5′ and 3′ untranslated regions and a poly-A tail. A Cap structure, such as a Cap1 structure, can be enzymatically added to the 5′ end to produce the final mRNA. In some embodiments, the nucleobases bases are modified. For example, in some embodiments, uridine is completely substituted with N1-methylpseudouridine to reduce potential immunostimulatory activity and to improve protein expression relative to unmodified mRNA. Alternatively, one or more of the modifications described supra can be used. After the mRNA is purified, the mRNA is diluted, frozen or prepared for administration. Suitable buffer solutions are known in the art. A non-limiting example of a suitable buffer is a solution containing 2.94 mg/mL sodium citrate dihydrate at pH 6.5 and 7.6 mg/mL sodium chloride (Gan et al., Nature Communications, volume 10, Article number: 871 (2019)).

In some embodiments, the nucleic acid (e.g., comprising or encoding a flow modulator and/or neurological therapeutic agent) includes a conjugate moiety (e.g., one that enhances the activity, cellular distribution or cellular uptake of the oligonucleotide). These moieties or conjugates can include conjugate groups covalently bound to functional groups such as primary or secondary hydroxyl groups. Conjugate groups include, but are not limited to, intercalators, reporter molecules, polyamines, polyamides, polyethylene glycols, polyethers, groups that enhance the pharmacodynamic properties of oligomers, and groups that enhance the pharmacokinetic properties of oligomers. Suitable conjugate groups include, but are not limited to, cholesterols, lipids, phospholipids, biotin, phenazine, folate, phenanthridine, anthraquinone, acridine, fluoresceins, rhodamines, coumarins, and dyes. Groups that enhance the pharmacodynamic properties include groups that improve uptake, enhance resistance to degradation, and/or strengthen sequence-specific hybridization with the target nucleic acid. Groups that enhance the pharmacokinetic properties include groups that improve uptake, distribution, metabolism or excretion of a nucleic acid

The methods to deliver a polynucleotide or a nucleic acid to a cell are known in that art. The delivery of the nucleic acid neurological therapeutic agent of the disclosure to a cell e.g., a cell within a subject, such as a human subject (e.g., a subject in need thereof, such as a subject having a neurodegenerative disease, e.g., Alzheimer's disease) may be achieved in a number of different ways. For example, delivery may be performed by contacting a cell with a nucleic acid of the disclosure either in vitro, ex vivo, or in vivo. In vivo delivery may be performed directly by administering a composition comprising a peptide or protein neurological therapeutic agent to a subject. Alternatively, in vivo delivery may be performed indirectly by administering one or more vectors that encode and direct the expression of the neurological therapeutic agent. These alternatives are discussed further below.

In general, any method of delivery of a nucleic acid of the disclosure (in vitro, ex vivo, or in vivo) may be adapted for use with the nucleic acid of the disclosure (see e.g., Akhtar S. and Julian R L., (1992) Trends Cell. Biol. 2(5):139-144 and WO94/02595, which are incorporated herein by reference in their entireties). For in vivo delivery, factors to be considered for delivering a nucleic acid of the disclosure include, for example, biological stability of the neurological therapeutic agent, prevention of non-specific effects, and accumulation of the neurological therapeutic agent in the target tissue. The non-specific effects of a neurological therapeutic agent can be minimized by local administration, for example, by direct injection or implantation into a tissue or topically administering a composition comprising the neurological therapeutic agent. Local administration to a treatment site maximizes local concentration of the neurological therapeutic agent, limits the exposure of the neurological therapeutic agent to systemic tissues that can otherwise be harmed by the neurological therapeutic agent or that can degrade the neurological therapeutic agent, and permits a lower total dose of the neurological therapeutic agent to be administered.

For administering a nucleic acid of the disclosure systemically for the treatment of a disease, such as a neurodegenerative disease, the nucleic acid, e.g., a nucleic acid encoding a flow modulator, such as VEGFC, or a nucleic acid encoding an antibody specifically binding Tau protein, can be modified or alternatively delivered using a drug delivery system; both methods act to prevent the rapid degradation of the nucleic acid molecule by endo- and exo-nucleases in vivo. Modification of a nucleic acid molecule also permits targeting of the nucleic acid to a target tissue and avoidance of undesirable off-target effects. For example, a nucleic acid molecule of the disclosure may be modified by chemical conjugation to lipophilic groups such as cholesterol to enhance cellular uptake and prevent degradation.

Alternatively, a nucleic acid of the disclosure may be delivered using a drug delivery system such as a nanoparticle, a dendrimer, a polymer, a liposome, or a cationic delivery system. Positively charged cationic delivery systems facilitate binding of nucleic acid molecule (e.g., negatively charged molecule) and also enhance interactions at the negatively charged cell membrane to permit efficient uptake of a nucleic acid molecule by the cell. Cationic lipids, dendrimers, or polymers can either be bound to a nucleic acid, or induced to form a vesicle or micelle (see e.g., Kim S H. et al., (2008) Journal of Controlled Release 129(2):107-116) that encases the neurologic therapeutic agent. The formation of vesicles or micelles further prevents degradation of the neurological therapeutic agent when administered systemically. Methods for making and administering cationic complexes are well within the abilities of one skilled in the art (see e.g., Sorensen, D R., et al. (2003) J. Mol. Biol 327:761-766; Verma, U N. et al., (2003) Clin. Cancer Res. 9:1291-1300; Arnold, A S et al. (2007) J. Hypertens. 25:197-205, which are incorporated herein by reference in their entirety). Some non-limiting examples of drug delivery systems useful for systemic delivery of a nucleic acid of the disclosure include DOTAP (Sorensen, D R., et al (2003), supra; Verma, U N. et al., (2003), supra), Oligofectamine, “solid nucleic acid lipid particles” (Zimmermann, T S. et al., (2006) Nature 441:111-114), cardiolipin (Chien, P Y. et al., (2005) Cancer Gene Ther. 12:321-328; Pal, A. et al., (2005) Int J Oncol. 26:1087-1091), polyethyleneimine (Bonnet M E. et al., (2008) Pharm. Res. August 16 Epub ahead of print; Aigner, A. (2006) J Biomed. Biotechnol. 71659), Arg-Gly-Asp (RGD) peptides (Liu, S. (2006) Mol. Pharm. 3:472-487), and polyamidoamines (Tomalia, D A. et al., (2007) Biochem. Soc. Trans. 35:61-67; Yoo, H. et al., (1999) Pharm. Res. 16:1799-1804). In some embodiments, a nucleic acid (e.g., DNA, or mRNA) forms a complex with cyclodextrin for systemic administration. Methods for administration and pharmaceutical compositions comprising cyclodextrins may be found in U.S. Pat. No. 7,427,605, the entire contents of which are incorporated herein by reference.

In some examples, the pharmaceutical composition described herein comprises liposomes containing the flow modulator (e.g., FGF2 or VEGFR3 agonist such as VEGF-c) and/or neurological therapeutic agent (e.g., amyloid beta antibody) (or the encoding nucleic acids) which can be prepared by methods known in the art, such as described in Epstein, et al., Proc. Natl. Acad. Sci. USA 82:3688 (1985); Hwang, et al., Proc. Natl. Acad. Sci. USA 77:4030 (1980); and U.S. Pat. Nos. 4,485,045 and 4,544,545. Liposomes with enhanced circulation time are disclosed in U.S. Pat. No. 5,013,556. Particularly useful liposomes can be generated by the reverse phase evaporation method with a lipid composition comprising phosphatidylcholine, cholesterol and PEG-derivatized phosphatidylethanolamine (PEG-PE). The liposomes can be extruded through filters of defined pore size to yield liposomes with the desired diameter. Without being limited by theory, it is contemplated that minimizing lipid diameter can inhibit or avoid interaction between the liposome and circulating proteins, thus prolonging the circulation time of the liposome. Lipsomes for mRNA delivery reviewed, for example, in Reichmuth et al., Ther. Deliv. 7: 319-334 (2016), which is incorporated by reference in its entirety herein. In some embodiments, the liposome has a diameter smaller than the interior diameter of a meningeal lymphatic vessel, so that the liposome may travel through the meningeal lymphatics. In some embodiments, the liposome has a dimeter of less than 150 nm, for example, less than 140 nm, 130 nm, 120 nm, 110 nm, 100 nm, 90 nm, 80 nm, 70 nm, 60 nm, 50 nm, 40 nm, 30 nm, 20 nm, or 10 nm, including ranges between any two of the listed values. Without being limited by theory, it is contemplated that a target cell can endocytose of a liposome comprising mRNA, and following release of the mRNA from the lipsome, the mRNA can be available in the cytosol of the target cell. Without being limited by theory, it is contemplated that the inclusion of an amine group at or near the surface of the liposome can maintain a neutral or mildly cationic surface charge at physiological pH, so as to minimize non-specific protein interactions can facilitate release of the mRNA in the cytosol. In some embodiments, the liposomes are administered to a subject in vivo according to a route of administration as described herein, for example parenteral or intranasal.

In some embodiments, the flow modulator (e.g., FGF2 or VEGFR3 agonist such as VEGF-c) and/or neurological therapeutic agent (e.g., amyloid beta antibody) mRNA is contained in 3β-[N—(N′,N′-dimethylaminoethane) carbamoyl](DC-Cholesterol)/dioleoylphosphatidylethanolamine (DOPE) liposomes. In some embodiments, the VEGFR3 agonist is encapsulated with DOTAP. In some embodiments, the VEGFR3 agonist is encapsulated in a cationic lipid preparation. RNA can also be protected against degradation by complexing with the polycationic protein protamine. Accordingly, in some embodiments, VEGFR3 agonist mRNA is complexed with protamine, for example, according to the curevac RNActive® platform. Without being limited by theory, complexing with protamine is contemplated to inhibit or limit immunogenicity of the composition comprising the mRNA.

Excipients suitable for flow modulators (e.g., FGF2 or VEGFR3 agonist such as VEGF-c) and/or neurological therapeutic agents (e.g., amyloid beta antibody) as described herein can include, without limitation, lipidoids, liposomes, lipid nanoparticles, polymers, lipoplexes, core-shell nanoparticles, peptides, proteins, carbohydrates, cells loaded with nucleic acids or polypeptides of the disclosure, hyaluronidase, nanoparticle mimics and combinations thereof. In some embodiments, nucleic acids can be delivered using a Gene Gun.

In some embodiments, nucleic acids, e.g., DNA, mRNA, siRNA, etc., may be formulated in lipidoids. Non-limiting examples of such lipidoids contain amino-alkyl-acrylate and -acrylamide materials and are known in the art (see e.g., Love et al, PNAS May 25, 2010 107 (21) 9915). C16-96, C14-110, and C12-200 are other examples of lipidoids, which can be prepared, complexed with nucleic acid, e.g., siRNA or mRNA, and delivered according to Love et al, PNAS May 25, 2010 107 (21) 9915.

In some embodiments, the nucleic acid of the disclosure is administered in stable nucleic acid lipid particle (SNALP) formulations.

In one embodiment, nucleic acids or polypeptides described herein may be formulated in lipid nanoparticles. The formulation may be influenced by parameters including, but not limited to, the selection of the cationic lipid component, the degree of cationic lipid saturation, the nature of the PEGylation, ratio of all components and biophysical parameters such as size (Semple et al. Nature Biotech. 2010 28 172-176). A non-limiting example of a cationic lipid that is suitable for formulation of nucleic acids, e.g., mRNA, 1,2-dilinoleyloxy-3-dimethylaminopropane (DLinDMA). This cationic lipid can be synthesized and used as a main component of lipid nanoparticles. An ethanol dilution process is used to produce small uniform lipid particles with a high RNA encapsulation efficiency (Geall et al., PNAS Sep. 4, 2012 109 (36) 14604-14609).

In one embodiment, nucleic acids or polypeptides described herein may be formulated in exosomes, microvesicles, and/or extracellular vesicles. The exosomes, microvesicles, and/or extracellular vesicles may be loaded with at least one VEGFR3 agonist or VEGFR3 antagonist and delivered to cells or tissues. Exosomes which can function as nucleic acid delivery vehicles are known in the art and are for example described in U.S. Pat. No. 9,629,929.

The flow modulator (e.g., FGF2 or VEGFR3 agonist such as VEGF-c) and/or neurological therapeutic agent (e.g., amyloid beta antibody), or the encoding nucleic acid(s), may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are known in the art, see, e.g., Remington, The Science and Practice of Pharmacy 20th Ed. Mack Publishing (2000).

In other examples, the pharmaceutical composition described herein can be formulated in sustained-release format. Suitable examples of sustained-release preparations include semipermeable matrices of solid hydrophobic polymers containing the polypeptide or nucleic acid of the disclosure, which matrices are in the form of shaped articles, e.g. films, or microcapsules. Examples of sustained-release matrices include polyesters, hydrogels (for example, poly(2-hydroxyethyl-methacrylate), or poly(vinyl alcohol)), polylactides (U.S. Pat. No. 3,773,919), copolymers of L-glutamic acid and 7 ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), sucrose acetate isobutyrate, and poly-D-(−)-3-hydroxybutyric acid).

In some embodiments, the compositions described herein may include at least one polymer such as, but not limited to, polyethenes, polyethylene glycol (PEG), poly(l-lysine)(PLL), PEG grafted to PLL, cationic lipopolymer, biodegradable cationic lipopolymer, polyethylenimine (PEI), cross-linked branched poly(alkylene imines), a polyamine derivative, a modified poloxamer, a biodegradable polymer, elastic biodegradable polymer, biodegradable block copolymer, biodegradable random copolymer, biodegradable polyester copolymer, biodegradable polyester block copolymer, biodegradable polyester block random copolymer, multiblock copolymers, linear biodegradable copolymer, poly[a-(4-aminobutyl)-L-glycolic acid) (PAGA), biodegradable cross-linked cationic multi-block copolymers, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, polyamines, polylysine, poly(ethylene imine), poly(serine ester), poly(L-lactide-co-L-lysine), poly(4-hydroxy-L-proline ester), acrylic polymers, amine-containing polymers, dextran polymers, dextran polymer derivatives or combinations thereof.

A self-assembling polyplex nanomicelle composed of a polyethylene glycol-polyamino acid block copolymer was used to administer luciferase-expressing mRNA with nucleoside modification into the CNS by intrathecal injection into the cisterna magna of mice (Ushida et al., PLoS One 8, e56220(2013). Accordingly, in some embodiments, the nucleic acid, e.g., mRNA, of the disclosure is delivered intrathecally in a polyplex nanomicelle composed of a polyethylene glycol-polyamino acid block copolymer.

The pharmaceutical compositions of some embodiments herein are to be used for in vivo administration, and are sterile. Sterilization can be readily accomplished by, for example, filtration through sterile filtration membranes. Therapeutic flow modulator and/or neurological therapeutic agent compositions are generally placed into a container having a sterile access port, for example, an intravenous solution bag or vial having a stopper pierceable by a hypodermic injection needle.

The nucleic acid of the disclosure may be incorporated into pharmaceutical compositions suitable for administration. Such compositions typically include one or more species of nucleic acid and a pharmaceutically acceptable carrier including solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions. The pharmaceutical compositions comprising polynucleotides may be administered by any of the administration methods described herein.

Neurological Therapeutic Agents

The present disclosure provides therapy or treatment of neurodegenerative diseases using neurological therapeutic agents in combination with flow modulators. The neurological therapeutic agent (e.g., an amyloid beta antibody) surprisingly synergizes with the flow modulator (e.g., VEGFR3 agonist and/or FGF2) to improve the treatment of a subject. Without wishing to be bound by theory, flow modulators increase fluid flow in the central nervous system (e.g., brain) and as a result improve the delivery of the neurological therapeutic agents and/or drainage of the lymphatic vessel within the central nervous system (e.g., brain). In one non-limiting example, improved drainage allows the removal of pathological aggregates targeted by the therapeutic agent. Accordingly, without wishing to be bound by theory, a treatment combining a flow modulator described herein with a therapeutic agent described herein may improve the extent of the desired effect on the pathology of the neurological disease, e.g., reduction of pathological protein aggregates, reduction of inflammation at the site of aggregation, etc., as compared to a treatment with the therapeutic agent alone or as compared to a treatment with the flow modulator alone.

As used herein, a “neurological therapeutic agent” refers to an agent that treats, prevents, inhibits, ameliorates, or reduces the symptoms of one or more neurological diseases, for example proteinopathies as described herein (e.g., tauopathies and/or amyloidoses such as AD). In certain embodiments, the neurological therapeutic agent is selected from a group consisting of a small molecule, an oligopeptide, a polypeptide, an antibody, a nucleic acid, a recombinant virus, a vaccine, and a cell.

The neurological therapeutic agent may target a peptide or a protein that is involved in the pathogenesis of a neurologic disease. The exemplary target peptides or proteins include, but are not limited to, Aβ (Amyloid-β peptide), synuclein, fibrin, tau, apolipoprotein E (ApoE), TDP43, prion protein, Huntingtin exon 1, ABri peptide, ADan peptide, fragments of immunoglobulin light chains, fragments of immunoglobulin heavy chains, full or N-terminal fragments of serum amyloid A protein (SAA), transthyretin (TTR), β₂-microglobulin, N-terminal fragments of apolipoproteinA-I (ApoAI), C-terminal extended apolipoprotein A-II (ApoAII), N-terminal fragments of apolipoprotein A-IV (ApoAIV), apolipoprotein C-II (ApoCII), apolipoprotein C-III (ApoAIII), fragments of gelsolin, lysozyme, fragments of fibrinogen α-chain, N-terminal truncated cystatin C, islet amyloid polypeptide (IAPP), calcitonin, atrial natriuretic factor (ANF), N-terminal fragments of prolactin (PRL), insulin, medin, lactotransferrin, odontogenic ameloblast-associated protein (ODAM), pulmonary surfactant-associated protein C (SP-C), leukocyte cell-derived chemotaxin-2 (LECT-2), galectin 7 (Gal-7), Corneodesmosin (CDSN), C-terminal fragments of kerato-epthelin (βih-h3), semenogelin-1 (SGI), proteins S100A8/A9, Enfuvirtide, GSK-3β, MARK, CDK5, tyrosine kinase Fyn, protein phosphatase 2A (PP2A), LRRK2, GBA, NF-κB p65 (see, Chiti et al., Protein Misfolding, Amyloid Formation, and Human Disease: A Summary of Progress Over the Last Decade, Annu. Rev. Biochem., 86:35.1-35.42 (2017)).

The neurological therapeutic agent may target a peptide or a protein specifically or non-specifically. As used herein, the terms “specific targeting” or “specifically targets” refer to an ability to discriminate between possible peptides or proteins in the environment in which the interaction between the neurological therapeutic agent and the target is to occur. In some embodiments, a neurological therapeutic agent that interacts, e.g., preferentially interacts, with one particular peptide or protein when other potential neurological therapeutic agents are present is said to “specifically target” the peptide or protein with which it interacts. In some embodiments, specific targeting is assessed by detecting or determining the degree of association between the neurological therapeutic agent and its target. In some embodiments, specific targeting is assessed by detecting or determining ability of the neurological therapeutic agent to compete with an alternative interaction between its target and another entity. In some embodiments, specific targeting is assessed by performing such detections or determinations across a range of concentrations. Exemplary specific targeting includes, but is not limited to, specific binding of an antibody to its target protein. Exemplary non-specific targeting includes, but is not limited to, the interaction between a small molecule and a class of proteins or peptides. For example, a tyrosine kinase inhibitor may non-specifically inhibit several protein tyrosine kinases. In some embodiments, a neurological therapeutic agent of the invention may modulate, e.g., increase or decrease, the activity (e.g., enzyme activity) of its target protein or proteins. In some embodiments, a neurological therapeutic agent may modulate, e.g., increase or decrease, the folding and/or aggregation a peptide or protein. For example, a neurological therapeutic agent may binds to a protein, e.g., amyloid-3 or tau protein, to reduce the misfolding and aggregation thereof.

A neurological therapeutic agent may modulate a target peptide or protein directly or indirectly. For example, by direct modulation, the neurological therapeutic agent may interact with the target peptide or protein directly, e.g., an antibody binds to the target peptide or protein. In some other examples, the neurological therapeutic agent may indirectly modulate the target peptide or protein by interacting with another peptide or protein, and modulate the target by interacting with the another peptide or protein. For example, a protein kinase inhibitor may interact with a protein kinase to indirectly modulate the phosphorylation status of the target peptide or protein.

A neurological therapeutic agent may also not target a peptide or protein that is involved in the genesis of a proteinopathy. For example, a neurological therapeutic agent of the invention may prevent or reduce the downstream event of the proteinopathy, such as neuroinflammation that is associated with a neurodegenerative disease.

In certain embodiments, the neurological therapeutic agent comprises a small molecule. The term “small molecule,” as used herein, refers to a low molecular weight (e.g., <900 daltons) organic compound. Exemplary small molecules that can be used as neurological therapeutic agents include, but are not limited to, the small molecular agent listed in Table 2.

TABLE 2

| Donepezil | Galantamine | Rivastigmine | Memantine | Lanabecestat | Atabecestat |
|---|---|---|---|---|---|
| Verubecestat | Elenbecestat | Semagacestat | Tarenflurbil | Brexipiprazole | AXS-05 |
| | | | | | (Axsome |
| | | | | | Therapeutics) |
| AC-1204 | masitinib | amilomotide | guanfacine | octohydroaminoacridine | lumateperone |
| (Accera) | | | hydrochloride | succinate | tosylate |
| AVP-786 | ALZT-OP1 | AZD-1080 | ASN 120290 | GV-971 | CNP520 |
| (Avanir | | (AstraZeneca) | (Asceneuron | | (Novartis) |
| Pharma- | | | SA) | | |
| ceuticals) | | | | | |
| DNL104(RIP | DNL747 (RIPK1 | Namzaric | Namenda XR | Reminyl | tideglusib |
| K1 inhibitor) | inhibitor) | | | | (NP031112 |
| NP-12) | saracatinib | NPT200-11 | NPT088 | NPT100-18A | methylene |
| | (AZD0530) | | | thiamet-G | blue |
| LMTX | SEL-141 (Selvita | TauRx | nilvadipine | AZPZs | AZP2006 |
| (leuco- | SA) | Therapeutics) | | | |
| methylthionium | | | | | |
| bis (hydro- | | | | | |
| methanesulfonate | | | | | |
| (LMTM) | | | | | |
| and leuco- | | | | | |
| methylthionium | | | | | |
| dihydrobromide | | | | | |
| (LMTB) | | | | | |
| RPEL | PE859 | SLM | SLOH | IMS-088 (ImStar) | derivatives of |
| | | | | | 2 |
| 4- | rhein-huprine | 1- | apomorphine | carbenoxolone | trazodone |
| thiazolidinedione | hydrid | benzylamino- | | | |
| | | 2- | | | |
| | | hydroxyalkyl | | | |
| | | derivatives | | | |
| hexachlorophene | rifaximin | memantine | rotigotine ER | Duopa (AbbVie) | donepezil |
| | | hydrochloride | | | hydrochloride |
| Madopar | droxidopa | pimavanserin | deutetrabenazine | zonisamide | cysteamine |
| (Taiyo | | tartrate | | | |
| Pharma) | | | | | |
| galantamine | tetrabenazine | Stalevo | ropinirole | Rytary (Amneal | istradefylline |
| hydrobromide | | (Novartis) | hydrochloride | Pharmaceuticals) | |
| apomorphine | cerliponase alfa | amantadine | idebenone | bromocriptine | Neodopasol |
| hydrochloride | | hydrochloride | | mesylate | (Daiichi |
| | | | | | Sankyo Co. |
| | | | | | Ltd.) |
| Ecalevo | Neodopaston | rasagiline | benztropine | biperiden | cabergoline |
| (Sandoz) | (Daiichi Sankyo | mesylate | mesylate | hydrochloride | |
| | Co. Ltd.) | | | | |
| carbidopa | cerebrolysin | diphenhydramine | diphenhydramine | entacapone | ergoloid |
| | | | | | mesylates |
| | | | hydrochloride | | |
| ianabecestat | ibudilast | levodopa | mazaticol | nicergoline | opicapone |
| orphenadrine | oxytocin | pergolide | piroheptine | pramipexole | procyclidine |
| | | mesylate | | dihydrochloride | hydrochloride |
| profenamine | rasagiline | risperidone | ropinirole | safinamide | scopolamine |
| | | | hydrochloride | mesylate | |
| scopolamine | selegiline | talipexole | taurine | tertomotide | tetrabenazine |
| hydrobromide | hydrochloride | | | | |
| tiapride | tolcapone | trihexyphenidyl | zonisamide | cycrimine | tacrine |
| | | | | | hydrochloride |
| lemborexant | ABBV-951 | acetylleucine | ASD-005 | ASD-006 (Asdera) | azeliragon |
| | (Abb Vie) | | (Asdera) | | |
| cromolyn in | dexamethasone | E-2027 | F-627 | omaveloxolone | RG-6042 |
| combination | sodium | (Eisai) | (Generon | | (Chugai) |
| with | phosphate | | (Shanghai))) | | |
| ibuprofen | | | | | |
| RT-001 | cyclobenzaprine | Trappsol | tricaprilin | troriluzole | valproate |
| (Retrotope | hydrochloride | Cyclo | | hydrochloride | sodium |
| Inc) | | | | | |
| vatiquinone | venglustat | verdiperstat | aplindore | betamethasone | dexmedetomidine |
| | malate | | fumarate | | |
| dextro | laquinimod | masupirdine | mesdopetam | montelukast sodium | neflamapimod |
| epicatechin | sodium | | | | |
| vafidemstat | alpha- | Bisnorcymserine | dimethyl | lithium citrate | nabiximols |
| | dihydrotetrabenazine | | fumarate | | |
| gemfibrozil in | davunetide | gemfibrozil | hydralazine | idalopirdine | lithium |
| combination | | | hydrochloride | | salicylate |
| of tretinoin | | | | | |
| E-2012 (Eisai) | ELND-005 | begacestat | FK-962 | GSI-136 (Pfizer) | S-8510 |
| | | | (Astellas) | | (Shionogi) |
| TAK-065 | ABT-099 | and | | | |
| (Takeda) | (AbbVie) | tolfenamic | | | |
| | | acid. | | | |

In the case of AD, example small molecules that inhibit aggregation include apomorphine and carbenoxolone.

A small molecule neurological therapeutic agent may exert the therapeutic effects through various mechanisms. For example, the small molecule neurological agent may reduce mitochondrial dysfunction and ROS production, protein oxidation, lipid peroxidation, nitrosative stress, protein aggregation, amyloidopathy, tauopathy, DNA damage, depletion of endogenous antioxidant enzymes, proteosomeal dysfunction, microglial activation, neuroflammation, and/or neuroepigenetic modification.

A small molecule neurological therapeutic agent may include β2-adrenergic agonists, c-Abl inhibitors, cholinesterase inhibitors, leucine-rich repeat kinase 2 inhibitors, glucocerebrosidase inhibitors, glycogen synthase kinase 3β inhibitors, N-acetylglucosaminidase inhibitors, O-GlcNAcase inhibitors, or anti-inflammatory compounds.

In some embodiments, the neurological therapeutic agent comprises a protein or peptide. In some embodiments, the neurological therapeutic agent comprises a fusion protein. In certain embodiments, the peptide or protein is a chaperone protein or a co-chaperone that facilitates the proper folding of a target peptide or protein. Chaperone proteins assist the conformational folding or unfolding and the assembly or disassembly of other proteins. Exemplary chaperone proteins include, but are not limited to, heat shock proteins (e.g., Hsp104, Hsp90, Hsp70, Hsp27), αB-crystallin, clusterin, α2-macroglobulin, haptoglobin, human tetrameric transthyretin, proSAAS, protein 7B2, ERdj3/DNAJB11, GRP78/BiP, GRP94, GRP170, calnexin, calreticulin, and protein disulfide isomerase. Co-chaperones are proteins that assist chaperones in protein folding and other functions. Exemplary co-chaperones include, but are not limited to J-proteins, DnaJ, Hsp40, DNAJC5, auxilin, RME-8, and Aha1.

In some embodiments, the neurological therapeutic agent comprises a vaccine. As used herein, a vaccine is a composition that provides active acquired immunity to a particular disease, such as a neurodegenerative disease, e.g., Alzheimer's disease. A vaccine typically contains a protein or a peptide that may be disease specific (expressed exclusively by the diseased cell) or disease associated (expressed preferentially by the diseased cell). A vaccine can typically include an adjuvant. The vaccine stimulates the body's immune system to recognize the target and to eliminate or reduce the effect of the target. For example, a vaccine may be directed to amyloid-beta and stimulate the immune system to generate active acquired immunity, e.g., specific antibodies or T cells that recognize amyloid-beta and reduce or eliminate the formation of amyloid plaque. Vaccines can be prophylactic or therapeutic. A vaccine may also be a nucleic acid encoding a protein or a peptide or extract from diseased cells. Exemplary vaccines used in neurodegenerative diseases include, but are not limited to AN-1792. A vaccine may be a nucleic acid vaccine, e.g., DNA vaccine or RNA vaccine.

In certain embodiments, the neurological therapeutic agent comprises a nucleic acid or polynucleotide. The nucleic acids or polynucleotides of the invention may include deoxynucleotides, ribonucleotides, modified deoxynucleotides, modified ribonucleotides (e.g., chemical modifications, such as modifications that alter the backbone linkages, sugar molecules, and/or nucleic acid bases), and artificial nucleic acids. In some embodiments, the polynucleotide includes, but is not limited to, oligonucleotides, genomic DNA, cDNA, peptide nucleic acids (PNA) or peptide oligonucleotide conjugates, locked nucleic acids (LNA), bridged nucleic acids (BNA), polyamides, triplex forming oligonucleotides, modified DNA, antisense DNA oligonucleotides, tRNA, mPvNA, rPvNA, modified RNA, miRNA, gRNA, and siRNA or other RNA or DNA molecules.

In certain embodiments, the polynucleotide of the invention comprises a sequence that encodes a peptide or a protein, e.g., chaperone protein, antibody, or a protein or peptide used in a vaccine. The polynucleotide can encode any of the polypeptide therapeutic agents disclosed herein.

In some embodiment, the nucleic acid or polynucleotide is an inhibitory polynucleotide that inhibits the expression of a gene, e.g., the APP gene that encodes amyloid p protein, or the MAPT gene that encodes the Tau protein. The inhibitory polynucleotide may be RNAi, shRNA, siRNA, or antisense RNA.

In certain embodiments, the polynucleotide of the invention comprises a sequence that encodes a peptide or a protein, e.g., a flow modulator, such as VEGF-c, or a neurological therapeutic agent disclosed herein, such as an antibody. The polynucleotide of the invention may be comprised in an expression vector, e.g., viral or non-viral, as described herein. In some embodiments, the polynucleotide comprises an mRNA, e.g., encoding a flow modulator, such as VEGF-c or a neurological therapeutic agent, such as an antibody, as described herein. The pharmaceutical compositions comprising polynucleotides may be administered by any of the administration methods described herein.

The route and site of administration may be chosen to enhance delivery or targeting of the nucleic acid of the invention to a particular location. For example, to target brain cells, intravenous, i.c.v, or ICM injection may be used. In certain embodiments, the neurological therapeutic agent comprises a cell. A variety of cells, e.g., immune cells (such as T cell specifically targeting amyloid plaque, B cell producing antibody specifically binding amyloid, T_{neg} cell reducing inflammatory reaction), may be used as a neurological therapeutic agent, including, fresh samples derived from subjects, primary cultured cells, immortalized cells, cell-lines, hybridomas, etc. The cells to be used as a neurological therapeutic agent may also include stem cells, such as embryonic stem cells, induced pluripotent stem cells, mobilized peripheral blood stem cells. The cells may be used for various therapeutic applications.

In some embodiments, the cell may be genetically engineered to express a neurological therapeutic agent, e.g., a protein, a peptide, an antibody, or an inhibitory RNA of the invention.

In one embodiment, the neurological therapeutic agent comprises an oligonucleotide, e.g., an antisense oligonucleotide, which can be used for the treatment of Huntington's disease.

In some embodiments, the neurological therapeutic agent comprises an antibody or an antigen binding fragment thereof. In certain embodiments, the antibody or the antigen binding fragment thereof of the invention binds specifically to a peptide or a protein that forms pathological protein aggregate. Such a peptide or protein includes, but is not limited to amyloid precursor protein, amyloid beta, fibrin, tau, apolipoprotein E (ApoE), alpha-synuclein, TDP43, and huntingtin. For example, the neurological therapeutic agent can comprise, consist essentially of, or consist of an antibody selected from the group consisting of bapineuzumab, gantenerumab, aducanumab, solanezumab, crenezumab, pepinemab, ozanezumab, lecanemab, ABT-099, AT-1501, BIIB054, and PRX002. For example, the neurological therapeutic agent can comprise, consist essentially of, or consist of an antibody or an antigen binding fragment thereof that binds specifically to a protein associated with AD, for example an antibody (such as a human or humanized antibody) that binds specifically to amyloid beta, for example, bapineuzumab, gantenerumab, aducanumab, solanezumab, BAN2401, semorinemab, zagotenemab, and/or crenezumab. Solanezumab and crenezumab bind to a helix-beta coil epitope in the midsection of amyloid beta, while bapineuzumab, gantenerumab, and aducanumab bind to the N-terminal region of amyloid beta. It will be appreciated that in methods, compositions, and uses as described herein, when a flow modulator and a neurological therapeutic agent are both administered to the subject, unless stated otherwise, the flow modulator will be understood to be a different molecule than the neurological therapeutic agent. In some embodiments, the neurological therapeutic agent comprises, consists essentially of, or consist of an antibody or binding fragment selected from the group consisting of bapineuzumab, gantenerumab, aducanumab, solanezumab, and crenezumab.

In some embodiments, if the target protein is Tau protein, exemplary antibodies include, but are not limited to, ABBV-8E12 (AbbVie), Gosuranemab (BIIB092, IPN007, Bristol-Myers Squibb), PHF1, MC1, DA31, 4E6G7, 6B2G12, TOMA, PHF6, PHF13, HJ9.3, HJ9.4, HJ8.5, 43D, 77E9, AT8, MAb86, pS404 mAb IgG2, pS409-tau, Armanezumab, PHF1, Ta9, Ta4, Ta1505, and DC8E8 (see, Jadhav et al., A walk through tau therapeutic strategies, Acta Neuropathologica Communications, 7:22, 1-31 (2019)). In some other embodiments, if the target protein is Tau protein, a vaccine (peptide) may be used as the neurological therapeutic agent. Exemplary vaccines include, but are not limited to, Tau 379-408, Tau 417-426, Tau 393-408, Tau 379-408, Tau 195-213, Tau 207-220, Tau 224-238, Tau aa 395-406, Human paired helical filaments (PHF's) Tau 229-237, Tau 199-208, Tau 209-217, Tau 294-305, and Tau 379-408 (see, Jadhav et al, supra).

In certain embodiments, if the target protein is fibrin, exemplary antibodies include, but are not limited to, 5B8 as described in Ryu et al., Fibrin-targeting immunotherapy protects against neuroinfimmation and neurodegeneration, Nature Immunology 19, 1212-1223 (2018).

In some embodiments, if the target protein is apolipoprotein E (ApoE), exemplary antibodies include, but are not limited to HAE4 as described in Liao et al., Targeting of nonlipidated, aggregated apoE with antibodies inhibits amyloid accumulation, J. of Clin. Invest., 128(5): 2144-2155.

In some embodiments, for example, if the proteinopathy is Huntington's disease, the antibody may be an antibody that binds specifically to semaphorin-4D, for example pepinemab, or an antibody that binds specifically to huntingtin, for example, the antibodies disclosed in US20170166631. In some embodiments, for example, if the proteinopathy is ALS, the antibody binds specifically to neurite outgrowth inhibitor A (e.g., ozanezumab) or to CD40L (e.g., AT-1501 (Anelixis)). In some embodiments, for example, if the proteinopathy is Parkinson's disease, the neurological therapeutic agent may be an antibody binds specifically to alpha-synuclein. Exemplary anti-alpha-synuclein antibodies include, but are not limited to, BIIB054 (Biogen), PRX002/RG7935 (Roche), prasinezumab (Roche), PD-1601 (AbbVie), 1H7, 5C1, A1-A6, 9E4, 274, NbSyn87*PEST, NAC32, NAC1, AC14, VH14*PEST, syn303, AB1, Human single-chain Fv D10, D5, syn-O1, syn-02, syn-O4, mAb47, syn-10H, syn-F1, syn-F2, LS4-2G12 (see, Wang, et al., Progress of immunotherapy of anti-α-synuclein in Parkinson's disease, Biomedicine & Pharmacotherapy, 115: 108843 (2019)). In some embodiments, for example, if the proteinopathy is Parkinson's disease, the antibody may be cinpanemab, ABBV-0805 (AbbVie). In some embodiments, the neurological therapeutic agent comprises, consists essentially of, or consists of an antibody or binding fragment selected from the group consisting of bapineuzumab, gantenerumab, aducanumab, solanezumab, and crenezumab, pepinemab, ozanezumab, AT-1501, BIIB054, and PRX002.

In certain embodiments, for example, if the target protein is TDP43, exemplary antibodies include, but are not limited to, the antibodies disclosed in U.S. Pat. Nos. 10,202,443, 8,940,872, or Patent Publication Nos. WO2018218352, WO2019134981, (incorporated herein by reference), antibody 3B12A (disclosed in Scientific Reports, 8:6030 (2018), DOI:10.1038/s41598-018-24463-3).

In some embodiments, the antibody of the present invention includes bispecific antibodies of multispecific antibodies. Bispecific antibodies or multispecific antibodies recognize more two or more epitopes. The two or more epitopes may be located on a same protein or on different proteins. Exemplary bispecific or multispecific antibodies include, but are not limited to bispecific monoclonal antibodies to inhibit BACE1 and MAPT for Alzheimer's Disease developed by Denali Therapeutic Inc.

The base structure of an antibody is a tetramer, which includes two heavy chains and two light chains. Each chain comprises a constant region, and a variable region. Generally, the variable region is responsible for binding specificity of the antibody. In a typical antibody, each variable region comprises three complementarity determining regions (CDRs) flanked by four framework regions. As such, a typical antibody variable region has six CDRs (three heavy chain CDRs, three light chain CDRs), some or all of which are generally involved in binding interactions by the antibody. The CDRs can be numbered according to an art-recognized method, for example the methodology of Kabat (Kabat, et al. in “Sequences of Proteins of Immunological Interest” Public Health Service, NIH, Washington D.C. (1987)), Chothia (Chothia and Lesk, J. Mol. Biol., 196, 901-917 (1987)), AbM (Martin et al., Proc. Natl. Acad. Sci. USA, 86:9268-9272 (1989)), contact definition (MacCallum et al., J. Mol. Biol., 262: 732-745 (1996)), or IMGT (Lefranc, “Unique database numbering system for immunogenetic analysis”, Immunology Today, 18: 509 (1997), LIGM:194). An antibody that “specifically” binds (or binds “specifically”) to an antigen (for example amyloid beta) has its ordinary and customary meaning as would be understood by one of ordinary skill in the art in view of this disclosure. It refers to the antibody preferentially binding to the antigen compared to one or more other substances. By way of example, an antibody that specifically binds to amyloid beta of some embodiments binds to amyloid beta with a numerically lower dissociation constant (K_{D}) than to other substances present in the CNS. In some embodiments, an antibody that specifically binds to amyloid beta binds with a K_{D} that is less than or equal to 1000 nM, 500 nM, 200 nM, 100 nM, 75 nM, 50 nM, 25 nM, 20 nM, 15 nM, 10 nM, 5 nM, 2 nM, 1 nM, 500 pM, 100 pM, 75 pM, 50 pM, 25 pM, 10 pm, or 5 pM, including ranges between any two of the listed values. A K_{D} for a particular antigen (for example, amyloid beta) can be determined, for example, by surface plasmon resonance, for example using a BIACORE apparatus.

A neurological therapeutic agent as in accordance with methods, compositions, and uses of embodiments herein can comprise, consist essentially of, or consist of any of a number of antibodies. In some embodiments, a neurological therapeutic agent comprises, consists essentially of, or consists of an antibody or antigen binding fragment that comprises a heavy chain variable region and a light chain variable region of any one of bapineuzumab, gantenerumab, aducanumab, solanezumab, or crenezumab. In some embodiments, a neurological therapeutic agent comprises, consists essentially of, or consists of an antibody or antigen binding fragment that comprises a heavy chain variable region and a light chain variable region that are, respectively, at least 80%, 85%, 90%, 95%, 96%, 97%, 98%, or 99% identical to a heavy chain variable region and light chain variable of bapineuzumab, gantenerumab, aducanumab, solanezumab, or crenezumab. In some embodiments, a neurological therapeutic agent comprises, consists essentially of, or consists of an antibody or antigen binding fragment that comprises a light chain variable region that is at least 80%, 85%, 90%, 95%, 96%, 97%, 98%, or 99% identical to a light chain variable region of bapineuzumab, gantenerumab, aducanumab, solanezumab, or crenezumab. The antibody or antigen binding fragment can further comprise a heavy chain variable region of the noted antibody (bapineuzumab, gantenerumab, aducanumab, solanezumab, or crenezumab). In some embodiments, a neurological therapeutic agent comprises, consists essentially of, or consists of an antibody or antigen binding fragment that comprises a HCDR1, a HCDR2, and a HCDR3 of a heavy chain variable region, and a LCDR1, a LCDR2, and a LCDR3 of a light chain variable region of any one of bapineuzumab, gantenerumab, aducanumab, solanezumab, or crenezumab (that is, the antibody or antigen binding fragment comprises the noted six CDR's of any one of the listed antibodies). In some embodiments, the neurological therapeutic agent comprises, consists essentially of, or consist of an antibody selected from the group consisting of bapineuzumab, gantenerumab, aducanumab, solanezumab, and crenezumab.

The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody will typically comprise at least one variable domain. The variable domain may be of any size or amino acid composition and will generally comprise at least one CDR which is adjacent to or in frame with one or more framework sequences. In antigen-binding fragments having a VH domain associated with a VL domain, the VH and VL domains may be situated relative to one another in any suitable arrangement. For example, the variable region may be dimeric and contain VH-VH, VH-VL or VL-VL dimers. Alternatively, the antigen-binding fragment of an antibody may contain a monomeric VH or VL domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present invention include: (i) VH-CH1; (ii) VH-CH2; (iii) VH-CH3; (iv) VH-CH1-CH2; (v) VH-CH1-CH2-CH3; (vi) VH-CH2-CH3; (vii) VH-CL; (viii) VL-CH1; (ix) VL-CH2; (x) VL-CH3; (xi) VL-CH1-CH2; (xii) VL-CH1-CH2-CH3; (xiii) VL-CH2-CH3; and (xiv) VL-CL. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids which result in a flexible or semi-flexible linkage between adjacent variable and/or constant domains in a single polypeptide molecule. Moreover, an antigen-binding fragment may comprise a homo-dimer or hetero-dimer (or other multimer) of any of the variable and constant domain configurations listed above in non-covalent association with one another and/or with one or more monomeric VH or VL domain (e.g., by disulfide bond(s)).

An antibody, in accordance with methods, uses, compositions, and pharmaceutical compositions of some embodiments herein, includes an antibody of any class, such as IgD, IgE, IgG, IgA, or IgM (or sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The term “antibody” also encompasses antibody mimetics.

As with full antibody molecules, antigen-binding fragments may be monospecific or multispecific (e.g., bispecific). A multispecific antigen-binding fragment of an antibody will typically comprise at least two different variable domains, wherein each variable domain is capable of specifically binding to a separate antigen or to a different epitope on the same antigen. Any multispecific antibody format, including the exemplary bispecific antibody formats disclosed herein, may be adapted for use in the context of an antigen-binding fragment of an antibody of the present invention using routine techniques available in the art.

A number of approaches are available for producing suitable antibodies that specifically bind to a target peptide or protein, e.g., amyloid beta, α-synuclein, fibrin, tau, apolipoprotein E (ApoE), or TDP43, in accordance with methods and uses of embodiments herein. For example, in some embodiments, a host organism is immunized with an antigen comprising, consisting essentially of, or consisting of an amyloid beta, for example amyloid precursor protein (APP) or a fragment thereof. By way of example, a sequence of APP is available as Uniprot accession no. P56199 (SEQ ID NO: 5 MLPGLALLLLAAWTARALEVPTDGNAGLLAEPQIAMFCGRLNMHMNVQNGKWDSDPSGT KTCIDTKEGILQYCQEVYPELQITNVVEANQPVTIQNWCKRGRKQCKTHPHFVIPYRCLVGEF VSDALLVPDKCKFLHQERMDVCETHLHWHTVAKETCSEKSTNLHDYGMLLPCGIDKFRGVE FVCCPLAEESDNVDSADAEEDDSDVWWGGADTDYADGSEDKVVEVAEEEEVAEVEEEEAD DDEDDEDGDEVEEEAEEPYEEATERTTSIATTTTTTTESVEEVVREVCSEQAETGPCRAMISR WYFDVTEGKCAPFFYGGCGGNRNNFDTEEYCMAVCGSAMSQSLLKTTQEPLARDPVKLPTT AASTPDAVDKYLETPGDENEHAHFQKAKERLEAKHRERMSQVMREWEEAERQAKNLPKAD KKAVIQHFQEKVESLEQEAANERQQLVETHMARVEAMLNDRRRLALENYITALQAVPPRPR HVFNMLKKYVRAEQKDRQHTLKHFEHVRMVDPKKAAQIRSQVMTHLRVIYERMNQSLSLL YNVPAVAEEIQDEVDELLQKEQNYSDDVLANMISEPRISYGNDALMPSLTETKTTVELLPVN GEFSLDDLQPWHSFGADSVPANTENEVEPVDARPAADRGLTTRPGSGLTNIKTEEISEVKMD AEFRHDSGYEVHHQKLVFFAEDVGSNKGAIIGLMVGGVVIATVIVITLVMLKKKQYTSIHHG VVEVDAAVTPEERHLSKMQQNGYENPTYKFFEQMQN). By way of example, a polypeptide comprising, consisting essentially of, or consisting of the amino acid sequence of SEQ ID NO: 5 sequence can be used to immunize a host in order to produce antibodies that bind specifically to amyloid beta in accordance with some embodiments. The host organism can be a non-human mammal such as a mouse, rat, guinea pig, rabbit, donkey, goat, or sheep. Isolated antibody-producing cells can be obtained from the host organism, and the cells (or antibody-encoding nucleic acids thereof) can be screened for antibodies that binds specifically to amyloid beta. In some embodiments, antibody-producing cells are immortalized using hybridoma technology, and the resultant hybridomas are screened for antibodies that bind specifically to amyloid beta. In some embodiments, antibody-encoding nucleic acids are isolated from antibody-producing cells, and screened for antibodies that bind specifically to amyloid beta. An example protocol for screening human B cell nucleic acids is described in Huse et al., Science 246:1275-1281 (1989), which is hereby incorporated by reference in its entirety. In some embodiments, nucleic acids of interest are identified using phage display technology (See, e.g., Dower et al., WO 91/17271 and McCafferty et al., WO 92/01047, each of which is hereby incorporated by reference in its entirety). Phage display technology can also be used to mutagenize variable regions (or portions thereof such as CDRs) of antibodies previously shown to have affinity for amyloid beta. Variant antibodies can then be screened by phage display for antibodies having desired affinity to amyloid beta.

Unless stated otherwise, wherever an “antibody” is mentioned herein, a binding fragment of that antibody is also contemplated. In some embodiments, the antibody that specifically binds to a target peptide or protein, e.g., amyloid beta, is formatted as an antigen binding fragment (which may be referred to herein simply as a “binding fragment”). Example antigen binding fragments suitable for methods and uses of some embodiments can comprise, consist essentially of, or consist of a construct selected from the group consisting of Fab, Fab′, Fab′-SH, F(ab′)₂, and Fv fragments; Fd fragment; minimal recognition units consisting of the amino acid residues that mimic the hypervariable region of an antibody (e.g., an isolated complementarity determining region (CDR) such as a CDR3 peptide), or a constrained FR3-CDR3-FR4 peptide; minibodies; diabodies; and single-chain fragments such as single-chain Fv (scFv) molecules. Bispecific or multispecific antibodies or antigen binding fragments are also contemplated in accordance with methods and uses of some embodiments. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment,” as used herein. In some embodiments, the amyloid beta antibody is chimeric, human, or humanized. In some embodiments, the amyloid beta antibody is human, or humanized.

In some embodiments, for example if human monoclonal antibodies are of interest, the host comprises genetic modifications to produce or facilitate the production of human immunoglobulins. For example, XenoMouse™ mice were engineered with fragments of the human heavy chain locus and kappa light chain locus, respectively, which contained core variable and constant region sequences (described in detail Green et al. Nature Genetics 7:13-21 (1994), which is hereby incorporated by reference in its entirety). For example, mice have been engineered to produce antibodies comprising a human variable regions and mouse constant regions. The human heavy chain and light chain variable regions can then be reformatted onto a human constant region to provide a fully human antibody (described in detail in U.S. Pat. No. 6,787,637, which is hereby incorporated by reference in its entirety), For example, in a “minilocus” approach, an exogenous Ig locus is mimicked through the inclusion of pieces (individual genes) from the Ig locus. Thus, one or more VH genes, one or more DH genes, one or more JH genes, a mu constant region, and a second constant region (preferably a gamma constant region) are formed into a construct for insertion into an animal such as a mouse (See, e.g., U.S. Pat. No. 5,545,807, which is hereby incorporated by reference in its entirety). Another approach, includes reconstituting SCID mice with human lymphatic cells, e.g., B and/or T cells. The mice are then immunized with an antigen and can generate an immune response against the antigen (See, e.g., U.S. Pat. No. 5,476,996, which is hereby incorporated by reference in its entirety).

In some embodiments, a host monoclonal antibody is formatted as a chimeric antibody or is humanized, so that the antibody comprises at least some human sequences. By way of example, an approach for producing humanized antibodies can comprise CDR grafting. For example, an antigen can be delivered to a non-human host (for example a mouse), so that the host produces antibody against the antigen. In some embodiments, monoclonal antibody is generated using hybridoma technology. In some embodiments, V gene utilization in a single antibody producing cell of the host is determined. The CDR's of the host antibody can be grafted onto a human framework. The V genes utilized in the non-human antibody can be compared to a database of human V genes, and the human V genes with the highest homology can be selected, and incorporated into a human variable region framework. See, e.g., Queen, U.S. Pat. No. 5,585,089, which is hereby incorporated by reference in its entirety.

Isolated oligonucleotides encoding an antibody of interest can be expressed in an expression system, such as a cellular expression system or a cell-free system in order to produce an antibody that binds specifically to amyloid beta in accordance with methods and uses of embodiments herein. Exemplary cellular expression systems include yeast (e.g., mammalian cells such as CHO cells or BHK cells, E. coli, insect cells, Saccharomyces, Pichia) transformed with recombinant yeast expression vectors containing the nucleotide sequences encoding antibodies; insect cell systems infected with recombinant virus expression vectors (e.g., baculovirus) containing sequences encoding antibodies; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing nucleotide sequences encoding antibodies; mammalian cell systems (e.g., COS, CHO, BHK, 293, 3T3) harboring recombinant expression constructs containing promoters derived from the genome of mammalian cells (e.g., metallothionein promoter) or from mammalian viruses.

In some embodiments, the neurological therapeutic agent comprises a chimeric antigen receptor T-cell (CAR-T cell) that binds specifically to a protein associated with the proteinopathy of the patient. In some embodiments, for example if the neurological disease or disorder comprises a tauopathy and/or an amyloidosis such as AD, the neurological therapeutic agent comprises a chimeric antigen receptor T-cell (CAR-T cell) that binds specifically to an amyloid beta protein. For example, the CAR-T cell can comprise a chimeric antigen receptor comprising a heavy chain variable region and a light chain variable region of any of the antibodies described herein. For example, the CAR-T cell can comprise a chimeric antigen receptor comprising a HCDR1, HCDR2, HCDR3, LCDR1, LCDR2, and LCDR3 of any of the antibodies described herein.

In certain embodiments, a neurological therapeutic agent of the invention may be used in combination with one or more different neurological therapeutic agents of the invention. For example, a small molecule neurological therapeutic agent may be used in combination with an antibody therapeutic agent. Different neurological therapeutic agents may be formulated in a same pharmaceutical composition or in different pharmaceutical compositions. Different neurological therapeutic agents may be administered concurrently or sequentially.

Neurological Diseases

Methods, uses, and compositions in accordance with some embodiments herein can be useful for diagnosing, treating, preventing, inhibiting, ameliorating, or reducing the symptoms of one or more neurological diseases, or compositions for use in these methods. In some embodiments, the neurological disease is a proteinopathy. In some embodiments, the neurological disease comprises a proteinopathy as described herein (e.g., a tauopathy and/or amyloidosis such as AD). Such diseases can occur in subjects, for example humans, as well as non-human animals, such as non-human mammals, and non-human primates.

In some embodiments, a neurological disease such as a neurodegenerative, neurodevelopmental, neuroinflammatory, or neuropsychiatric disease associated with accumulation of macromolecules, cells, and debris in the CNS is treated, prevented, inhibited, or reduced by methods, uses, or compositions that increase flow, drainage, and/or clearance in meningeal lymphatic vessels. In some embodiments, neurodegenerative, neurodevelopmental, neuroinflammatory, or neuropsychiatric diseases associated with accumulation of macromolecules, cells, and debris in the CNS are treated, prevented, inhibited, or reduced by methods, uses, or compositions that counteract the effects (e.g., changes in the hippocampal transcriptome) of decreased flow with or without restoring flow. In some embodiments, neurological diseases associated with accumulation of macromolecules, cells, and debris in the CNS are treated, prevented, inhibited, or reduced. Examples of neurological diseases include proteinopathies, for example tauopathies and/or amyloidosis such as AD (e.g., familial AD and/or sporadic AD). Examples of neurological diseases include AD (such as familial AD and/or sporadic AD), dementia, age-related dementia, PD, cerebral edema, ALS, PANDAS, meningitis, hemorrhagic stroke, ASD, epilepsy, Down's syndrome, HCHWA-D, Familial Danish/British dementia, DLB, LB variant of AD, MSA, FENIB, FTD, HD, Kennedy disease/SBMA, DRPLA; SCA type I, SCA2, SCA3 (Machado-Joseph disease), SCA6, SCA7, SCA17, CJD (such as familial CJD), Kuru, GSS, FFI, CBD, PSP, CAA, AIDS-related dementia complex, brain injury, traumatic brain injury, brain hemorrhage, subarachnoid brain hemorrhage, stroke, or a combination of two or more of the listed items. By way of example, neurological diseases can include AD, dementia, age-related dementia, PD, cerebral edema, ALS, PANDAS, meningitis, hemorrhagic stroke, ASD, and epilepsy. In some embodiments, the neurological disease comprises, consists essentially of, or consists of a proteinopathy, for example AD (such as familial AD and/or sporadic AD), Down's syndrome, HCHWA-D, Familial Danish/British dementia, PD, DLB, LB variant of AD, MSA, FENIB, ALS, FTD, HD, Kennedy disease/SBMA, DRPLA; SCA type I, SCA2, SCA3 (Machado-Joseph disease), SCA6, SCA7, SCA17, CJD (such as familial CJD), Kuru, GSS, FFI, CBD, PSP, CAA, AIDS-related dementia complex, or a combination of two or more of any of the listed items. In some embodiments, neurological diseases associated with amyloid beta for example an amyloidosis such as AD (e.g., familial AD and/or sporadic AD) in the CNS are treated, prevented, inhibited, or reduced by methods, uses, or compositions that counteract the effects of decreased flow with or without restoring flow.

In some embodiments, the neurological disease, for example a proteinopathy (such as a tauopathy and/or an amyloidosis, e.g., AD) can be prevented, treated, or ameliorated prophylactically. Accordingly, a subject having one or more risk factors for the neurological disease can be determined to be in need of receiving a method, use, or composition described herein. For example, a subject may have accumulated amyloid beta plaques in their CNS, and may benefit from increased flow, increased drainage, increased clearance and/or reduction of amyloid beta plaques, even if they do not yet have an neurological disease diagnosis based on cognitive symptoms.

A number of risk factors for AD are suitable as risk factors in accordance with methods, compositions, and uses of some embodiments herein, for example familial AD, a genetic marker for AD, or a symptom of AD such as early dementia. The foremost risk factor for sporadic AD is age. However, increased risk of this form of AD has also been attributed to diverse genetic abnormalities. One of them is diploidy for apolipoprotein-EE4 (Apo-EE4), widely viewed as a major genetic risk factor promoting both early onset of amyloid beta aggregation and defective amyloid beta clearance from the brain (Deane et al., 2008; Zlokovic, 2013). Other genetic variants that significantly increase the risk for sporadic AD are Apo-J (or clusterin), phosphatidylinositol-binding clathrin assembly protein (PICALM), complement receptor 1 (CR1), CD33 or Siglec-3, and triggering receptor expressed on myeloid cells 2 (TREM2). All of these proteins, interestingly, have been implicated in different mechanisms of amyloid beta removal from the brain (Bertram et al., 2008; Guerreiro et al., 2013; Harold et al., 2009; Lambert et al., 2009, 2013; Naj et al., 2011). In some embodiments, the risk factor for AD is selected from the group consisting of at least one of the following: diploidy for apolipoprotein-E-epsilon-4 (apo-E-epsilon-4), a variant in apo-J, a variant in phosphatidylinositol-binding clathrin assembly protein (PICALM), a variant in complement receptor 1 (CR3), a variant in CD33 (Siglee-3), or a variant in triggering receptor expressed on myeloid cells 2 (TREM2), age, or a symptom of dementia.

### Methods of the Invention

The present invention features various methods in modulating the central nervous system of a subject in need thereof. The methods include, but are not limited to, treating a neurological disease in a subject, reducing the risk, or delaying the onset of a neurological disease in a subject, increasing flow in fluid in the central nervous system of a subject, reducing protein aggregates in the central nervous system of a subject, increasing clearance of molecules (such as proteins, e.g., amyloid beta) from the central nervous system of a subject, increasing clearance of cells from the central nervous system of a subject, reducing a microglia inflammatory response in the central nervous system of a subject, reducing a neurite dystrophy or reducing vascular fibrinogen deposition in the central nervous system of a subject. In some embodiments, the methods modulate the central nervous system in the brain of the subject.

In some embodiments, the methods include administering to a subject an effective amount of a neurological therapeutic agent according to the present invention. In certain embodiments, the methods include administering to the subject an effective amount of flow modulator according to the present invention. In some embodiments, the methods of the present invention include administering to a subject in need thereof an effective amount of a neurological therapeutic agent and an effective amount of flow modulator. In certain embodiments, the neurological therapeutic agent and/or the flow modulator comprises a protein or a peptide or a polynucleotide encoding the protein or the peptide.

In certain embodiments, the flow modulator and the neurological therapeutic agent are formulated in the same pharmaceutical composition. In some embodiments, the flow modulator and the neurological therapeutic agent are formulated in different pharmaceutic composition.

In some embodiments, the flow modulator and the neurological therapeutic agent are administered to the subject concurrently. In certain embodiments, the flow modulator and the neurological therapeutic agent are administered to the subject sequentially. For example, the neurological therapeutic agent may be administered to the subject prior to, concurrently with, and subsequent to the administration of the flow modulator.

In certain embodiments, the flow modulator and the neurological therapeutic agent are administered to the subject via the same administration route. In some embodiments, the flow modulator and the neurological therapeutic agent are administered to the subject via different administration route.

In some embodiments where the flow modulator and the neurological therapeutic agent are administered to a subject in need thereof, both the flow modulator and the neurological therapeutic agent comprise protein or peptide or a polynucleotide encoding the protein or the peptide. In certain embodiments, the flow modulator comprises a protein or a peptide and the neurological therapeutic agent comprises a protein or a peptide. In certain embodiments, the flow modulator comprises a protein or a peptide and the neurological therapeutic agent comprises a polynucleotide that encodes a protein or a peptide. In certain embodiments, the flow modulator comprises a polynucleotide that encodes a protein or a peptide and the neurological therapeutic agent comprises a protein or a peptide. In certain embodiments, the flow modulator comprises a polynucleotide that encodes a protein or a peptide and the neurological therapeutic agent comprises a polynucleotide that encodes a protein or a peptide. The polynucleotide, including expression vector, e.g., viral vector, is described elsewhere herein.

### Methods of Identifying Subject Having Enhanced Risk of Developing Neurological Diseases

In one aspect, the invention is based upon, at least in part, the surprising discovery that a subject has degeneration of lymphatic vasculature in the central nervous system of the subject prior to the onset of the neurological disease. The term “degeneration of lymphatic vasculature,” as used herein, refers to the reduction or loss or lymphatic vessel coverage (in area) in the central nervous system. The reduced coverage may be cause by the reduced length, the diameter, and/or branching point of lymphatic vessels. In certain embodiments, the degeneration of lymphatic vessel occurs at the superior sagittal sinus, dural confluence of sinuses, the transverse (TS), sigmoid (SS), or petrosquamosal (PSS) sinuses.

Accordingly, in some embodiments, the invention provides a method of identifying a subject that has an enhanced risk of developing neurological disease prior to the onset of the neurological disease. The term “enhanced risk,” as used herein, refers to a higher probability to develop certain neurological diseases, e.g., Alzheimer's disease, as compared to a reference probability (reference risk). The reference risk is the probability of developing such a neurological disease in general population. The method includes detecting the degeneration of lymphatic vasculature in the central nervous system of the subject. Any methods that can be used to detect the degeneration of the lymphatic vasculature in the central nervous system are encompassed in this invention. In certain embodiments, the detection method is a non-invasive detection method that visualizes the lymphatic vasculature of the central nervous system. Exemplary non-invasive detection method includes magnetic resonance imaging as described in Abstinta et al., Human and nonhuman primate meninges harbor lymphatic vessels that can be visualized noninvasively by MRI, eLife 2017: 6: e29738, DOI: https://doi.org/10.7554/eLife.29738, incorporated herein by reference. To use MRI to visualize the lymphatic vessels in the central nervous system, a magnetic dye is administered to the subject. The magnetic dye has molecules that are small enough to leak out of blood vessels in the dura into lymphatic vessels, but too big to pass through the blood-brain barrier and enter other parts of the brain. By adjusting the parameters of the MRI, the lymphatic vessels of the central nervous system can be specifically visualized.

In certain embodiments, the lymphatic vasculature can be visualized using in vivo fluorescence imaging method. Exemplary fluorescence imaging in human was described in Piper et al., Toward whole-body fluorescence imaging in humans, PLoS One, 2013; 8(12): e83749, incorporated herein by reference.

The degeneration of lymphatic vasculature may be reflected in the decrease of lymphatic coverage by about 10%, about 20%, about 30%, about 40%, about 50%, about 60%, about 70%, about 80%, about 90%, about 99%, or between about 10% to about 99%.

In one aspect, this invention is based upon, at least in part, that a subject with enhanced risk of developing a neurological disease, e.g., Alzheimer's disease, has increased number of immune cell in the central nervous system.

Accordingly, in some embodiments, the present invention provides a method of identifying a subject that has an enhanced risk of developing a neurological disease, e.g., Alzheimer's disease, prior to the onset of the neurological disease. The method includes detecting the increase in the number of immune cells in the central nervous system. The number of immune cells increases if more immune cells are identified in the central nervous system as compared to a reference number of immune cells. The reference number of immune cells is a number of immune cells in the central nervous system of a healthy subject who is known to have a reference risk or the number of immune cells in the central nervous system in a general population.

In certain embodiments, the immune cells are CD45^{high} microglia or recruited lymphocytes. In some embodiments, the immune cells are CD45^{int} microglia or recruited lymphocytes that express H-2KD. In some other embodiments, the immune cells are selected from the group consisting of B cells, CD4⁺ T cells, CD8⁺ T cells, and type 3 innate lymphoid cells (ILC3s).

In some embodiments, the number of immune cells in a subject's central nervous system may be determined by in vivo fluorescence imaging. For example, an antibody specific to a cell surface protein may be conjugated to a fluorescence entity. The antibody-fluorescence entity complex may be administered to a subject. The fluorescence density may reflect the number of the immune cells.

In some other embodiments, the number of immune cells may be increased by about 10%, about 20%, about 30%, about 40%, about 50%, about 60%, about 70%, about 80%, about 90%, about 99%, between about 10% to about 99%, about one fold, about two fold, about 4 fold, about 8 fold, about 16 fold, about 32 fold, about 50 fold, about 100 fold, or about one to about 100 fold, or more than about 100 fold.

In one aspect, the subject is a human subject. The human subject may be about 20 years old, about 30 years old, about 40 years old, about 50 years old, about 60 years old, about 70 years old, about 80 years old, about 90 years old, about 100 years old, or any age between about 20 and about 100 years old. In some embodiments, the human subject is previously known to have an enhanced risk of developing a neurologic disease, e.g., Alzheimer's disease. Such an enhanced risk may be evaluated by investigating the family history of the subject or by genetic screening.

### Methods of Reducing Risk of Developing Neurological Disease

In one aspect, the present invention is based upon, at least in part, the surprising discovery that administration of a neurological therapeutic agent, e.g., an antibody against AD, prior to the onset of a neurological disease, e.g., Alzheimer's disease, can reduce the risk of developing such neurological disease. Accordingly, the present invention provides a method of reducing the risk, or delaying the onset of a neurological disease. The method includes administering to a subject an effective amount of a neurological therapeutic agent according to the present invention. In some embodiment, the subject is identified to have an enhanced risk of developing the neurological disease according to any method disclosed herein.

In certain embodiments, the methods of reducing the risk, or delaying the onset of a neurological disease further include administering to the subject an effective amount of flow modulator according to the present invention.

In some embodiments, the methods of the present invention includes administering to a subject in need thereof an effective amount of a neurological therapeutic agent and an effective amount of flow modulator.

Methods, Compositions, and Uses for Increasing Flow

Some embodiments include methods of, compositions for use, or uses for increasing flow in fluid in the central nervous system of a subject, or compositions for use in these methods. It noted that in some embodiments, the components of any of the noted compositions can be provided separately as “product combinations” in which the components are provided in two or more precursor compositions, which can either be combined to form the final composition (e.g., mix a flow modulator with a neurological disease therapeutic agent to arrive at a final composition comprising the flow modulator neurological disease therapeutic agent), or used in conjunction to achieve an effect similar to the single composition (e.g., administer a flow modulator and neurological disease therapeutic agent to a subject simultaneously or sequentially). Some embodiments include a composition or product combination comprising a flow modulator (e.g., VEGFR3 agonist and/or FGF), and a neurological disease therapeutic agent. The neurological disease therapeutic agent can be different from the flow modulator. The composition can be for medical use, for example, for use in treating, preventing, or ameliorating the symptoms of a neurological disease, for example a proteinopathy as described herein (e.g., a tauopathy and/or amyloidosis such as AD). The methods or uses can include determining whether the subject is in need of increased fluid flow in the central nervous system. If the subject is in need of increased fluid flow, the method or use can include administering an effective amount of flow modulator (such as a VEGFR3 agonist and/or FGF2) to a meningeal space of the subject and administering a neurological therapeutic agent to the subject (for example, to the CNS, such as the meningeal space). The flow modulator (e.g., VEGFR3 agonist and/or FGF2) and neurological therapeutic agent can be administered in the same composition, or in separate compositions as described herein. Without being limited by theory, the amount of flow modulator (e.g., VEGFR3 agonist and/or FGF2) can increase flow for example, by increasing the diameter of a meningeal lymphatic vessel of the subject, by increasing the quantity of meningeal lymphatic vessels of the subject, and/or by increasing drainage through meningeal lymphatic vessels of the subject. Thus, fluid flow in the central nervous system of the subject can be increased. Further, the neurological therapeutic agent can treat, inhibit, ameliorate, reduce the symptoms of, reduce the likelihood of, or prevent the neurological disease. In some embodiments, the neurological therapeutic agent (e.g., amyloid beta antibody) synergizes with the flow modulator (e.g., VEGFR3 agonist and/or FGF2). The synergy can comprise greater clearance of protein deposits (e.g., amyloid deposits) than either the neurological therapeutic agent or flow modulator on its own, for example at least 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, or 95%, including ranges between any two of the listed values, less amyloid plaque density, and/or at least 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, or 95%, including ranges between any two of the listed values lower amyloid plaque size compared to either the neurological therapeutic agent or flow modulator alone. In some embodiments, the synergy comprises enhancement of memory, and/or delay in the onset or progression of dementia. In some embodiments, the fluid comprises cerebral spinal fluid (CSF), interstitial fluid (ISF), or both. In some embodiments, the VEGFR3 agonist comprises, consists essentially of, or consists of VEGF-c or VEGF-d or an analog, variant, or fragment thereof. It is also contemplated that for compositions and methods and uses in some embodiments herein, FGF2 can be substituted for the indicated VEGFR3 agonist in order to increase flow, or can be used in addition to a VEGFR3 agonist in order to increase flow.

Such methods of, compositions for, or use for increasing fluid flow in the CNS can be useful for treating, preventing, or ameliorating the symptoms of neurological diseases associated with the increased concentration and/or accumulation of molecules, cells. or debris in the CNS (e.g., protein deposits such as amyloid deposits), for example in a neurological disease, for example a proteinopathy such as a tauopathy and/or amyloidosis (e.g., AD). Accordingly, in some embodiments, a subject can be determined to be in need of increased fluid flow by determining whether the subject has a neurological disease or is at risk of developing a neurological disease. The disease can be associated with the increased concentrations and/or accumulation of molecules or cells or debris in the CNS, for example a proteinopathy such as a tauopathy and/or amyloidosis (e.g., AD). In some embodiments, the subject can be determined to be at risk for the disease, for example through having familial occurrence of the disease, by having one or more genetic or protein or metabolite markers associated with the disease, through advanced age, or by exhibiting symptoms of the disease, for example early dementia in the case of AD. As used herein, “advanced age” refers to an age characterized by a decrease in memory function, decrease in CSF production, substantial increases in neuronal senescence, and in the context of some embodiments, can include at least 65 years of age in a human, for example, at least 60, 65, 70, 75, 80, or 85, including ranges between any of these values. In some embodiments, determining whether the subject is in need of increased fluid flow comprises determining the subject to have a neurological disease, for example a proteinopathy such as a tauopathy and/or amyloidosis (e.g., AD). In some embodiments, determining whether the subject is in need of increased fluid flow comprises determining the subject to have a risk factor for the neurological disease associated with the increased concentration and/or accumulation of molecules or macromolecules or cells or debris in the CNS as described herein. In some embodiments, determining whether the subject is in need of increased fluid flow comprises determining the subject to have a risk factor, and also determining the subject to have the disease itself. In some embodiments, the neurological disease is Alzheimer's disease, and the risk factor is a risk factor for Alzheimer's disease as described herein. In some embodiments, the flow modulator (e.g., VEGFR3 agonist and/or FGF2) is administered to the subject after determining that the subject has a risk factor for the neurological disease (even if the subject does not necessarily have the disease itself), for example for prophylactic treatment or prevention. In some embodiments, the flow modulator (e.g., VEGFR3 agonist and/or FGF2) is administered to the subject after determining that the subject has the neurological disease.

Without being limited by theory, it is contemplated, according to several embodiments herein, that systemic administration is not required for the flow modulator (e.g., VEGFR3 agonist and/or FGF2) to effectively modulate meningeal lymphatic vessel size and drainage, or flow, and/or for the combination of the neurological therapeutic agent and flow modulator to inhibit, treat, reduce the likelihood of, delay the onset of, prevent, or ameliorate symptoms of the neurological disease. Accordingly, in some embodiments, the flow modulator (e.g., VEGFR3 agonist and/or FGF2), and/or the neurological therapeutic agent is administered selectively to the meningeal space of the subject. In the method, use, or composition of some embodiments, the flow modulator (e.g., VEGFR3 agonist and/or FGF2) is administered to the meningeal space, and the neurological therapeutic agent is administered to the subject. The neurological therapeutic agent may be administered to the meningeal space, or to a different location, for example, subcutaneously, intravenously, parenterally, orally, by inhalation, transdermally, or by rectal administration. In the method, use, or composition of some embodiments, the flow modulator (e.g., VEGFR3 agonist and/or FGF2), and/or the neurological therapeutic agent is administered to the meningeal space, but is not administered outside the CNS. In the method, use, or composition of some embodiments, the flow modulator (e.g., VEGFR3 agonist and/or FGF2), and/or the neurological therapeutic agent is administered to the meningeal space, but is not administered to the blood. In the method, use, or composition of some embodiments, the flow modulator (e.g., VEGFR3 agonist and/or FGF2), and/or the neurological therapeutic agent is administered to the subject by a route selected from the group consisting of at least one of the following: nasal administration, transcranial administration, contact with cerebral spinal fluid (CSF) of the subject, pumping into CSF of the subject, implantation into the skull or brain, contacting a thinned skull or skull portion of the subject with the VEGFR3 agonist and/or FGF2 and/or the neurological therapeutic agent, or expression in the subject of a nucleic acid encoding the VEGFR3 agonist and/or FGF2 and/or the neurological therapeutic agent, or a combination of any of the listed routes. In some embodiments, it is the VEGFR3 agonist that is administered. In the method, use, or composition of some embodiments, the VEGFR3 agonist is selected from the group consisting of at least one of the following: VEGF-c, VEGF-d, or an analog, variant, or functional fragment thereof. In the method, use, or composition of some embodiments, the neurological therapeutic agent comprises, consists essentially of, or consists of an amyloid beta antibody. In the method, use, or composition of some embodiments, the neurological therapeutic agent comprises, consists essentially of, or consists of an amyloid beta antibody.

In the method, use, or composition of some embodiments, the administration of the flow modulator (VEGFR3 agonist such as VEGF-c, and/or FGF2) and the neurological therapeutic agent (e.g., amyloid beta antibody) results in an increase in CNS fluid flow, meningeal lymphatic vessel diameter, meningeal lymphatic vessel number, meningeal lymphatic vessel drainage, or amelioration of symptoms of a neurological disease. For example, in some embodiments, the administration of the flow modulator (e.g., VEGFR3 agonist and/or FGF2) and the neurological therapeutic agent increases diameter of the meningeal lymphatic vessel is increased by at least about 5%, for example at least about 5%, 10%, 15%, 20%, 25%, 30%, 35%, 40%, 45% or 50%, including ranges between any two of the listed values. In some embodiments, an average diameter of a population of meningeal lymphatic vessels of the subject is increased by a value noted herein. In some embodiments, the administration of the flow modulator (e.g., VEGFR3 agonist and/or FGF2) and the neurological therapeutic agent increases fluid flow in the central nervous system of the subject, comprising increasing a rate of perfusion of fluid throughout an area of the subject's brain. In some embodiments, for example if the subject has AD, the administration of the flow modulator (VEGFR3 agonist such as VEGF-c, and/or FGF2), and neurological therapeutic agent (e.g., amyloid beta antibody) increases the ISF flow and reduces the quantity and/or average size of amyloid beta plaques in the subject's CNS. For example, the quantity of accumulated amyloid beta plaques can be reduced by at least 2%, for example, at least 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, or 95%, including ranges between any two of the listed values. For example, the average size of accumulated amyloid beta plaques can be reduced by at least 2%, for example, at least 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, or 95%, including ranges between any two of the listed values. It is shown herein that some brains of humans with AD have structures resembling amyloid beta plaques in the meninges (see US Patent Publication US20190269758A1, the contents of which are incorporated herein by reference, at Example 7). Accordingly, in some embodiments, at least some of the accumulated amyloid beta plaques are in the meninges of the subject's brain. In some embodiments, administering the combination of the flow modulator (VEGFR3 agonist such as VEGF-c, and/or FGF2), and the neurological therapeutic agent (e.g., amyloid beta antibody) increases clearance of soluble molecules in the brain of the subject. Clearance of soluble molecules can be ascertained, for example, by monitoring the retention of a particular compound, molecule, or label over an area of the brain over a particular period of time. In some embodiments, administering the combination of the FGF2 or VEFR3 agonist (e.g., VEGF-c) and the neurological therapeutic agent (e.g., amyloid beta antibody) increases clearance of soluble molecules in the brain of the subject by at least 2%, for example, at least 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, or 95%, including ranges between any two of the listed values.

Methods for assessing the fluid flow are known in the art. In some embodiments, the fluid flow can be assessed using an in vivo imaging method known in the art, such as in vivo stereomicroscope as described in Examples 8 and 10 of the present disclosure. In certain embodiments, the fluid flow can be measured by the increase of certain molecules, e.g., a protein, in the deep cervical lymph nodes, or the reduction of the molecule in the brain, or both. The method to detect and quantify a molecule, e.g., protein is known in the art. For example, the level of the molecule can be detected on a biopsy sample from the subject. In some embodiments, an imaging probe that is specific for a molecule, such as an antibody specific for such molecule conjugated with magnetic or fluorescent nanostructure, can be administered to the target tissue, e.g., brain. The image probe can be detected in vivo using methods known in the art, such as molecular magnetic resonance imaging as described in Viola et al., Towards non-invasive diagnostic imaging of early-stage Alzheimer's disease, Nature Nanotechnology, 10, 91-98 (2015).

Methods, Compositions, and Uses for Reducing Protein Aggregates

Some embodiments include methods, compositions for use, and uses for reducing a quantity of protein aggregates, such as amyloid beta, fibrin, tau, or alpha-synuclein aggregates. In one embodiment, the protein aggregate comprises amyloid beta plaques. Accordingly, the present invention provides compositions and methods for reducing accumulated amyloid beta plaques, or decreasing the rate of accumulation of amyloid beta plaques, in a subject having a neurological disease or a risk factor for such a disease, or compositions for use in such methods.

The methods or uses can include determining the subject to have the neurological disease or the risk factor. The methods or uses can include administering a flow modulator (e.g., VEGFR3 agonist and/or FGF2) to a meningeal space of the subject, so that fluid flow (e.g., flow of ISF, CSF, or both) in the central nervous system of the subject is increased, and further administering and a neurological therapeutic agent to the subject (the neurological therapeutic agent can be administered to a meningeal space or to a different location). Through increased fluid flow, the quantity of accumulated amyloid beta plaques in the subject can be reduced, or the rate of accumulation can be reduced. By way of example, the VEGFR3 agonist can comprise (or consist essentially of, or consist of) VEGF-c, and the neurological therapeutic agent can comprise (or consist essentially of, or consist of) an amyloid beta antibody. In some embodiments, at least some of the accumulated amyloid beta plaques are in the meninges of the subject's brain. In some embodiments, the quantity of accumulated amyloid beta plaques, the average size of the accumulated amyloid beta plaques, and/or the rate of accumulation, is reduced by at least 2%, for example, at least 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 100% including ranges between any two of the listed values. In some embodiments, the flow modulator (e.g., VEGFR3 agonist and/or FGF2) and/or the neurological therapeutic agent is administered selectively to the meningeal space. In some embodiments, the flow modulator (e.g., VEGFR3 agonist and/or FGF2) and/or the neurological therapeutic agent is administered to the CNS, but not outside the CNS. In some embodiments, the flow modulator (e.g., VEGFR3 agonist and/or FGF2) and/or the neurological therapeutic agent is administered to the CNS, but not blood. In some embodiments, the VEGFR3 agonist is selected from the group consisting of at least one of the following: VEGF-c, VEGF-d, or an analog, variant, or functional fragment thereof. In some embodiments, the neurological therapeutic agent comprises, consists essentially of, or consists of an amyloid beta antibody.

In some embodiments, administering the flow modulator (e.g., VEGFR3 agonist and/or FGF2) and/or the neurological therapeutic agent increases the diameter of a meningeal lymphatic vessel of the subject's brain by at least 2%, for example at least 2%, 3%, 4%, 5%, 6%, 7%, 8%, 9%, 10%, 15%, 20%, 25%, 30%, 35%, 40%, 45%, or 50%, including ranges between any two of the listed values, thus increasing flow in ISF. As noted herein, increased fluid flow in the central nervous system of the subject comprises an increased rate of perfusion of fluid throughout an area of the subject's brain. For example, increased fluid flow in the central nervous system of the subject can comprise an increased rate of perfusion out of the subject's central nervous system.

In some embodiments, the subject is known to have the neurological disease, for example AD (such as familial AD and/or sporadic AD), Down's syndrome, HCHWA-D, Familial Danish/British dementia, PD, DLB, LB variant of AD, MSA, FENIB, ALS, FTD, HD, Kennedy disease/SBMA, DRPLA; SCA type I, SCA2, SCA3 (Machado-Joseph disease), SCA6, SCA7, SCA17, CJD (such as familial CJD), Kuru, GSS, FFI, CBD, PSP, CAA, or a combination of two or more of any of the listed items. By way of example, the neurological disease can comprise a proteinopathy. In some embodiments, the method further includes determining that the subject has the neurological disease. In some embodiments, for example if the method or use is prophylactic, the method comprises determining whether the subject has the risk factor for the neurological disease, even if the subject does not necessarily have a diagnosis for the disease itself. For example, risk factors for AD that are useful in accordance with methods, compositions, and uses of some embodiments herein include diploidy for apolipoprotein-E-epsilon-4 (apo-E-epsilon-4), a variant in apo-J, a variant in phosphatidylinositol-binding clathrin assembly protein (PICALM), a variant in complement receptor 1 (CR3), a variant in CD33 (Siglee-3), or a variant in triggering receptor expressed on myeloid cells 2 (TREM2), familial AD, advanced age, or a symptom of dementia.

In certain embodiments, the present invention provides methods of reducing extracellular protein aggregates, e.g., amyloid plaque, or protein aggregates released by a cell, e.g., a neuron. For example, a neurological therapeutic agent, such as an antibody or an anti-aggregation small molecule compound, may binds or interacts with an amyloid plaque to reduce the protein aggregation.

In some embodiments, the present invention provides methods of reducing intracellular protein aggregates, e.g., a huntingtin aggregate within a cell. Through increased fluid flow by a flow modulator of the invention, e.g., VEGF-c, a neurological therapeutic agent may be delivered into a cell to reduce the formation of the protein aggregate. For example, a small inhibitory RNA may be delivered to a cell to reduce the expression of a protein, e.g., amyloid-beta, thereby reducing the formation of amyloid aggregate in the cell.

Methods for detecting protein aggregate are known in the art. For example, an imaging probe, which specifically binds to the protein aggregate, may be injected to a subject. In vivo imaging technology may be used to detect the protein aggregate. The efficacy of a treatment may be evaluated by assessing the reduction of the protein aggregate imaging signal. Such imaging methods have been described in, for example, Viola et al., Towards non-invasive diagnostic imaging of early-stage Alzheimer's disease, Nature Nanotechnology, 10, 91-98 (2015).

Methods, Compositions, and Uses of Increasing Clearance of Molecules from the CNS

Some embodiments include a method, use, or composition for use in increasing clearance of molecules (such as proteins, e.g., amyloid beta) from the central nervous system of a subject. The method or use can comprise administering a composition comprising, consisting of, or consisting essentially of a flow modulator (e.g., VEGFR3 agonist and/or FGF2) to a meningeal space of the subject, in which fluid flow in the central nervous system of the subject is increased, and administering a neurological therapeutic agent (e.g., amyloid beta antibody) to the subject. Thus, the method or use can increase the clearance of molecules from the CNS of the subject. The neurological therapeutic agent may be administered to the meningeal space, or to a different location in the subject. Increased clearance of molecules from the CNS of the subject can comprise an increased rate of movement of molecules from the CSF to deep cervical lymph nodes, and thus can be ascertained by monitoring the rate of movement of molecules and/or labels in the CNS to deep cervical lymph nodes. In some embodiments, the flow modulator (e.g., VEGFR3 agonist and/or FGF2) and/or the neurological therapeutic agent is administered selectively to the meningeal space. In some embodiments, a composition comprising, consisting of, or consisting essentially of the flow modulator (e.g., VEGFR3 agonist and/or FGF2) and/or the neurological therapeutic agent is administered to the CNS, but not outside the CNS. In some embodiments, the flow modulator (e.g., VEGFR3 agonist and/or FGF2) is administered to the CNS, but not blood. By way of example, the VEGFR3 agonist can be selected from the group consisting of one or more of the following: VEGF-c, VEGF-d, or an analog, variant, or functional fragment thereof.

Without being limited by theory, it is contemplated that, according to several embodiments herein, increasing flow by increasing the diameter of, increasing drainage by, and/or increasing the quantity of meningeal lymphatic vessels as described herein can increase clearance of molecules from the CNS of the subject, and thus reduces the concentration and/or accumulation of the molecules in the CNS and brain in accordance with some embodiments herein. Accordingly, in some embodiments, increasing clearance of molecules in the CNS reduces concentration and/or accumulation of the molecules in the CNS and brain. For example, if amyloid beta plaques are present in the CNS of the subject, increasing clearance can reduce amyloid beta plaques, or decrease the rate of their accumulation. Without being limited by theory, it is contemplated that by clearing soluble amyloid beta from the CNS, a gradient will favor solubilization of amyloid beta plaques, so that fluids in the CNS continue to flow and the CNS continues to be cleared, amyloid beta plaques can diminish, or the rate of increase can be reduced. Thus, decreases of amyloid beta plaques can represent a decrease in an etiology of a disease caused by amyloid beta plaques, and, more generally can indicate an increase in fluid flow in the CNS, for example via drainage by meningeal lymphatic vessels. In some embodiments, a quantity of accumulated amyloid beta plaques in the central nervous system, or the rate of accumulation thereof, is reduced by at least 2%, for example at least 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 100% including ranges between any two of the listed values, following administration of the flow modulator (e.g., VEGFR3 agonist and/or FGF2) and the neurological therapeutic agent (e.g., amyloid beta antibody). In some embodiments, amyloid beta plaques are cleared from meningeal portions of the central nervous system of the subject. In some embodiments, increased fluid flow in the central nervous system of the subject comprises an increased rate of perfusion of fluid throughout an area of the subject's brain. In some embodiments, increased fluid flow in the central nervous system of the subject comprises an increased rate of perfusion out of the subject's central nervous system.

As discussed herein, methods, uses, and compositions for increasing clearance of molecules from the CNS can be useful in treating, preventing, or ameliorating symptoms of neurological diseases, for example diseases associated with accumulation of macromolecules, cells, or debris in the CNS. Accordingly, in some embodiments, the method or use further includes determining the subject to have such a neurological disease, or a risk factor for such a neurological disease. Example neurological diseases include AD (such as familial AD and/or sporadic AD), PD, cerebral edema, ALS, PANDAS, meningitis, hemorrhagic stroke, ASD, brain tumor (such as glioblastoma), epilepsy, Down's syndrome, hereditary cerebral hemorrhage with amyloidosis-Dutch type (HCHWA-D), Familial Danish/British dementia, dementia with Lewy bodies (DLB), Lewy body (LB) variant of AD, multiple system atrophy (MSA), familial encephalopathy with neuroserpin inclusion bodies (FENIB), frontotemporal dementia (FTD), Huntington's disease (HD), Kennedy disease/spinobulbar muscular atrophy (SBMA), dentatorubropallidoluysian atrophy (DRPLA); spinocerebellar ataxia (SCA) type I, SCA2, SCA3 (Machado-Joseph disease), SCA6, SCA7, SCA17, Creutzfeldt-Jakob disease (CJD) (such as familial CJD), Kuru, Gerstmann-Straussler-Scheinker syndrome (GSS), fatal familial insomnia (FFI), corticobasal degeneration (CBD), progressive supranuclear palsy (PSP), cerebral amyloid angiopathy (CAA), multiple sclerosis (MS), AIDS-related dementia complex, brain injury, traumatic brain injury, brain hemorrhage, subarachnoid brain hemorrhage, stroke, or a combination of two or more of any of the listed items. In some embodiments, the neurological disease comprises, consists essentially of, or consists of a proteinopathy, for example a tauopathy or an amyloidosis such as AD (e.g., familial AD and/or sporadic AD).

In some embodiments, for any of the methods, compositions, or uses for increasing flow, increasing clearance, increasing drainage, increasing meningeal lymphatic diameter, and/or reducing amyloid beta plaques noted herein a FGF2 or a VEGFR3 agonist as described herein, and a neurological therapeutic agent can be administered. In some embodiments, the VEGFR3 agonist is selected from the group consisting of one or more of the following: VEGF-c, VEGF-d, or an analog, variant or functional fragment of either of these, and the neurological therapeutic agent comprises, consists essentially of, or consists of an amyloid beta antibody. In some embodiments, the VEGFR3 agonist and/or FGF2 and/or the neurological therapeutic agent (e.g., amyloid beta antibody) is administered selectively to the meningeal space of the subject. In some embodiments, the VEGFR3 agonist and/or FGF2 and/or the neurological therapeutic agent (e.g., amyloid beta antibody) is administered to the subject by a route selected from the group consisting of at least one of the following: nasal administration, transcranial administration, contact cerebral spinal fluid (CSF) of the subject, pumping into CSF of the subject, implantation into the skull or brain, contacting a thinned skull or skull portion of the subject with the VEGFR3 agonist and/or FGF2 and the neurological therapeutic agent (e.g., amyloid beta antibody), or expression in the subject of a nucleic acid encoding the VEGFR3 agonist and/or FGF2, or a combination of any of the listed routes. In some embodiments, the VEGFR3 agonist and/or FGF2 is administered to the subject after determining the subject to have the risk factor for the neurological disease. In some embodiments, the VEGFR3 agonist and/or FGF2 is administered to the subject after determining the subject to have the neurological disease. The VEGFR3 agonist and/or FGF2 and the neurological therapeutic agent (e.g., amyloid beta antibody) can be administered in an effective amount to treat, inhibit, ameliorate symptoms of, delay the onset of, reduce the likelihood of, prevent the neurological disease.

In certain embodiments, the increase of clearance of molecules can be evaluated by the increase of certain molecules, e.g., a protein, in the deep cervical lymph nodes, or the reduction of the molecule in the brain. Methods to detect and quantify a molecule, e.g., protein, are known in the art. For example, an imaging probe that is specific for the molecule, such as an antibody specific for such molecule conjugated with magnetic or fluorescent nanostructure, can be administered to the target tissue, e.g., brain or deep cervical lymph nodes. The image probe can be detected in vivo using methods known in the art, such as molecular magnetic resonance imaging as described in Viola et al., Towards non-invasive diagnostic imaging of early-stage Alzheimer's disease, Nature Nanotechnology, 10, 91-98 (2015). In some embodiments, the protein in deep cervical lymph nodes may be quantified from a biopsy sample.

Methods, Compositions, and Uses of Increasing Clearance of Cells from the CNS

In some aspect, the present invention is based upon, at least in part, the discovery that the number of immune cells increase in the central nervous system of a subject that has an enhance risk of developing a neurological disease, e.g., Alzheimer's disease. Accordingly, in some embodiments, the present invention provides a method of reducing the number of immune cells in the central nervous system of a subject.

Some embodiments of the disclosure include a method, use, or composition for use in increasing clearance of cells (such as immune cells) from the central nervous system of a subject. The method or use can comprise administering a composition comprising, consisting of, or consisting essentially of a flow modulator (e.g., VEGFR3 agonist and/or FGF2) to a meningeal space of the subject, in which fluid flow in the central nervous system of the subject is increased, and administering a neurological therapeutic agent to the subject.

Thus, the method or use can increase the clearance of cells from the CNS of the subject. The neurological therapeutic agent may be administered to the meningeal space, or to a different location in the subject. Increased clearance of cells from the CNS of the subject can comprise an increased rate of movement of cells from the CSF to deep cervical lymph nodes, and thus can be ascertained by monitoring the rate of movement of cells and/or labels in the CNS to deep cervical lymph nodes. In some embodiments, the flow modulator (e.g., VEGFR3 agonist and/or FGF2) and/or the neurological therapeutic agent is administered selectively to the meningeal space. In some embodiments, a composition comprising, consisting of, or consisting essentially of the flow modulator (e.g., VEGFR3 agonist and/or FGF2) and/or the neurological therapeutic agent is administered to the CNS, but not outside the CNS. In some embodiments, the flow modulator (e.g., VEGFR3 agonist and/or FGF2) is administered to the CNS, but not blood. By way of example, the VEGFR3 agonist can be selected from the group consisting of one or more of the following: VEGF-c, VEGF-d, or an analog, variant, or functional fragment thereof.

According to several embodiments herein, increasing flow by increasing the diameter of, increasing drainage by, and/or increasing the quantity of meningeal lymphatic vessels as described herein can increase clearance of cells, e.g., from the CNS of the subject, and thus reduces the concentration and/or accumulation of the cells, e.g., immune cells, in the CNS and brain in accordance with some embodiments herein. Accordingly, in some embodiments, increasing clearance of cells, e.g., immune cells, in the CNS reduces concentration and/or accumulation of the cells, e.g., immune cells, in the CNS and brain. Accordingly, the flow modulators (e.g., VEGF-c) of the invention synergize with the neurological therapeutic agents of the invention to reduce the number of cells that contribute to the pathogenesis of a neurodegenerative disease, e.g., Alzheimer's disease, resulting in a greater anti-inflammatory effect than either flow modulator or therapeutic agent alone.

Immune cells may contribute to the pathogenesis of a neurodegenerative disease through chronic inflammation. For example, immune cells such as T cells and B cells may contribute to chronic inflammation through secretion of proinflammatory cytokines. Without being limited by theory, it is contemplated that by clearing immune cells from the CNS, the neuroinflammation associated with neurodegenerative diseases, such as Alzheimer's disease, may be reduced and ameliorate the symptoms of the disease. Thus, decreases of immune cells can represent a decrease in an etiology of a disease caused by chronic inflammation, and, more generally can indicate an increase in fluid flow in the CNS, for example via drainage by meningeal lymphatic vessels. In some embodiments, a quantity of immune cells in the central nervous system, or the rate of accumulation thereof, is reduced by at least 2%, for example at least 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 100% including ranges between any two of the listed values, following administration of the flow modulator (e.g., VEGFR3 agonist and/or FGF2) and the neurological therapeutic agent. In some embodiments, amyloid beta plaques are cleared from meningeal portions of the central nervous system of the subject. In some embodiments, increased fluid flow in the central nervous system of the subject comprises an increased rate of perfusion of fluid throughout an area of the subject's brain. In some embodiments, increased fluid flow in the central nervous system of the subject comprises an increased rate of perfusion out of the subject's central nervous system.

As discussed herein, methods, uses, and compositions for increasing clearance of cells, e.g., immune cells, from the CNS can be useful in treating, preventing, or ameliorating symptoms of neurological diseases, for example diseases associated with accumulation of macromolecules, cells, or debris in the CNS. Accordingly, in some embodiments, the method or use further includes determining the subject to have such a neurological disease, or a risk factor for such a neurological disease. Example neurological diseases include AD (such as familial AD and/or sporadic AD), PD, cerebral edema, ALS, PANDAS, meningitis, hemorrhagic stroke, ASD, brain tumor (such as glioblastoma), epilepsy, Down's syndrome, hereditary cerebral hemorrhage with amyloidosis-Dutch type (HCHWA-D), Familial Danish/British dementia, dementia with Lewy bodies (DLB), Lewy body (LB) variant of AD, multiple system atrophy (MSA), familial encephalopathy with neuroserpin inclusion bodies (FENIB), frontotemporal dementia (FTD), Huntington's disease (HD), Kennedy disease/spinobulbar muscular atrophy (SBMA), dentatorubropallidoluysian atrophy (DRPLA); spinocerebellar ataxia (SCA) type I, SCA2, SCA3 (Machado-Joseph disease), SCA6, SCA7, SCA17, Creutzfeldt-Jakob disease (CJD) (such as familial CJD), Kuru, Gerstmann-Straussler-Scheinker syndrome (GSS), fatal familial insomnia (FFI), corticobasal degeneration (CBD), progressive supranuclear palsy (PSP), cerebral amyloid angiopathy (CAA), multiple sclerosis (MS), AIDS-related dementia complex, brain injury, traumatic brain injury, brain hemorrhage, subarachnoid brain hemorrhage, stroke, or a combination of two or more of any of the listed items. In some embodiments, the neurological disease comprises, consists essentially of, or consists of a proteinopathy, for example a tauopathy or an amyloidosis such as AD (e.g., familial AD and/or sporadic AD).

In some embodiments, for any of the methods, compositions, or uses for increasing flow, increasing clearance, increasing drainage, increasing meningeal lymphatic diameter, and/or reducing immune cells noted herein a FGF2 or a VEGFR3 agonist as described herein, and a neurological therapeutic agent can be administered. In some embodiments, the VEGFR3 agonist is selected from the group consisting of one or more of the following: VEGF-c, VEGF-d, or an analog, variant or functional fragment of either of these, and the neurological therapeutic agent comprises, consists essentially of, or consists of an amyloid beta antibody. In some embodiments, the VEGFR3 agonist and/or FGF2 and/or the neurological therapeutic agent is administered selectively to the meningeal space of the subject. In some embodiments, the VEGFR3 agonist and/or FGF2 and/or the neurological therapeutic agent is administered to the subject by a route selected from the group consisting of at least one of the following: nasal administration, transcranial administration, contact cerebral spinal fluid (CSF) of the subject, pumping into CSF of the subject, implantation into the skull or brain, contacting a thinned skull or skull portion of the subject with the VEGFR3 agonist and/or FGF2 and the neurological therapeutic agent, or expression in the subject of a nucleic acid encoding the VEGFR3 agonist and/or FGF2, or a combination of any of the listed routes. In some embodiments, the VEGFR3 agonist and/or FGF2 is administered to the subject after determining the subject to have the risk factor for the neurological disease. In some embodiments, the VEGFR3 agonist and/or FGF2 is administered to the subject after determining the subject to have the neurological disease. The VEGFR3 agonist and/or FGF2 and the neurological therapeutic agent can be administered in an effective amount to treat, inhibit, ameliorate symptoms of, delay the onset of, reduce the likelihood of, prevent the neurological disease.

Methods, Compositions, and Uses of Reducing Microglial Inflammatory Response in the CNS

Some embodiments include methods, compositions for use, and uses for reducing a microglial inflammatory response in the central nervous system of a subject. In one embodiment, the microglial inflammatory response is a response towards a protein aggregate, such as such as amyloid beta, fibrin, tau, or alpha-synuclein aggregates. In one embodiment, the protein aggregate comprises amyloid beta plaques. Accordingly, the present invention provides compositions and methods for reducing a microglial inflammatory response in the central nervous system. In some embodiment, the microglial inflammatory response is located in the brain of the subject.

The methods or uses can include administering a flow modulator (e.g., VEGFR3 agonist and/or FGF2) to a meningeal space of the subject, so that fluid flow (e.g., flow of ISF, CSF, or both) in the central nervous system of the subject is increased, and further administering and a neurological therapeutic agent to the subject (the neurological therapeutic agent can be administered to a meningeal space or to a different location), thereby reducing the microglial inflammatory response in the central nervous system of the subject. By way of example, the VEGFR3 agonist can comprise (or consist essentially of, or consist of) VEGF-c, and the neurological therapeutic agent can comprise (or consist essentially of, or consist of) an amyloid beta antibody. The reduction of microglial inflammatory response can be measured by the reduced number of microglia surrounding a protein aggregate, e.g., amyloid beta plaque, or by the reduced percentage of activated microglia to the total number of microglia. In some embodiments, the quantity of microglia surrounding a protein aggregate, e.g., an amyloid beta plaque, or the percentage of activated microglia to the total microglia, or both is reduced by at least 2%, for example, at least 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 100% including ranges between any two of the listed values. In some embodiments, the flow modulator (e.g., VEGFR3 agonist and/or FGF2) and/or the neurological therapeutic agent is administered selectively to the meningeal space. In some embodiments, the flow modulator (e.g., VEGFR3 agonist and/or FGF2) and/or the neurological therapeutic agent is administered to the CNS, but not outside the CNS. In some embodiments, the flow modulator (e.g., VEGFR3 agonist and/or FGF2) and/or the neurological therapeutic agent is administered to the CNS, but not blood. In some embodiments, the VEGFR3 agonist is selected from the group consisting of at least one of the following: VEGF-c, VEGF-d, or an analog, variant, or functional fragment thereof. In some embodiments, the neurological therapeutic agent comprises, consists essentially of, or consists of an amyloid beta antibody.

Methods, Compositions, and Uses of Reducing Neurite Dystrophy in the CNS

Some embodiments include methods, compositions for use, and uses for reducing a neurite dystrophy in the central nervous system of a subject. In one embodiment, the neurite dystrophy is caused by or associated with a protein aggregate, such as such as amyloid beta, fibrin, tau, or alpha-synuclein aggregates. In one embodiment, the protein aggregate comprises amyloid beta plaques. Accordingly, the present invention provides compositions and methods for reducing a neurite dystrophy in the central nervous system. In some embodiments, the neurite dystrophy is located in the brain of the subject.

The methods or uses can include administering a flow modulator (e.g., VEGFR3 agonist and/or FGF2) to a meningeal space of the subject, so that fluid flow (e.g., flow of ISF, CSF, or both) in the central nervous system of the subject is increased, and further administering and a neurological therapeutic agent to the subject (the neurological therapeutic agent can be administered to a meningeal space or to a different location), thereby reducing the neurite dystrophy in the central nervous system of the subject. By way of example, the VEGFR3 agonist can comprise (or consist essentially of, or consist of) VEGF-c, and the neurological therapeutic agent can comprise (or consist essentially of, or consist of) an amyloid beta antibody. The reduction of neurite dystrophy can be measured by the reduced level of lysosomal-associated membrane protein 1 positive cells. In some embodiments, the neurite dystrophy is reduced by at least 2%, for example, at least 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 100% including ranges between any two of the listed values. In some embodiments, the flow modulator (e.g., VEGFR3 agonist and/or FGF2) and/or the neurological therapeutic agent is administered selectively to the meningeal space. In some embodiments, the flow modulator (e.g., VEGFR3 agonist and/or FGF2) and/or the neurological therapeutic agent is administered to the CNS, but not outside the CNS. In some embodiments, the flow modulator (e.g., VEGFR3 agonist and/or FGF2) and/or the neurological therapeutic agent is administered to the CNS, but not blood. In some embodiments, the VEGFR3 agonist is selected from the group consisting of at least one of the following: VEGF-c, VEGF-d, or an analog, variant, or functional fragment thereof. In some embodiments, the neurological therapeutic agent comprises, consists essentially of, or consists of an amyloid beta antibody.

Methods, Compositions, and Uses of Reducing Vascular Fibrinogen Deposition in the CNS

Some embodiments include methods, compositions for use, and uses for reducing a vascular fibrinogen deposition in the central nervous system of a subject. In one embodiment, the vascular fibrinogen deposition is a response towards a protein aggregate, such as such as amyloid beta, fibrin, tau, or alpha-synuclein aggregates. In one embodiment, the protein aggregate comprises amyloid beta plaques. Accordingly, the present invention provides compositions and methods for reducing vascular fibrinogen deposition in the central nervous system. In some embodiment, the vascular fibrinogen deposition is located in the brain of the subject.

The methods or uses can include administering a flow modulator (e.g., VEGFR3 agonist and/or FGF2) to a meningeal space of the subject, so that fluid flow (e.g., flow of ISF, CSF, or both) in the central nervous system of the subject is increased, and further administering and a neurological therapeutic agent to the subject (the neurological therapeutic agent can be administered to a meningeal space or to a different location), thereby reducing the vascular fibrinogen deposition in the central nervous system of the subject. By way of example, the VEGFR3 agonist can comprise (or consist essentially of, or consist of) VEGF-c, and the neurological therapeutic agent can comprise (or consist essentially of, or consist of) an amyloid beta antibody. The reduction of vascular fibrinogen deposition can be measured by the reduced quantity of fibrinogen fibers surrounding a protein aggregate, e.g., amyloid beta plaque. In some embodiments, the quantity of vascular fibrinogen deposition surrounding a protein aggregate, e.g., an amyloid beta plaque, is reduced by at least 2%, for example, at least 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 100% including ranges between any two of the listed values. In some embodiments, the flow modulator (e.g., VEGFR3 agonist and/or FGF2) and/or the neurological therapeutic agent is administered selectively to the meningeal space. In some embodiments, the flow modulator (e.g., VEGFR3 agonist and/or FGF2) and/or the neurological therapeutic agent is administered to the CNS, but not outside the CNS. In some embodiments, the flow modulator (e.g., VEGFR3 agonist and/or FGF2) and/or the neurological therapeutic agent is administered to the CNS, but not blood. In some embodiments, the VEGFR3 agonist is selected from the group consisting of at least one of the following: VEGF-c, VEGF-d, or an analog, variant, or functional fragment thereof. In some embodiments, the neurological therapeutic agent comprises, consists essentially of, or consists of an amyloid beta antibody.

### Additional Embodiments

All technical and scientific terms used herein have the meaning as would be understood by one of ordinary skill in the art to which this subject matter belongs, in view of this disclosure.

It is appreciated that certain features, which are, for clarity, described in the context of separate embodiments, may also be provided in combination in a single embodiment. Conversely, various features, which are, for brevity, described in the context of a single embodiment, may also be provided separately or in any suitable sub-combination. All combinations of the embodiments pertaining to the subject matter herein are specifically contemplated and are disclosed herein just as if each and every combination was individually and explicitly disclosed. In addition, all sub-combinations of the various embodiments and elements thereof are also specifically contemplated and are disclosed herein just as if each and every such sub-combination was individually and explicitly disclosed herein.

Some aspects provide methods of treating a neurological disease (such as AD) in a subject comprising administering to the subject a therapeutically effective amount of a neurological therapeutic agent and a flow modulator that modulates one or more of a) drainage of the meningeal lymphatic vessel(s); b) diameter of the meningeal lymphatic vessel(s); c) lymphangiogenesis of the meningeal lymphatic vessel(s); d) contractility of the meningeal lymphatic vessel(s); and/or e) permeability of the meningeal lymphatic vessel(s). The present disclosure also provides methods of treating AD in a subject by administering to the subject a neurological therapeutic agent and a flow modulator that increases drainage of the meningeal lymphatic vessel(s), increases the diameter of the meningeal lymphatic vessel(s), causes lymphangiogenesis of the meningeal lymphatic vessel(s), modulates contractility of the meningeal lymphatic vessel(s) to increase drainage, and/or modulates the permeability of the meningeal lymphatic vessel(s) to increase drainage. The present disclosure also provides methods of treating a neurological disease such as AD described herein in a subject by administering to the subject a neurological therapeutic agent and a flow modulator that increases drainage of the meningeal lymphatic vessel(s), increases the diameter of the meningeal lymphatic vessel(s), causes lymphangiogenesis of the meningeal lymphatic vessel(s), modulates contractility of the meningeal lymphatic vessel(s) to increase drainage, and/or modulates the permeability of the meningeal lymphatic vessel(s) to increase drainage.

Below are non-limiting examples of some embodiments herein:

### EXAMPLES

### Example 1: Adult 5×FAD Meninges

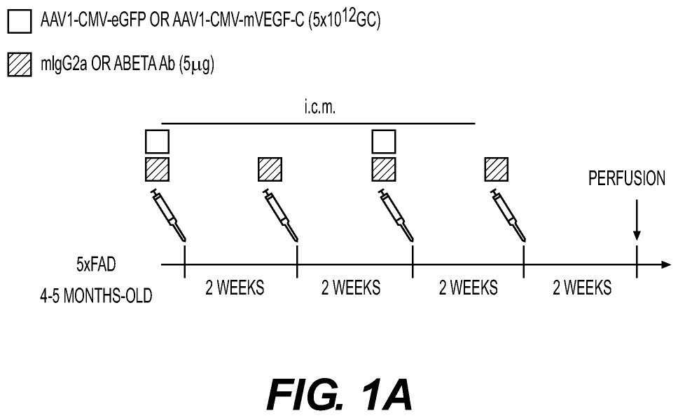

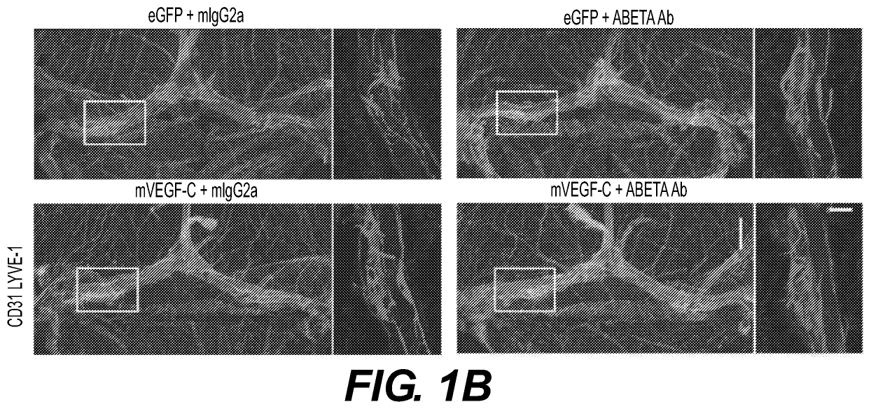

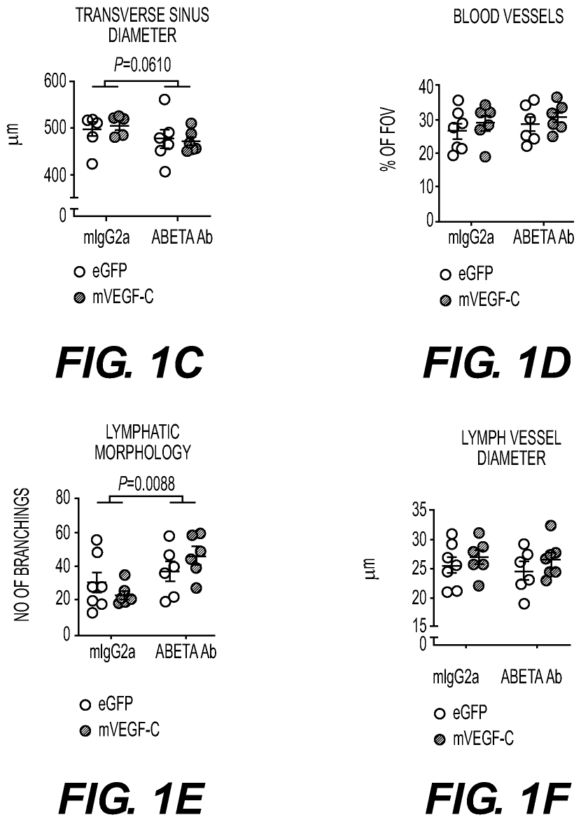

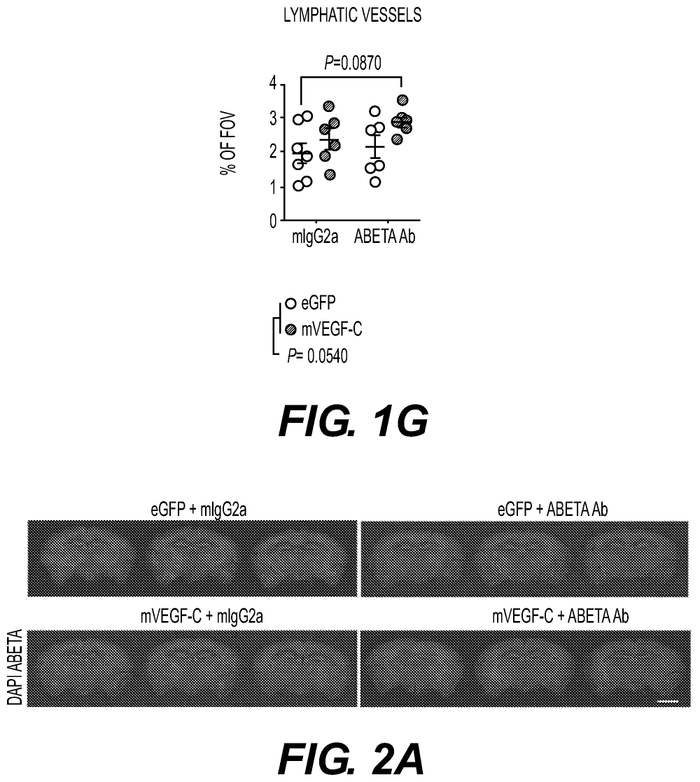

Adult 5×FAD mice were treated with a combination of VEGF-c and an antibody that binds amyloid beta. The age of the of 5×FAD mice and treatment regimen are summarized in FIG. 1A). After administration of the VEGF-c and the antibody, brain and meningeal morphology was observed. Representative images of the meningeal whole-mounts of 5×FAD mice treated with different combinations of mIgG2a or monoclonal anti-amyloid beta antibody (“ABETA Ab”) with AAV1-CMV-eGFP or AAV1-CMV-mVEGF-C are shown in FIG. 1B. Meninges were stained for LYVE-1 (red) and CD31 (green); scale bar, 1 mm; inset, 300 μm (FIG. 1B) Measurements of transverse sinus diameter, coverage by LYVE-1negCD31+ blood vessels, total number of lymphatic branches, transverse sinus lymphatic vessel diameter and coverage by LYVE-1+ lymphatic vessels (See FIGS. 1C-1G). Results in FIGS. 1C-1G are presented as mean±s.e.m.; n=7 in eGFP+mIgG2a, n=6 in eGFP+ ABETA Ab, in mVEGF-C+mIgG2a and in mVEGF-C+ ABETA Ab; Two-way ANOVA with Sidak's multiple comparison test. In FIGS. 1D and 1G, the units for the Y-axis are percentage of field of view (“% FOV”). It was shown that the flow modulator VEGF-c increased lymphatic diameter, and synergized with the amyloid beta antibody to enhance lymphatic branching. These experiments show that a flow modulator and neurological therapeutic agent in accordance with some embodiments herein can synergize to enhance lymphatic branching in the CNS of a model of AD comprising amyloid beta plaques.

### Example 2: Adult 5×FAD Brain

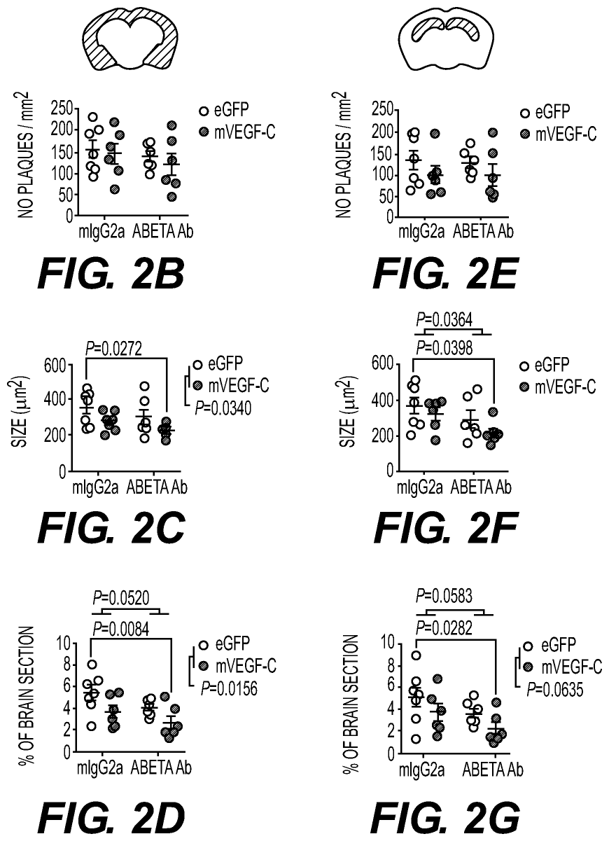

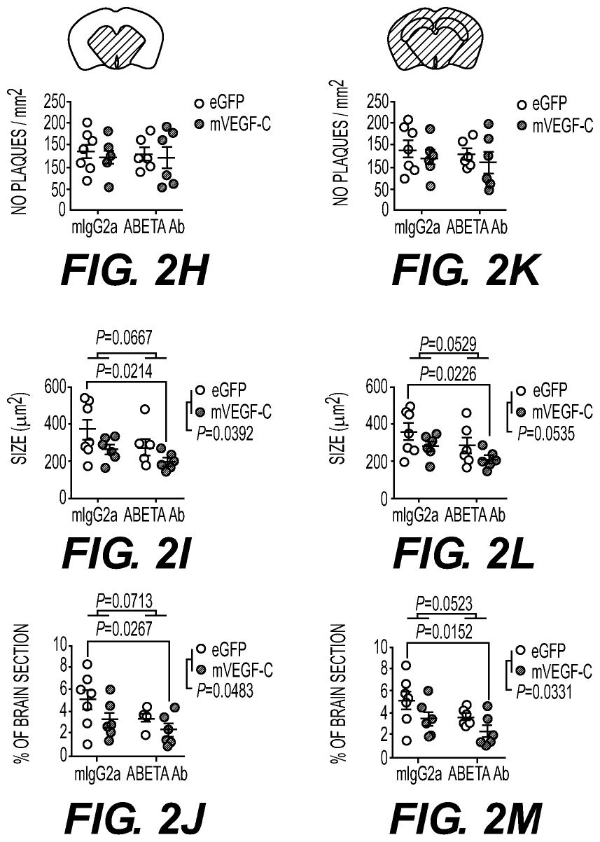

Adult 5×FAD mice were treated with a combination of VEGF-c and an antibody that binds amyloid beta. Representative images of the brain sections of 5×FAD mice treated with different combinations of mIgG2a or ABETA Ab with AAV1-CMV-eGFP or AAV1-CMV-mVEGF-C. Representative images of the brain sections of these mice are shown in FIG. 2A. Brain sections were stained for Aβ (red) and with DAPI (blue); scale bar, 2 mm. b-m, Plaque density (number of plaques per mm²), average size (μm²) and coverage (% of brain section) in particular brain regions (cortex and amygdala; hippocampus; thalamus and hypothalamus) or in the whole brain section. Results in FIGS. 2B-2M are presented as mean±s.e.m.; n=7 in eGFP+mIgG2a, n=6 in eGFP+ ABETA Ab, in mVEGF-C+mIgG2a and in mVEGF-C+ ABETA Ab; Two-way ANOVA with Sidak's multiple comparison test. These experiments show that a flow modulator and neurological therapeutic agent in a model of AD comprising amyloid beta plaques in accordance with some embodiments herein can synergize to reduce amyloid beta plaque size, density, and coverage (as percent of brain region).

### Example 3: Old APPswe Behavior and Brain

Age of APPswe mice, treatment regimen and behavioral tests: open field (OF), novel location recognition (NLR) and contextual fear conditioning (CFC) are shown in FIG. 3A. Total distance, velocity and time in center of the arena (% of total time) in the OF test are shown in FIG. 3B. Time investigating one of the object location (% of total time investigating objects) in the training trial and time investigating the novel object location (% of total time investigating) in the NLR test are shown in FIG. 3C. Time freezing (% of total time) in the context trial and in cued trial of the CFC are shown in FIG. 3D. Representative images of the brain sections of APPswe mice treated with anti-Abeta antibody and with AAV1-CMV-eGFP or AAV1-CMV-mVEGF-C are shown in FIG. 3E. Brain sections were stained for Aβ (red) and with DAPI (blue); scale bar, 1 mm. FIGS. 3F-3G show plaque density (number of plaques per mm²), average size (μm²) and coverage (% of brain section) in the cortex and amygdala or in the hippocampus. Results in FIGS. 3B-3D, 3F, and 3G are presented as mean±s.e.m.; n=11 per group; Two-tailed unpaired Student's T test.

### Example 4: J20 Amyloid Plaques Brain

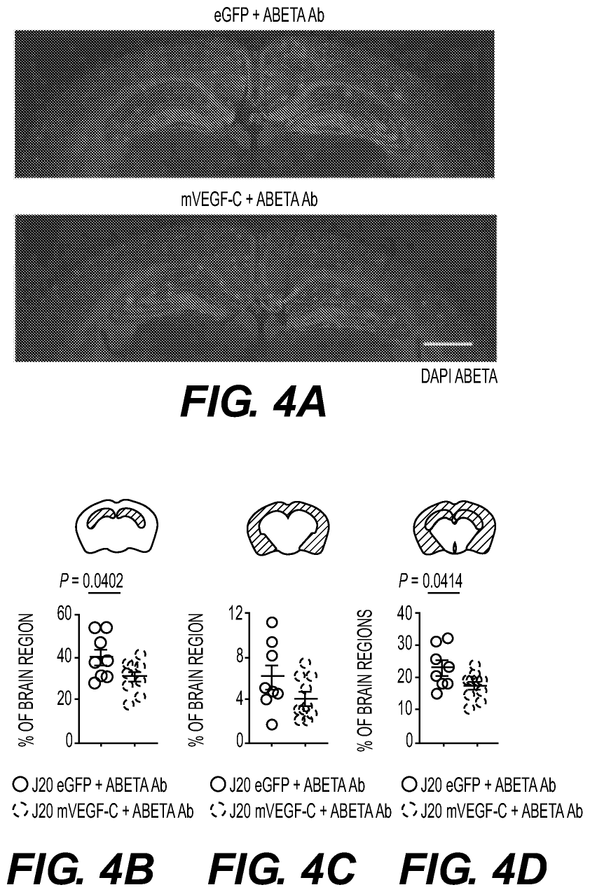

J20 mice were treated with a combination of VEGF-c and an antibody that binds amyloid beta. Representative images of the brain sections of J20 mice treated with ABETA Ab and with the antibody and either AAV1-CMV-eGFP or AAV1-CMV-mVEGF-C are shown in FIG. 4A. Brain sections were stained for Aβ (red) and with DAPI (blue). FIGS. 4B-4D show amyloid beta plaque coverage (% of brain section) in the hippocampus, cortex and amygdala, or in the hippocampus, cortex and amygdala combined. These experiments show that a flow modulator and neurological therapeutic agent in an additional model of AD (J20 mice) comprising amyloid beta plaques in accordance with some embodiments herein can synergize to reduce amyloid beta plaque coverage (as percent of brain region).

### Example 5: Treatment with VEGF-c and Aducanumab

A human subject is identified as suffering from Alzheimer's disease. A pharmaceutical composition comprising human VEGF-c and aducanumab is administered monthly to the subject via intravenous infusion. The intravenous administration is repeated monthly for eight months. Amelioration of behavior symptoms of Alzheimer's disease, including memory loss is observed. Reduction in quantity of density of amyloid beta plaques in the brain of the subject is observed by in vivo magnetic resonance imaging.

### Example 6: Alzheimer's Disease

The prevalence of AD and other dementias is expected to increase owing to better health care and higher life expectancy. Increased accumulation and aggregation of AD, the main constituent of senile plaques in the brain parenchyma, is one of the key pathological hallmarks of AD (Benilova, I., Karran, E. & De Strooper, B. The toxic Abeta oligomer and Alzheimer's disease: an emperor in need of clothes. Nat Neurosci 15, 349-357, (2012); Bateman, R. J. et al. Clinical and biomarker changes in dominantly inherited Alzheimer's disease. N Engl J Med 367, 795-804, (2012)). Pathological accumulation of Aβ in the brain results, in part, from the age-related progressive impairment of cleansing mechanisms (Mawuenyega, K. G. et al. Decreased clearance of CNS beta-amyloid in Alzheimer's disease. Science 330, 1774, (2010); Tarasoff-Conway, J. M. et al. Clearance systems in the brain—implications for Alzheimer diseaser. Nat Rev Neurol 12, 248, (2016)), including the meningeal lymphatic vasculature (Da Mesquita, S. et al. Functional aspects of meningeal lymphatics in ageing and Alzheimer's disease. Nature 560, 185-191, (2018); Da Mesquita, S., Fu, Z. & Kipnis, J. The Meningeal Lymphatic System: A New Player in Neurophysiology. Neuron 100, 375-388, (2018)). Passive immunotherapy, using monoclonal antibodies against Aβ, is among the most promising of the therapeutic strategies aimed at enhancing the clearance of toxic Aβ species from the brain (Bacskai, B. J. et al. Imaging of amyloid-beta deposits in brains of living mice permits direct observation of clearance of plaques with immunotherapy. Nat Med 7, 369-372, (2001); Bard, F. et al. Peripherally administered antibodies against amyloid beta-peptide enter the central nervous system and reduce pathology in a mouse model of Alzheimer disease. Nat Med 6, 916-919, (2000); Sevigny, J. et al. Addendum: The antibody aducanumab reduces Abeta plaques in Alzheimer's disease. Nature 546, 564, (2017)). Two clinical trials of the anti-Aβ monoclonal antibody Aducanumab (Sevigny et al., supra), EMERGE and ENGAGE, have recently yielded somewhat contradictory results, since cognitive decline was significantly reduced in patients receiving the highest dose (10 mg/kg) in the EMERGE cohort but not in the ENGAGE cohort (Howard, R. & Liu, K. Y. Questions EMERGE as Biogen claims aducanumab turnaround. Nat Rev Neurol, (2019)). This controversy, along with the meager clinical improvement observed in patients with mild cognitive impairment and AD who were enlisted in trials involving other monoclonal antibodies (Salloway, S. et al. Two phase 3 trials of bapineuzumab in mild-to-moderate Alzheimer's disease. N Engl J Med 370, 322-333, (2014); Logovinsky, V. et al. Safety and tolerability of BAN2401—a clinical study in Alzheimer's disease with a protofibril selective Abeta antibody. Alzheimers Res Ther 8, 14, (2016)), highlights the need for a better understanding of possible factors that might influence the efficacy of anti-Aβ immunotherapy in AD.

It has been previously shown that induction of meningeal lymphatic dysfunction exacerbates brain and meningeal Aβ pathology in different transgenic mouse models of familial AD (Da Mesquita, S. et al. Functional aspects of meningeal lymphatics in ageing and Alzheimer's disease. Nature 560, 185-191, (2018)). Accordingly, it was hypothesized that altered meningeal lymphatic function would affect brain fluid drainage and recirculation, thereby changing the availability of monoclonal antibodies administered to target and clear brain Aβ deposits. This hypothesis led to further investigate changes in the meningeal lymphatic vasculature at different ages in transgenic mouse models of AD, as well as the potential therapeutic implications of modulating this brain-draining meningeal lymphatic system.

### Example 7: Meningeal Lymphatics Become Impaired in 5×FAD Mice

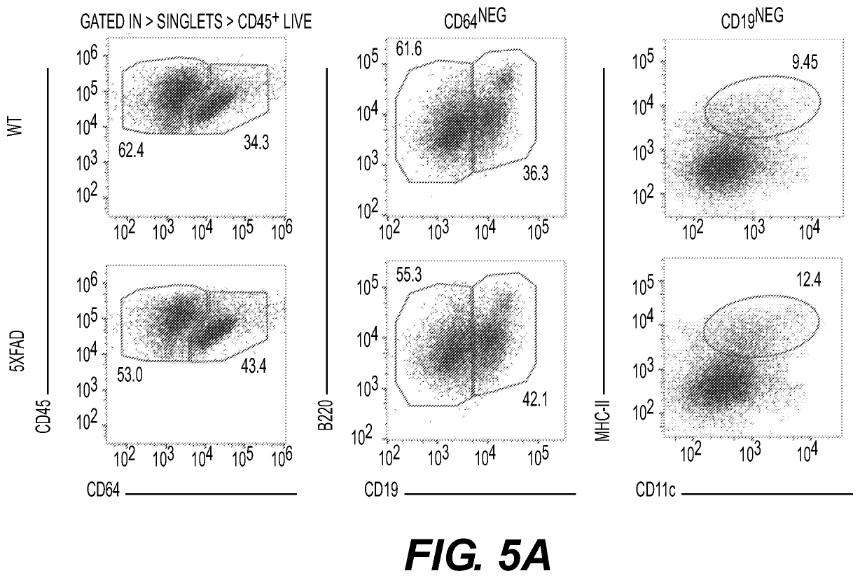

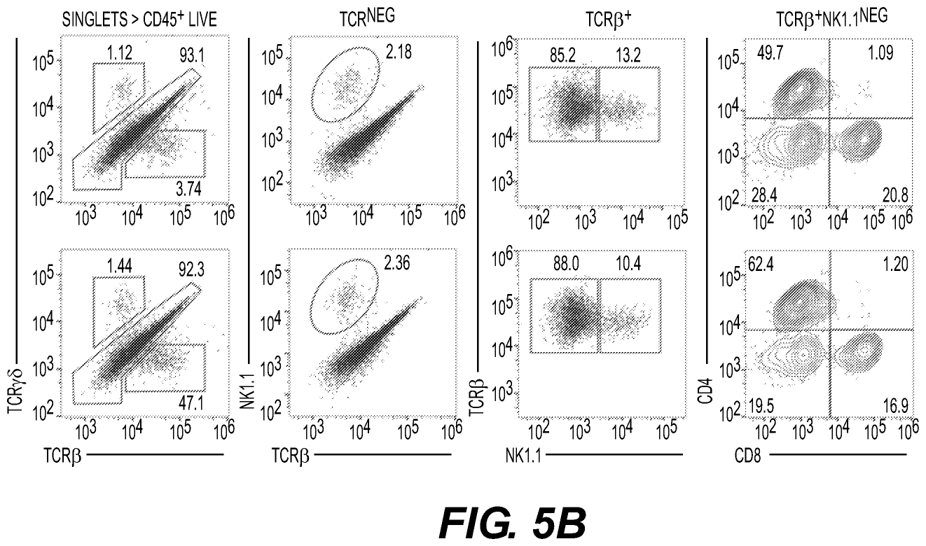

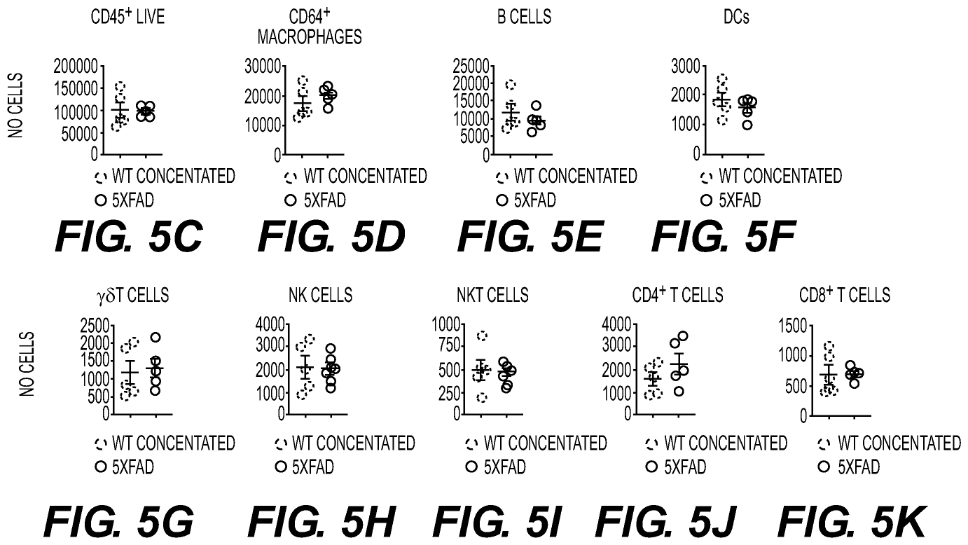

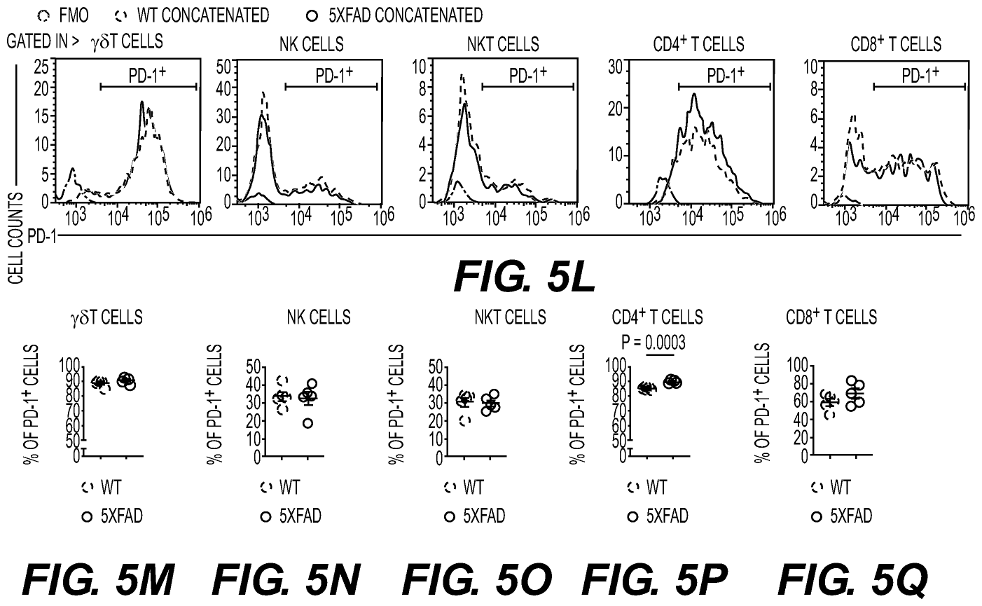

It was previously shown that young-adult (˜3 month-old) 5×FAD mice and age-matched wild-type (WT) littermates present no meningeal lymphatic dysfunction as assessed morphologically and functionally (Da Mesquita, S. et al. Functional aspects of meningeal lymphatics in ageing and Alzheimer's disease. Nature 560, 185-191, (2018)). Likewise, no major changes in the meningeal immune response are detectable at this young age (FIGS. 5A-5Q).

It was next sought to investigate whether changes in meningeal lymphatic vasculature and immunity would emerge with aging in these AD transgenic mice. This study began by comparing the meningeal lymphatic morphology in 3-, 6- and 14-month-old 5×FAD mice (FIG. 6A). In the meninges of the 6-month-old 5×FAD mice it was observed, surprisingly, an early loss of lymphatic vessel coverage along the superior sagittal sinus (FIGS. 6B and 6C). This was followed, as it was observed in the 14-month-old mice, by a marked decrease in lymphatic coverage at the dural confluence of sinuses, the transverse (TS), sigmoid (SS) and petrosquamosal (PSS) sinuses (FIGS. 6D and 6E). The early and progressive deterioration of the lymphatic vasculature was accompanied by a significant increase in meningeal Aβ deposition at 6 months, which was even more marked at 14 months (FIG. 6F). Interestingly, extensive meningeal Aβ deposition along the blood and lymphatic vasculature (FIG. 6A) was more evident at anatomical locations previously shown to be ‘hot spots’ for CSF access to the lymphatic vasculature at the dorsal meninges ensheathing the brain (Louveau, A. et al. CNS lymphatic drainage and neuroinflammation are regulated by meningeal lymphatic vasculature. Nat Neurosci 21, 1380-1391, (2018).

### Example 8: Meningeal Lymphatic Drainage Affects Anti-Aβ Immunotherapy

In this study, incubation of brain sections from WT or 5×FAD mice confirmed that anti-Abeta antibody specifically recognizes human Aβ deposited in the brains of 5×FAD mice, but not of WT mice (FIG. 7A). Murine IgG2a isotype antibody (mIgG2a, clone 4-4-20e), used here as a control, did not recognize AD plaques in brain sections from 5×FAD mice (FIG. 7A). To determine the efficient route for targeting of brain Aβ plaques, anti-Abeta antibody was injected into 5-month-old 5×FAD mice, either via the CSF (5 μL at 1 μg/L) by intra-cisterna magna (i.c.m.) infusion or intravenously (i.v., 100 μL at 1 μg/L). Both at 1 h and at 24 h after anti-Abeta antibody injection, the recognition of Aβ plaques was more pronounced when the anti-Abeta antibody was administered into the CSF (FIG. 7B) than via the i.v. route (FIG. 7C). The results, in agreement with a recent report (Plog, B. A. et al. Transcranial optical imaging reveals a pathway for optimizing the delivery of immunotherapeutics to the brain. JCI Insight 3, (2018)), suggest that bypassing the tight blood-brain barrier in adult 5×FAD mice, via direct delivery into the CSF, allows a better access of anti-Aβ antibodies to parenchymal Aβ aggregates. To determine whether delivery of anti-Abeta antibody into the CSF can in fact promote clearance of brain Aβ plaques, anti-Abeta antibody (0.5 or g) or mIgG2a (5 μg) was injected directly into the cisterna magna of 3-month-old 5×FAD mice every 2 weeks for 2 months (FIG. 8A). This regimen resulted in a significant reduction, across different brain regions, of Aβ plaque size in the anti-Abeta antibody-injected groups, with a stronger effect obtained for the higher dose of 5 μg (FIGS. 8B-8K).

Based on recent experimental evidence for an impaired perivascular CSF influx (via the glymphatic pathway) in mouse models of meningeal lymphatic dysfunction (Da Mesquita, S. et al. Functional aspects of meningeal lymphatics in ageing and Alzheimer's disease. Nature 560, 185-191, (2018); Zou, W. et al. Blocking meningeal lymphatic drainage aggravates Parkinson's disease-like pathology in mice overexpressing mutated alpha-synuclein. Transl Neurodegener 8, 7, (2019); Wang, L. et al. Deep cervical lymph node ligation aggravates AD-like pathology of APP/PS1 mice. Brain Pathol 29, 176-192, (2019)), it was postulated that exacerbation of meningeal lymphatic dysfunction in 5×FAD mice would dampen the clearance of Aβ plaques owing to reduced access of anti-Abeta antibody to the brain parenchyma. To test this, induced meningeal lymphatic dysfunction was introduced in WT or 5×FAD mice using a previously described method of lymphatic vessel photoablation (Da Mesquita, S. et al. Functional aspects of meningeal lymphatics in ageing and Alzheimer's disease. Nature 560, 185-191, (2018); Louveau, A. et al. CNS lymphatic drainage and neuroinflammation are regulated by meningeal lymphatic vasculature. Nat Neurosci 21, 1380-1391, (2018), achieved by injecting Visudyne into the CSF and five consecutive transcranial photoconversion steps of the drug by a 689-nm-wavelength nonthermal red light. Analysis of meningeal lymphatic morphology in WT mice 1 week later revealed efficient ablation of the lymphatic vasculature lining the transverse sinus (in the dorsal meninges, FIGS. 6G and 6H), but no significant changes in the continuing lymphatic vessels present around the SS and PSS (in the basal meninges, FIG. 6I). The fluorescent microsphere drainage from the CSF into the dCLNs in WT mice was also measured using in-vivo stereomicroscopic imaging of the collecting lymphatic vessel afferent to the dCLN (FIG. 6J). This revealed a significant reduction in CSF drainage 1 week after photoablation of the lymphatic vasculature at the dorsal meninges (FIG. 6K). These results reinforced previous findings emphasizing the important contribution of initial lymphatics present at the dorsal brain meninges (Da Mesquita, S. et al. Functional aspects of meningeal lymphatics in ageing and Alzheimer's disease. Nature 560, 185-191, (2018); Louveau, A. et al. CNS lymphatic drainage and neuroinflammation are regulated by meningeal lymphatic vasculature. Nat Neurosci 21, 1380-1391, (2018); Louveau, A. et al. Structural and functional features of central nervous system lymphatic vessels. Nature 523, 337-341, (2015)), namely along the transverse sinus, for drainage of CSF components into the dCLNs, and call into question a more recent assertion specifying a key role for the lymphatics in the basal region of the meninges (Ahn, J. H. et al. Meningeal lymphatic vessels at the skull base drain cerebrospinal fluid. Nature 572, 62-66, (2019)).

To test the effect of decreased meningeal lymphatic drainage on anti-Abeta antibody-mediated AD plaque clearance, meningeal lymphatic vessel ablation was induced in 4-5 months-old 5×FAD mice, the mice were allowed to recover for 1 week, and then anti-Abeta antibody was administered into the CSF. By the end of the anti-Abeta antibody treatment regimen (described in FIG. 6L), mice with intact meningeal lymphatic vasculature (FIGS. 9A-9E) presented significantly less Aβ plaque load than mice with ablated meningeal lymphatics across different brain regions, especially in the cortex (FIGS. 6M-6Q).

In an attempt to explain the reduced efficacy of anti-Abeta antibody observed in the mice with impaired meningeal lymphatic drainage, 1 hour after introducing antibodies into the CSF, assays were performed to measure the amounts of anti-Abeta antibody in the brain that were colocalized with CD31⁺ vessels or with Aβ aggregates (FIGS. 10A-10E). Interestingly, although there were no differences in the amount of anti-Abeta antibody in the brain vasculature (FIGS. 10B and 10C), significantly less anti-Abeta antibody was found to be colocalized with brain parenchymal Aβ aggregates in the 5×FAD mice with ablated meningeal lymphatic vasculature (FIGS. 10D and 10E). These findings suggested that impairment of meningeal lymphatic drainage in 5×FAD mice leads to decreased perivascular influx of anti-Abeta antibody from the CSF into the brain, reduced access of anti-Abeta antibody to brain parenchymal Aβ plaques, and less clearance of Aβ plaques by anti-Abeta antibody.

Most monoclonal anti-Aβ antibodies tested in clinical trials have failed to significantly prevent cognitive decline in AD patients, possibly owing to serious side effects such as deleterious activation of blood vasculature and microglia (Ryu, J. K. et al. Fibrin-targeting immunotherapy protects against neuroinflammation and neurodegeneration. Nat Immunol 19, 1212-1223, (2018); Merlini, M. et al. Fibrinogen Induces Microglia-Mediated Spine Elimination and Cognitive Impairment in an Alzheimer's Disease Model. Neuron 101, 1099-1108 e1096, (2019); Hong, S. et al. Complement and microglia mediate early synapse loss in Alzheimer mouse models. Science 352, 712-716, (2016)) or microhemorrhages in the brain and meninges (Salloway, S. et al. Two phase 3 trials of bapineuzumab in mild-to-moderate Alzheimer's disease. N Engl J Med 370, 322-333, (2014); Sperling, R. et al. Amyloid-related imaging abnormalities in patients with Alzheimer's disease treated with bapineuzumab: a retrospective analysis. Lancet Neurol 11, 241-249, (2012); Pfeifer, M. et al. Cerebral hemorrhage after passive anti-Abeta immunotherapy. Science 298, 1379, (2002)). It was postulated that improving meningeal lymphatic drainage would improve the efficacy of the anti-Abeta antibody and potentially reduce immunotherapy-associated side effects. To explore this possibility, a combination therapy was tested. Passive anti-Abeta antibody immunotherapy was combined with adeno-associated virus 1 (AAV1)-mediated expression of murine vascular endothelial growth factor-C (mVEGF-C, FIG. 11A). Increased VEGF-C signaling through VEGFR3 expressed by meningeal LECs was previously shown by research groups to improve meningeal lymphatic function and CSF drainage (Da Mesquita, S. et al. Functional aspects of meningeal lymphatics in ageing and Alzheimer's disease. Nature 560, 185-191, (2018); Louveau, A. et al. Structural and functional features of central nervous system lymphatic vessels. Nature 523, 337-341, (2015); Antila, S. et al. Development and plasticity of meningeal lymphatic vessels. J Exp Med 214, 3645-3667, (2017)). Introduction of mVEGF-C-expressing virus together with the anti-Abeta antibody into the CSF of 5×FAD mice (following the regimen described in FIG. 11A) resulted in significant expansion of lymphatic vasculature around the transverse sinus at the dorsal meninges when compared to that seen in the groups treated with anti-Abeta antibody and the control AAV1 (expressing enhanced green fluorescent protein, eGFP) or with mIgG2a and the AAV1s (FIGS. 11B-11D and FIGS. 12A-12C). There were no significant differences between the groups in terms of length or complexity of lymphatic vasculature in the basal region of the meninges (FIGS. 11B, 11E, and 11F), or the blood vessel coverage in meningeal whole mounts (FIGS. 12A and 12B). In mice treated with mVEGF-C and anti-Abeta antibody, measurements of brain Aβ plaque load, neurite dystrophy (assessed by the levels of lysosomal-associated membrane protein 1), vascular fibrinogen deposition, and myeloid (IBA1⁺) cell response at the end point of the treatment regimen (2 weeks after the last injection of mIgG2a or anti-Abeta antibody) revealed a significant reduction of AD plaque coverage throughout the entire forebrain (FIGS. 11G-11K), less cortical vascular fibrinogen (FIGS. 11L-11N), fewer IBA1⁺ cells clustering around Aβ deposits, and decreased amounts of CD68 in IBA1⁺ cells (FIGS. 11O-11R). Groups that received anti-Abeta antibody presented significantly less dystrophic neurites, regardless of the treatment with mVEGF-C (FIGS. 11L and 11M). In sum, combination therapy with mVEGF-C and anti-Abeta antibody revealed a close connection between restoration of meningeal lymphatic morphology and improved Aβ clearance by the anti-Abeta antibody, with less vascular fibrinogen deposition and myeloid cell activation (which have been linked to worse disease outcome in AD transgenic mice (Ryu, J. K. et al. Fibrin-targeting immunotherapy protects against neuroinflammation and neurodegeneration. Nat Immunol 19, 1212-1223, (2018); Merlini, M. et al. Fibrinogen Induces Microglia-Mediated Spine Elimination and Cognitive Impairment in an Alzheimer's Disease Model. Neuron 101, 1099-1108 e1096, (2019); Hong, S. et al. Complement and microglia mediate early synapse loss in Alzheimer mouse models. Science 352, 712-716, (2016))).

Moreover, treatment of aged J20 mice (14-16 months-old, FIGS. 12G-12K) and of APPswe mice (26-30 months-old, FIGS. 12L-12P) with mVEGF-C improved anti-Abeta antibody-mediated brain Aβ clearance when compared to that in mice treated with the control eGFP-expressing virus.

### Example 9: Discussion

It has been previously shown that induction of meningeal lymphatic dysfunction in young-adult 5×FAD mice results in worsened meningeal amyloid angiopathy (Da Mesquita, S. et al. Functional aspects of meningeal lymphatics in ageing and Alzheimer's disease. Nature 560, 185-191, (2018)). The efficacy of passive immunotherapy with anti-Abeta antibody is greatly reduced in 5×FAD mice with defective meningeal lymphatic drainage, underscoring the importance of early diagnosis and therapeutic intervention in AD patients, preferentially at a stage when the meningeal lymphatic system is still operational. The advanced stage of disease (or simply the advanced age) at which antibody-based therapies are administered might explain their marginal beneficial effects and/or potential deleterious side effects which, as our results suggest, could be attributable, at least in part, to a compromised meningeal lymphatic function. Altogether, results of combination therapy with mVEGF-C, either prophylactic to prevent meningeal lymphatic dysfunction in 4-5 months-old 5×FAD mice, or therapeutic to augment meningeal lymphatic drainage in aged J20 and APPswe mice (both of which develop less brain Aβ pathology and at a later age), show synergistically improved clearance of Aβ by the anti-Abeta antibody. The data support a combination of immunotherapies that target brain Aβ (or potentially other disease-related proteins such as APOE (Liao, F. et al. Targeting of nonlipidated, aggregated apoE with antibodies inhibits amyloid accumulation. J Clin Invest 128, 2144-2155, (2018)), Tau (Yanamandra, K. et al. Anti-tau antibodies that block tau aggregate seeding in vitro markedly decrease pathology and improve cognition in vivo. Neuron 80, 402-414, (2013)) or fibrin (Ryu, J. K. et al. Fibrin-targeting immunotherapy protects against neuroinflammation and neurodegeneration. Nat Immunol 19, 1212-1223, (2018)) with therapeutic strategies aimed at improving meningeal lymphatic function, in order to maximize clearance of these pathological proteinaceous species from the brain in AD. Ultimately, therapeutic targeting of meningeal lymphatic vasculature might be of relevance for other neurodegenerative disorders characterized by protein misfolding and accumulation, such as Huntington's or Parkinson's diseases, where administration of antisense oligonucleotides (Kordasiewicz, H. B. et al. Sustained therapeutic reversal of Huntington's disease by transient repression of huntingtin synthesis. Neuron 74, 1031-1044, (2012)) or of monoclonal antibodies against α-synuclein (Weihofen, A. et al. Development of an aggregate-selective, human-derived alpha-synuclein antibody BIIB054 that ameliorates disease phenotypes in Parkinson's disease models. Neurobiol Dis 124, 276-288, (2019)) into the CSF are also being considered as promising therapeutic strategies.

### Example 10: Materials and Methods

The following materials and methods were used in the studies described in Examples 16-18.

Mouse strains and housing. Adult (2-3 months-old) male C57BL/6J wild type (WT) mice were purchased from the Jackson Laboratory (JAX stock #000664, Bar Harbor, Maine, USA). Aged (20-24 months-old) WT mice were provided by the National Institutes of Health/National Institute on Aging (Bethesda, MD, USA). All mice were maintained in the animal facility for habituation for at least 1 week prior to the start of the experiment. Male hemizygous B6.Cg-Tg(APPSwFlLon,PSEN1*M146L*L286V)6799Vas/Mmjax (5×FAD, JAX stock #008730), B6.Cg-Tg(PDGFB-APPSwInd)20Lms/2Mmjax (J20, JAX stock #006293) and B6.Cg-Tg(APP695)3Dbo (APPswe, JAX stock #005866) were purchased from the Jackson Laboratory and bred in-house on a C57BL/6J background. In-house bred male or female transgene carriers and non-carrier (WT) littermates were used at different ages. The genotype and age of mice from different strains are indicated in figure schemes or legends throughout the manuscript. Male mice were used in the different experiments, unless stated otherwise. Mice of all strains were housed in an environment with controlled temperature and humidity and on a 12-hour light/dark cycle (lights on at 7:00). All mice were fed with regular rodent's chow and sterilized tap water ad libitum. All experiments were approved by the Institutional Animal Care and Use Committee of the University of Virginia.

Intra-cisterna magna and intravenous injections. Mice were anaesthetized by intraperitoneal (i.p.) injection of a mixed solution of ketamine (100 mg/Kg) and xylazine (10 mg/Kg) in saline. The skin of the neck was shaved and cleaned with iodine and 70% ethanol, ophthalmic solution placed on the eyes to prevent drying and the mouse's head was secured in a stereotaxic frame. After making a small (4-5 mm) skin incision, the muscle layers were retracted and the atlantooccipital membrane of the cisterna magna was exposed. Using a Hamilton syringe (coupled to a 33-gauge needle), the volume of the desired solution was injected into the CSF-filled cisterna magna compartment at a rate of ˜2.5 μL/min. After injecting, the syringe was left in place for at least 2 min to prevent back-flow of CSF. The neck skin was then sutured, the mice were allowed to recover in supine position on a heating pad until fully awake and subcutaneously injected with ketoprofen (2 mg/Kg). This method of intra-cisterna magna (i.c.m.) injection was used to administer 5 μL of either Visudyne® (verteporfin for injection, Valeant Ophtalmics), adeno associated viral vectors (AAV1-CMV-mVEGF-C-WPRE and AAV1-CMV-eGFP at 10¹² genome copies per mL, purchased from Vector BioLabs, Philadelphia) or different antibody solutions of murine anti-Abeta antibody or IgG2a (manufactured by Absolute Antibody Ltd., Oxford Centre for Innovation, United Kingdom). Alternatively, antibodies were also injected into the tail vein of mice (i.v.). Antibody dosages/titers are specified in the main text and in each figure legend.

Meningeal lymphatic vessel ablation. Selective ablation of the meningeal lymphatic vessels was achieved by injection of Visudyne (Vis.) and consecutive transcranial photoconversion (photo.) steps following previously described methodology and regimens (Da Mesquita, S. et al. Functional aspects of meningeal lymphatics in ageing and Alzheimer's disease. Nature 560, 185-191, (2018); Louveau, A. et al. CNS lymphatic drainage and neuroinflammation are regulated by meningeal lymphatic vasculature. Nat Neurosci 21, 1380-1391, (2018)). Visudyne was reconstituted following manufacturer instructions, aliquoted and kept at −20° C. until further used. Immediately upon being thawed, Visudyne was injected into the CSF (i.c.m.) and, 15 min later, an incision was performed in the skin to expose the skull bone and photoconvert the drug by pointing a 689-nm wavelength non-thermal red light (Coherent Opal Photoactivator, Lumenis) on 5 different spots above the intact skull (close to the injection site, above the superior sagittal sinus close to the rostral rhinal vein, above the confluence of sinuses and above each transverse sinus). Each spot was irradiated with a light dose of 50 J/cm² at an intensity of 600 mW/cm² for a total of 83 s. Controls were injected with the same volume of Visudyne only, without the photoconversion step. The scalp skin was then sutured, the mice were allowed to recover on a heating pad until fully awake and subcutaneously injected with ketoprofen (2 mg/Kg).

In vivo measurement of CSF outflow into dCLNs. Upon i.c.m. injection of 5 μL of a suspension of 0.5 μm yellow-green fluorescent (505/515 nm) microspheres (FluoSpheres™ carboxylate-modified microspheres, Thermo Fisher Scientific) diluted in artificial CSF (1:1 v/v,) following the procedure described previously, the syringe was left in place for 10 min to prevent backflow and then the mouse was prepared for live imaging of microsphere drainage from the CSF into the dCLNs using a stereomicroscope (M205 FA, Leica Microsystems). The mouse was positioned supine with the head held in position with a length of suture behind the upper incisors and the upper limbs held in place with medical tape. Incisions were made from the center of the clavicle, anterior to the top of the salivary gland and lateral approximately 1 cm. The further preparation was performed on the right side, however in some instances moved to the left side when anatomical variation prevented imaging. The salivary gland was carefully separated at its lateral extent and gently retracted medially. The omohyoid and sternomastoid muscles were retracted laterally, exposing the dCLN. Imaging began approximately 15 minutes after i.c.m. injection. Images were acquired at 25-30 frames per second for a total of 60 seconds. After imaging, mice were euthanized by injection of Euthasol (10% v/v in saline). Fluorescent microsphere drainage was analyzed in FIJI software by drawing a line demarcating the draining lymphatic vessel afferent to the dCLN and manually counting the beads passing the line by a blinded experimenter. Mice were discarded from the analysis due to prior complications during the surgical procedure (e.g. hemorrhages) or due to failure in detecting microspheres draining into the dCLN during image acquisition. In representative images, microspheres were tracked using TrackMate (Tinevez, J. Y. et al. TrackMate: An open and extensible platform for single-particle tracking. Methods 115, 80-90, (2017)) to show the comulative tracks over a 20 see interval.

Tissue collection and processing. Mice were given a lethal dose of anesthetics by intraperitoneal (i.p.) injection of Euthasol (10% v/v in saline) and transcardially perfused with ice cold phosphate buffer saline (PBS, pH 7.4) with heparin (10 U/mL). After stripping the skin and muscle from the bone, the entire head was collected and drop fixed in 4% paraformaldehyde (PFA) for 24 hours at 4° C. After removal of the mandibles and nasal bone, the top of the skull (skull cap) was removed with fine surgical curved scissors (Fine Science Tools) by cutting clockwise, beginning and ending inferior to the right post-tympanic hook and kept in PBS 0.02% azide at 4° C. until further use. Fixed meninges (dura mater and arachnoid) were carefully dissected from the skullcaps with Dumont #5 and #7 fine forceps (Fine Science Tools) and kept in PBS 0.02% azide at 4° C. until further use. Alternatively, the skull was cut sagitally, along the median plane, and after removing the brain, the skull pieces with the attached meningeal layers were kept in PBS 0.02% azide at 4° C. until further use. The brains were kept in 4% PFA for additional 24 hours (48 hours in total). Fixed brains were washed with PBS, cryoprotected with 30% sucrose and frozen in Tissue-Plus® O.C.T. compound (Thermo Fisher Scientific). Fixed and frozen brains were sliced (50 μm thick sections) with a cryostat (Leica) and kept in PBS 0.02% azide at 4° C.

Immunohistochemistry, imaging and quantifications. The following steps were generally applied for free floating brain sections and meningeal whole mounts. When appropriate, prior to immunofluorescent staining, brain sections were stained for amyloid deposits with the Amylo-Glo® RTD™ reagent (Biosensis, Fine Bioscience Tools, South Australia), following manufacturer instructions. For immunofluorescence staining, tissue was rinsed in PBS and incubated with PBS 0.5% Triton X-100 (Thermo Fisher Scientific, PBS-T) for 30 min, followed by PBS-T containing 0.5% of normal serum (either goat or chicken) or 0.5% bovine serum albumin (BSA) for 30 min at room temperature (RT). This blocking step was followed by incubation with appropriate dilutions of primary antibodies: rat anti-LYVE-1-eFluor660 or anti-LYVE-1-Alexa Fluor®488 (eBioscience, clone ALY7, 1:200), Armenian hamster anti-CD31 (Millipore Sigma, MAB1398Z, clone 2H8, 1:200), rabbit anti-AQP4 (Millipore Sigma, A5971, 1:200), goat anti-IBA1 (Abcam, ab5076, polyclonal, 1:200), rat anti-CD68 (BioLegend, 137002, clone FA-11, 1:100), rat anti-LAMP-1 (Abcam, ab25245, clone 1D4B, 1:300), rabbit anti-Fibrinogen (Dako, A0080, polyclonal, 1:200), anti-Aβ_{1-37/42} (Cell Signaling, 8243S, clone D54D2, 1:400) in PBS-T containing 0.5% BSA overnight at 4° C. Meningeal whole mounts or brain sections were then washed 3 times for 10 min at RT in PBS-T followed by incubation with the appropriate rat, chicken, goat or donkey eFluor570 or Alexa Fluor® 488, 594, or 647 conjugated anti-rat, -goat, -rabbit, -mouse or -Armenian hamster IgG antibodies (Thermo Fisher Scientific, 1:500) for 1 hour at RT in PBS-T. After an incubation for 10 min with 1:5000 DAPI in PBS, the tissue was washed 3 times for 5 min with PBS, left to dry at RT (10-20 minutes) and mounted with Shandon™ Aqua-Mount (Thermo Fisher Scientific) and glass coverslips. To stain lymphatic vasculature in the intact skull cap meninges, the same skull hemisphere was incubated in PBS-T 0.5% BSA for 2 hours and then with anti-LYVE-1 eFluor 660 (1:100) in PBS-T 0.5% BSA for 48 hours. Skull caps were then washed 3 times for 1 hour with PBS-T and left washing in PBS-T overnight at 4° C. Skull caps were washed once with PBS kept in PBS at 4° C. Preparations were stored at 4° C. for no more than 1 week until images were acquired. A stereomicroscope (M205 FA, Leica Microsystems) was used to image the meningeal lymphatic vessels within the skull caps. A widefield microscope (DMI6000 B with Adaptive Focus Control, Leica Microsystems) was used for images of Aβ deposits in brain sections and a confocal microscope (FV1200 Laser Scanning Confocal Microscope, Olympus) to acquire all the other images. Upon acquisition, images were opened in the FIJI software for quantification. The ROI (region of interest) manager, Simple Neurite Tracer and Cell Counter plugins were used to measure total lymphatic vessel length and branching points in a particular region of the meningeal whole mount. The Threshold and Measure plugins were used to measure the coverage (as % of ROI or as area in μm²) by Aβ in the brain (in delineated hippocampus, cortex/striatum/amygdala, thalamus/hypothalamus, or whole brain section; plotted values resulted from the average of 3 representative sections per sample) and meninges, as well as LAMP-1, Fibrinogen and IBA1 in images of the brain cortex (plotted values resulted from the average of 4 representative images taken from 2 brain sections per sample). The Analyze Particles plugin (Size, 4-infinity μm²; Circularity, 0.05-1) was used to measure the number of Aβ plaques per mm² of brain region/section and average size of the plaques (μm²). The Cell Counter plugin was also used to quantify the number of peri-Aβ plaque IBA1⁺ cells (cell body within 10 μm of plaque). The Threshold and Image Calculator plugins were used to determine the % of colocalization between the signals of anti-Abeta antibody and CD31, anti-Abeta antibody and Aβ (Amylo-Glo RTD), or IBA1 and CD68 in brain images acquired using the confocal microscope. All measurements were performed by a blinded experimenter, Microsoft Excel was used to calculate average values in each experiment and statistical analysis performed using Prism 7.0a (GraphPad Software, Inc.).

Flow cytometry. Mice were injected with Euthasol (i.p.) and were transcardially perfused with ice cold PBS with heparin. The brains were collected into ice-cold RPMI 1640 (Gibco), and the cortices were dissected after removing hippocampus and remnants of choroid plexus and pia matter. Individual meninges were immediately dissected from the mouse's skull cap in ice-cold RPMI 1640. The tissues were digested for 20 min at 37° C. with 1 mg/mL of Collagenase VIII, 1 mg/mL of Collagenase D and 50 U/mL of DNAse I (all from Sigma Aldrich) in RPMI 1640. The same volume of RPMI with 5% FBS (Atlas Biologicals) and 10 mM EDTA (Thermo Fisher Scientific) was added to the digested tissue, which was then filtered through a 70 μm cell strainer (Fisher Scientific). The cell pellets were washed, resuspended in ice-cold fluorescence-activated cell sorting (FACS) buffer (pH 7.4; 0.1 M PBS; 1 mM EDTA and 1% BSA), preincubated for 10 min at 4° C. with Fc-receptor blocking solution (rat anti-mouse CD16/32, clone 93, BioLegend, 1:200 in FACS) and stained for extracellular markers with the following antibodies (all at 1:200 in FACS): anti-TCRγδ-FITC (11-5811-82, eBioscience), anti-CD45-BB515 (564590, BD Bioscience), anti-NK1.1-PE (553165, BD Bioscience), anti-B220-PE (553090, BD Bioscience), anti-CD4-PerCP-Cy5.5 (550954, BD Bioscience), anti-CD64-PerCP-Cy5.5 (139308, BioLegend), anti-CD8a-PE-Cy7 (552877, BD Bioscience), anti-CD11c-PE-Cy7 (558079, BD Bioscience), anti-PD-1-APC (135210, BioLegend), anti-CD45-A700 (560510, BD Bioscience), anti-CD19-A700 (557958, BD Bioscience), anti-MHC-II-eFluor450 (48-5321-82, eBioscience) and anti-TCRβ-BV510 (563221, BD Bioscience). Cell viability was determined by using the Zombie NIR™ or Zombie AQUA™ Viability Kits following the manufacturer's instructions (BioLegend). After an incubation period of 25 min at 4° C., cells were washed with FACS buffer and fluorescence data was collected with a Gallios™ Flow Cytometer (Beckman Coulter, Inc.). Data was analyzed using FlowJo™ 10 software (Tree Star, Inc.). Briefly, singlets were gated using the height, area and the pulse width of the forward and side scatters and then viable leukocytes were selected as CD45⁺Zombie NIR^{neg} or Zombie AQUA^{neg} (CD45⁺ live). Cells were then gated for the appropriate cell type markers. An aliquot from the unstained single cell suspensions was incubated with ViaStain™ AOPI Staining Solution (CS2-0106, Nexcelom Bioscience) to provide accurate counts for each sample using Cellometer Auto 2000 (Nexcelom Bioscience). Data processing was done with Excel and statistical analysis performed using Prism 7.0a (GraphPad Software, Inc.).

In some embodiments, the method, use, or composition comprises various steps or features that are present as single steps or features (as opposed to multiple steps or features). For example, in one embodiment, the method includes a single administration of a flow modulator, or the composition comprises or consists essentially of a flow modulator for single use. The flow modulator may be present in a single dosage unit effective for increasing flow. A composition or use may comprise a single dosage unit of a flow modulator effective for increasing flow as described herein. Multiple features or components are provided in alternate embodiments. In some embodiments, the method, composition, or use comprises one or more means for flow modulation. In some embodiments, the means comprises a flow modulator.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims. For each method of described herein, relevant compositions for use in the method are expressly contemplated, uses of compositions in the method, and, as applicable, methods of making a medicament for use in the method are also expressly contemplated. For example, for methods of increasing flow that comprise a flow modulator, flow modulators for use in the corresponding method are also contemplated, as are uses of a flow modulator in increasing flow according to the method, as are methods of making a medicament comprising the flow modulator for use in increasing flow.

One skilled in the art will appreciate that, for this and other processes and methods disclosed herein, the functions performed in the processes and methods can be implemented in differing order. Furthermore, the outlined steps and operations are only provided as examples, and some of the steps and operations can be optional, combined into fewer steps and operations, or expanded into additional steps and operations without detracting from the essence of the disclosed embodiments.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. For example, “about 5”, shall include the number 5. For example, the term “about” can indicate that the number differs from the reference number by less than 9%, 8%, 7%, 6%, 5%, 4%, 3%, 2%, or 1%. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

1. A method of increasing clearance of a molecule from the central nervous system in a subject in need thereof, the method comprising:
administering an effective amount of a flow modulator to a meningeal space of the subject, wherein the flow modulator increases the fluid flow in the central nervous system of the subject;
and administering an effective amount of a neurological therapeutic agent to the central nervous system of the subject,
thereby increasing the clearance of the molecule from the central nervous system of the subject.

2. A method of reducing an aggregate of a protein or peptide in the central nervous system of a subject in need thereof, the method comprising:
administering a flow modulator to a meningeal space of the subject, wherein the flow modulator increases the fluid flow in the central nervous system of the subject; and
administering an effective amount of a neurological therapeutic agent to the subject,
thereby reducing the aggregate of the protein or peptide in the subject.

3. A method of reducing a microglial inflammatory response in the central nervous system of a subject in need thereof, the method comprising:
administering an effective amount of a flow modulator to a meningeal space of the subject, wherein the flow modulator increases the fluid flow in the central nervous system of the subject; and
administering an effective amount of a neurological therapeutic agent to the subject,
thereby reducing the microglial inflammatory response in the central nervous system of the subject.

4. A method of reducing neurite dystrophy in the central nervous system of a subject in need thereof, the method comprising:
administering an effective amount of a flow modulator to a meningeal space of the subject, wherein the flow modulator increases the fluid flow in the central nervous system of the subject; and
administering an effective amount of a neurological therapeutic agent to the subject,
thereby reducing neurite dystrophy in the central nervous system of the subject.

5. A method of treating a neurological disease in a subject in need thereof, the method comprising:
administering an effective amount of a flow modulator to a meningeal space of the subject, wherein the flow modulator increases the fluid flow in the central nervous system of the subject; and
administering an effective amount of a neurological therapeutic agent for the neurological disease to the subject,
thereby treating the neurological disease in the subject.

6. The method of any one of claims 1-5, wherein the flow modulator comprises a lymphangiogenic growth factor or a polynucleotide comprising a sequence encoding the lymphangiogenic growth factor.

7. The method of claim 6, wherein the lymphangiogenic growth factor comprises a VEGFR3 agonist or a FGF2, or a variant thereof.

8. The method of claim 7, wherein the VEGFR3 agonist comprises a VEGF-c, a VEGF-d, or a variant thereof.

9. The method of claim 8, wherein the VEGFR3 agonist comprises a VEGF-c, or a variant thereof.

10. The method of any one of claims 1-9, wherein the method increases the clearance of a molecule or reduces an aggregate of a protein or peptide, wherein the molecule or the protein or the peptide is selected from the group consisting of Aβ (amyloid beta), alpha synuclein, fibrin, tau, apolipoprotein E (ApoE), TDP43, prion protein, huntingtin, Huntingtin exon 1, ABri peptide, ADan peptide, fragments of immunoglobulin light chains, fragments of immunoglobulin heavy chains, full or N-terminal fragments of serum amyloid A protein (SAA), transthyretin (TTR), β₂-microglobulin, N-terminal fragments of apolipoproteinA-I (ApoAI), C-terminal extended apolipoprotein A-II (ApoAll), N-terminal fragments of apolipoprotein A-IV (ApoAIV), apolipoprotein C-II (ApoCII), apolipoprotein C-III (ApoAIII), fragments of gelsolin, lysozyme, fragments of fibrinogen α-chain, N-terminal truncated cystatin C, islet amyloid polypeptide (IAPP), calcitonin, atrial natriuretic factor (ANF), N-terminal fragments of prolactin (PRL), insulin, medin, lactotransferrin, odontogenic ameloblast-associated protein (ODAM), pulmonary surfactant-associated protein C (SP-C), leukocyte cell-derived chemotaxin-2 (LECT-2), galectin 7 (Gal-7), Corneodesmosin (CDSN), C-terminal fragments of kerato-epthelin (βih-h3), semenogelin-1 (SGI), proteins S100A8/A9, Enfuvirtide, GSK-3β, MARK, CDK5, tyrosine kinase Fyn, protein phosphatase 2A (PP2A), LRRK2, GBA, NF-κB p65.

11. The method of claim 10, wherein the molecule or the protein or the peptide is selected from the group consisting of amyloid beta, fibrin, tau, apolipoprotein E (ApoE), alpha synuclein, TDP43, and huntingtin.

12. The method of any one of claims 1-11, wherein the neurological therapeutic agent comprises an agent selected from the group consisting of a small molecule, a nucleic acid, a peptide, a protein, an antibody or antigen binding fragment thereof, a recombinant virus, a vaccine, a cell, and any combination thereof.

13. The method of claim 12, wherein the neurological therapeutic agent comprises a small molecule agent.

14. The method of claim 13, wherein the small molecule agent comprises any one or more agents in Table 2.

15. The method of claim 13, wherein the small molecule agent is selected from the group consisting of Donepezil, Galantamine, Rivastigmine, Memantine, Lanabecestat, Atabecestat, Verubecestat, Elenbecestat, Semagacestat, Tarenflurbil, and Brexipiprazole.

16. The method of claim 12, wherein the neurological therapeutic agent comprises an antibody, or an antigen binding fragment thereof.

17. The method of claim 16, wherein the method increases the clearance of a molecule or reduces an aggregate of a protein or peptide, and wherein the antibody or the antigen binding fragment thereof binds to the molecule or the protein or the peptide.

18. The method of claim 17, wherein the molecule or the protein or the peptide is selected from the group consisting of amyloid precursor protein, amyloid beta, fibrin, tau, apolipoprotein E (ApoE), alpha-synuclein, TDP43, and huntingtin.

19. The method of claim 18, wherein the protein is amyloid precursor protein or amyloid beta, and the antibody or the antigen binding fragment thereof is selected from the group consisting of: bapineuzumab, gantenerumab, aducanumab, solanezumab, immunoglobulin, BAN2401, semorinemab, zagotenemab, crenezumab, and an antigen binding fragment thereof.

20. The method of claim 18, wherein the protein is tau, and the antibody or the antigen binding fragment thereof is selected from the group consisting of Gosuranemab, Armanezumab, ABBV-8E12 (AbbVie), PHF1, MC1, DA31, 4E6G7, 6B2G12, TOMA, PHF6, PHF13, HJ9.3, HJ9.4, HJ8.5, 43D, 77E9, AT8, MAb86, pS404 mAb IgG2, pS409-tau, PHF1, Ta9, Ta4, Ta1505, DC8E8, and an antigen binding fragment thereof.

21. The method of claim 18, wherein the protein is alpha-synuclein, and the antibody or the antigen binding fragment thereof is selected from the group consisting of BIIB054 (Biogen), PRX002/RG7935 (Roche), prasinezumab (Roche), PD-1601 (AbbVie), 1H7, 5C1, A1-A6, 9E4, 274, NbSyn87*PEST, NAC32, NAC1, AC14, VH14*PEST, syn303, AB1, Human single-chain Fv D10, D5, syn-O1, syn-O2, syn-O4, mAb47, syn-10H, syn-F1, syn-F2, LS4-2G12, and an antigen binding fragment thereof.

22. The method of claim 18, wherein the antibody or the antigen binding fragment thereof is selected from the group consisting of bapineuzumab, gantenerumab, aducanumab, solanezumab, crenezumab, pepinemab, ozanezumab, AT-1501, BIIB054, PRX002, and an antigen binding fragment thereof.

23. The method of any one of claims 1-22, wherein the flow modulator increases the diameter of the meningeal lymphatic vessel by at least about 5%, about 10%, about 15%, or about 20%.

24. The method of any one of claims 1-23, wherein the method reduces the aggregate of the protein or the peptide by at least about 5%.

25. The method of any one of claims 1-24, wherein the method treats a neurological disease, wherein the neurological disease is selected from the group consisting of: Alzheimer's disease, Parkinson's disease, cerebral edema, amyotrophic lateral sclerosis (ALS), Pediatric Autoimmune Neuropsychiatric Disorders Associated with Streptococcal Infections (PANDAS), meningitis, hemorrhagic stroke, Autism spectrum disorder (ASD), brain tumor, epilepsy, Down's syndrome, hereditary cerebral hemorrhage with amyloidosis-Dutch type (HCHWA-D), Familial Danish/British dementia, dementia with Lewy bodies (DLB), Lewy body (LB) variant of AD, multiple system atrophy (MSA), familial encephalopathy with neuroserpin inclusion bodies (FENIB), frontotemporal dementia (FTD), Huntington's disease (HD), Kennedy disease/spinobulbar muscular atrophy (SBMA), dentatorubropallidoluysian atrophy (DRPLA); spinocerebellar ataxia (SCA) type I, SCA2, SCA3 (Machado-Joseph disease), SCA6, SCA7, SCA17, Creutzfeldt-Jakob disease (CJD), familial CJD, Kuru, Gerstmann-Straussler-Scheinker syndrome (GSS), fatal familial insomnia (FFI), corticobasal degeneration (CBD), progressive supranuclear palsy (PSP), cerebral amyloid angiopathy (CAA), multiple sclerosis (MS), AIDS-related dementia complex, brain injury, traumatic brain injury, brain hemorrhage, subarachnoid brain hemorrhage, stroke, and any combination thereof.

26. The method of any one of claims 1-25, wherein the flow modulator is administered to the subject by a route selected from the group consisting of: intrathecal administration, intraventricular administration, intraparenchymal administration, nasal administration, transcranial administration, contact with cerebral spinal fluid (CSF) of the subject, pumping into CSF of the subject, implantation into the skull or brain, contacting a thinned skull or skull portion of the subject with the flow modulator, expression in the subject of a nucleic acid encoding the flow modulator, and any combination thereof.

27. The method of any one of claims 1-26, wherein the neurological therapeutic agent is administered to the subject by a route selected from the group consisting of: intrathecal administration, intraventricular administration, intraparenchymal administration, nasal administration, transcranial administration, contact with cerebral spinal fluid (CSF) of the subject, pumping into CSF of the subject, implantation into the skull or brain, contacting a thinned skull or skull portion of the subject with the neurological therapeutic agent, expression in the subject of a nucleic acid encoding the neurological therapeutic agent, intravenous infusion, and any combination thereof.

28. The method of claim 26 or 27, wherein the flow modulator and the neurologic therapeutic agent are formulated in a same pharmaceutical composition.

29. The method of claim 26 or 27, wherein the flow modulator and the neurologic therapeutic agent are formulated in different pharmaceutical composition.

30. The method of claim 26 or 27, wherein the flow modulator is administered to the subject prior to, concurrently with, or subsequent to the administration of the neurologic therapeutic agent.

31. The method of claim 26 or 27, wherein the flow modulator is administered via a same route as the neurological therapeutic agent.

32. The method of claim 26 or 27, wherein the flow modulator is administered via a different route to the neurological therapeutic agent.

33. A method of increasing clearance of an amyloid beta, or an amyloid precursor protein, from the central nervous system in a subject in need thereof, the method comprising:
administering an effective amount of a VEGF-c, or a variant thereof, or a polynucleotide encoding the VEGF, or the variant thereof, to a meningeal space of the subject, wherein the VEGF-c, or the variant thereof, increases the fluid flow in the central nervous system of the subject;
and administering an effective amount of a neurological therapeutic agent to the central nervous system of the subject,
thereby increasing the clearance of the molecule from the central nervous system of the subject.

34. A method of treating an Alzheimer's disease in a subject in need thereof, the method comprising:
administering an effective amount of a VEGF-c, or a variant thereof, or a polynucleotide encoding the VEGF, or the variant thereof, to a meningeal space of the subject, wherein the VEGF-c, or the variant thereof, increases the fluid flow in the central nervous system of the subject; and
administering an effective amount of a neurological therapeutic agent for the neurological disease to the subject,
thereby treating the Alzheimer's disease.

35. A method of reducing an amyloid beta plaque in the central nervous system of a subject in need thereof, the method comprising:
administering a VEGF-c, or a variant thereof, a polynucleotide encoding the VEGF, or the variant thereof, to a meningeal space of the subject, wherein the VEGF-c, or the variant thereof increases the fluid flow in the central nervous system of the subject; and
administering an effective amount of a neurological therapeutic agent to the subject, thereby reducing the amyloid beta plaque aggregate of the protein or peptide in the central nervous system of the subject.

36. A method of reducing a microglial inflammatory response in the central nervous system of a subject having an amyloid beta plaque in the central nervous system, the method comprising:
administering an effective amount of a flow modulator to a meningeal space of the subject, wherein the flow modulator increases the fluid flow in the central nervous system of the subject; and
administering an effective amount of a neurological therapeutic agent to the subject,
thereby reducing the microglial inflammatory response in the central nervous system of the subject having an amyloid beta plaque in the central nervous system.

37. A method of reducing neurite dystrophy in the central nervous system of a subject having an amyloid beta in the central nervous system, the method comprising:
administering an effective amount of a flow modulator to a meningeal space of the subject, wherein the flow modulator increases the fluid flow in the central nervous system of the subject; and
administering an effective amount of a neurological therapeutic agent to the subject,
thereby reducing neurite dystrophy in the central nervous system of the subject having an amyloid beta plaque in the central nervous system.

38. The method of any one of claims 33-37, wherein the neurological therapeutic agent is an antibody, or an antigen binding fragment thereof, that specifically binds to amyloid precursor or amyloid beta.

39. The method of claim 38, wherein the antibody or the antigen binding fragment thereof is selected from the group consisting of bapineuzumab, gantenerumab, aducanumab, solanezumab, immunoglobulin, BAN2401, semorinemab, zagotenemab, crenezumab, and an antigen binding fragment thereof.

40. A composition or product combination for a treating a neurological disease, comprising:
an effective amount of a flow modulator; and
an effective amount of a neurological therapeutic agent.

41. The composition or product combination of claim 40, wherein the flow modulator comprises a lymphangiogenic growth factor or a polynucleotide comprising a sequence encoding the lymphangiogenic growth factor.

42. The composition or product combination of claim 41, wherein the lymphangiogenic growth factor comprises a VEGFR3 agonist or a FGF2, or a variant thereof.

43. The composition or product combination of claim 42, wherein the VEGFR3 agonist comprises a VEGF-c, a VEGF-d, or a variant thereof.

44. The composition or product combination of claim 42, wherein the VEGFR3 agonist comprises a VEGF-c, or a variant thereof.

45. The composition or product combination of any one of claims 40-44, wherein the neurological therapeutic agent comprises an agent selected from the group consisting of a small molecule, a nucleic acid, a peptide, a protein, an antibody, a recombinant virus, a cell, and any combination thereof.

46. The composition or product combination of claim 45, wherein the neurological therapeutic agent comprises a small molecule agent.

47. The composition or product combination of claim 46, wherein the small molecule agent comprises any one or more agents in Table 2.

48. The composition or product combination of claim 46, wherein the small molecule agent is selected from the group consisting of Donepezil, Galantamine, Rivastigmine, Memantine, Lanabecestat, Atabecestat, Verubecestat, Elenbecestat, Semagacestat, Tarenflurbil, and Brexipiprazole.

49. The composition or product combination of claim 45, wherein the neurological therapeutic agent comprises an antibody or an antigen binding fragment thereof.

50. The composition or product combination of claim 49, wherein the composition or the product combination increases the clearance of a molecule or reduces an aggregate of a protein or peptide, and wherein the antibody or the antigen binding fragment thereof binds to the molecule or the protein or the peptide.

51. The composition or product combination of claim 50, wherein the molecule or the protein or the peptide is selected from the group consisting of amyloid precursor protein, amyloid beta, fibrin, tau, apolipoprotein E (ApoE), alpha-synuclein, TDP43, and huntingtin.

52. The composition or product combination of claim 51, wherein the protein is amyloid precursor protein or amyloid beta, and the antibody or the antigen binding fragment thereof is selected from the group consisting of: bapineuzumab, gantenerumab, aducanumab, solanezumab, immunoglobulin, BAN2401, semorinemab, zagotenemab, crenezumab, and an antigen binding fragment thereof.

53. The composition or product combination of claim 51, wherein the protein is tau, and the antibody or the antigen binding fragment thereof is selected from the group consisting of Gosuranemab, Armanezumab, ABBV-8E12 (AbbVie), PHF1, MC1, DA31, 4E6G7, 6B2G12, TOMA, PHF6, PHF13, HJ9.3, HJ9.4, HJ8.5, 43D, 77E9, AT8, MAb86, pS404 mAb IgG2, pS409-tau, PHF1, Ta9, Ta4, Ta1505, DC8E8, and an antigen binding fragment thereof.

54. The composition or product combination of claim 51, wherein the protein is alpha-synuclein, and the antibody or the antigen binding fragment thereof is selected from the group consisting of BIIB054 (Biogen), PRX002/RG7935 (Roche), prasinezumab (Roche), PD-1601 (AbbVie), 1H7, 5C1, A1-A6, 9E4, 274, NbSyn87*PEST, NAC32, NAC1, AC14, VH14*PEST, syn303, AB1, Human single-chain Fv D10, D5, syn-O1, syn-O2, syn-O4, mAb47, syn-10H, syn-F1, syn-F2, LS4-2G12, and an antigen binding fragment thereof.

55. The composition or product combination of claim 51, wherein the antibody or the antigen binding fragment thereof is selected from the group consisting of bapineuzumab, gantenerumab, aducanumab, solanezumab, crenezumab, pepinemab, ozanezumab, AT-1501, BIIB054, PRX002, and an antigen binding fragment thereof.

56. The composition or product combination of any one of claims 40-55, wherein the flow modulator is in an effective amount to increases the diameter of the meningeal lymphatic vessel by at least about 5%, about 10%, about 15%, or about 20%.

57. The composition or product combination of any one of claims 40-56, wherein the neurological disease is selected from the group consisting of Alzheimer's disease, Parkinson's disease, cerebral edema, amyotrophic lateral sclerosis (ALS), Pediatric Autoimmune Neuropsychiatric Disorders Associated with Streptococcal Infections (PANDAS), meningitis, hemorrhagic stroke, Autism spectrum disorder (ASD), brain tumor, epilepsy, Down's syndrome, hereditary cerebral hemorrhage with amyloidosis-Dutch type (HCHWA-D), Familial Danish/British dementia, dementia with Lewy bodies (DLB), Lewy body (LB) variant of AD, multiple system atrophy (MSA), familial encephalopathy with neuroserpin inclusion bodies (FENIB), frontotemporal dementia (FTD), Huntington's disease (HD), Kennedy disease/spinobulbar muscular atrophy (SBMA), dentatorubropallidoluysian atrophy (DRPLA); spinocerebellar ataxia (SCA) type I, SCA2, SCA3 (Machado-Joseph disease), SCA6, SCA7, SCA17, Creutzfeldt-Jakob disease (CJD), familial CJD, Kuru, Gerstmann-Straussler-Scheinker syndrome (GSS), fatal familial insomnia (FFI), corticobasal degeneration (CBD), progressive supranuclear palsy (PSP), cerebral amyloid angiopathy (CAA), multiple sclerosis (MS), AIDS-related dementia complex, brain injury, traumatic brain injury, brain hemorrhage, subarachnoid brain hemorrhage, stroke, and any combination thereof.

58. The composition or product combination of any one of claims 40-57, wherein the flow modulator is formulated for administration to the subject by a route selected from the group consisting of: intrathecal administration, intraventricular administration, intraparenchymal administration, nasal administration, transcranial administration, contact with cerebral spinal fluid (CSF) of the subject, pumping into CSF of the subject, implantation into the skull or brain, contacting a thinned skull or skull portion of the subject with the flow modulator, expression in the subject of a nucleic acid encoding the flow modulator, and any combination thereof.

59. The composition or product combination of any one of claims 40-58, wherein the neurological therapeutic agent is formulated for administration to the subject by a route selected from the group consisting of: intrathecal administration, intraventricular administration, intraparenchymal administration, nasal administration, transcranial administration, contact with cerebral spinal fluid (CSF) of the subject, pumping into CSF of the subject, implantation into the skull or brain, contacting a thinned skull or skull portion of the subject with the neurological therapeutic agent, expression in the subject of a nucleic acid encoding the neurological therapeutic agent, intravenous infusion, and any combination thereof.

60. The composition or product combination of claim 58 or 59, wherein the flow modulator and the neurologic therapeutic agent are formulated in a same pharmaceutical composition.

61. The composition or product combination of claim 58 or 59, wherein the flow modulator and the neurologic therapeutic agent are formulated in different pharmaceutical compositions.

62. A composition or product combination for a treating an Alzheimer's disease, comprising: an effective amount of a VEGF, or a variant thereof, or a polynucleotide encoding the VEGF, or the variant thereof, and an effective amount of a neurological therapeutic agent.

63. The composition or product combination of claim 62, wherein the neurological therapeutic agent is an antibody or an antigen binding fragment thereof that specifically binds to amyloid precursor protein or amyloid beta.

64. The composition or product combination of claim 63, wherein the antibody or the antigen binding fragment thereof is selected from the group consisting of bapineuzumab, gantenerumab, aducanumab, solanezumab, immunoglobulin, BAN2401, semorinemab, zagotenemab, crenezumab, and an antigen binding fragment thereof.


SEQUENCE LISTING (5 sequences)

SEQ ID NO: 1 - protein, 419 aa, Homo sapiens
MHLLGFFSVACSLLAAALLPGPREAPAAAAAFESGLDLSDAEPDAGEATAYASKDLEEQLRSVSSVDELMTVLYPEYWKMYKCQLRKGGWQHNREQANLNSRTEETIKFAAAHYNTEILKSIDNEWRKTQCMPREVCIDVGKEFGVATNTFFKPPCVSVYRCGGCCNSEGLQCMNTSTSYLSKTLFEITVPLSQGPKPVTISFANHTSCRCMSKLDVYRQVHSIIRRSLPATLPQCQAANKTCPTNYMWNNHICRCLAQEDFMFSSDAGDDSTDGFHDICGPNKELDEETCQCVCRAGLRPASCGPHKELDRNSCQCVCKNKLFPSQCGANREFDENTCQCVCKRTCPRNQPLNPGKCACECTESPQKCLLKGKKFHHQTCSCYRRPCTNRQKACEPGFSYSEEVCRCVPSYWKRPQMS

SEQ ID NO: 2 - protein, 354 aa, Homo sapiens
MYREWVVVNVFMMLYVQLVQGSSNEHGPVKRSSQSTLERSEQQIRAASSLEELLRITHSEDWKLWRCRLRLKSFTSMDSRSASHRSTRFAATFYDIETLKVIDEEWQRTQCSPRETCVEVASELGKSTNTFFKPPCVNVFRCGGCCNEESLICMNTSTSYISKQLFEISVPLTSVPELVPVKVANHTGCKCLPTAPRHPYSIIRRSIQIPEEDRCSHSKKLCPIDMLWDSNKCKCVLQEENPLAGTEDHSHLQEPALCGPHMMFDEDRCECVCKTPCPKDLIQHPKNCSCFECKESLETCCQKHKLFHPDTCSCEDRCPFHTRPCASGKTACAKHCRFPKEKRAAQGPHSRKNP

SEQ ID NO: 3 - protein, 419 aa, Homo sapiens [sequence omitted]

SEQ ID NO: 4 - protein, 288 aa, Homo sapiens
MVGVGGGDVEDVTPRPGGCQISGRGARGCNGIPGAAAWEAALPRRRPRRHPSVNPRSRAAGSPRTRGRRTEERPSGSRLGDRGRGRALPGGRLGGRGRGRAPERVGGRGRGRGTAAPRAAPAARGSRPGPAGTMAAGSITTLPALPEDGGSGAFPPGHFKDPKRLYCKNGGFFLRIHPDGRVDGVREKSDPHIKLQLQAEERGVVSIKGVCANRYLAMKEDGRLLASKCVTDECFFFERLESNNYNTYRSRKYTSWYVALKRTGQYKLGSKTGPGQKAILFLPMSAKS

SEQ ID NO: 5 - protein, 770 aa, Homo sapiens
MLPGLALLLLAAWTARALEVPTDGNAGLLAEPQIAMFCGRLNMHMNVQNGKWDSDPSGTKTCIDTKEGILQYCQEVYPELQITNVVEANQPVTIQNWCKRGRKQCKTHPHFVIPYRCLVGEFVSDALLVPDKCKFLHQERMDVCETHLHWHTVAKETCSEKSTNLHDYGMLLPCGIDKFRGVEFVCCPLAEESDNVDSADAEEDDSDVWWGGADTDYADGSEDKVVEVAEEEEVAEVEEEEADDDEDDEDGDEVEEEAEEPYEEATERTTSIATTTTTTTESVEEVVREVCSEQAETGPCRAMISRWYFDVTEGKCAPFFYGGCGGNRNNFDTEEYCMAVCGSAMSQSLLKTTQEPLARDPVKLPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPRHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHSFGADSVPANTENEVEPVDARPAADRGLTTRPGSGLTNIKTEEISEVKMDAEFRHDSGYEVHHQKLVFFAEDVGSNKGAIIGLMVGGVVIATVIVITLVMLKKKQYTSIHHGVVEVDAAVTPEERHLSKMQQNGYENPTYKFFEQMQN